(19) United States
(12) Patent Application Publication
(10) Pub. No.: US 2012/0219567 A1
(43) Pub. Date: Aug. 30, 2012

(54) Methods Relating to Peripheral Administration of Nogo Receptor Polypeptides

(75) Inventors: Stephen M. Strittmatter, Guilford, CT (US); Daniel H.S. Lee, Sudbury, MA (US)

(73) Assignees: Biogen Idec MA Inc., Cambridge, MA (US); Yale University, New Haven, CT (US)

(21) Appl. No.: 13/401,590

(22) Filed: Feb. 21, 2012

Related U.S. Application Data
(63) Continuation of application No. 12/439,380, filed on Sep. 1, 2009.
(60) Provisional application No. 60/841,223, filed on Aug. 31, 2006.

(51) Int. Cl. A61K 38/17; A61P 27/02; A61P 21/00; A61K 38/22; A61P 9/00; A61P 25/22; A61P 25/00; A61K 39/395; A61P 25/28; A61P 5/14

(52) U.S. Cl. 424/172.1; 514/17.7; 514/17.8; 514/17.5; 514/9.7

(57) ABSTRACT

This invention relates to methods of treating diseases involving accumulation of Aβ plaques, including Alzheimer's Disease by the peripheral administration of soluble Nogo receptor polypeptides. The invention also provides methods of increasing the plasma to brain ratio of Aβ peptide and enhancing Aβ peptide clearance via peripheral administration of soluble Nogo receptor polypeptides. This invention also provides methods of improving memory function or inhibiting memory loss via the peripheral administration of soluble Nogo receptor polypeptides. The invention also provides methods of decreasing the size and number of Aβ plaques in a mammal via peripheral administration of soluble Nogo receptor polypeptides.

### CROSS-REFERENCE TO RELATED APPLICATIONS

This application is a continuation of application Ser. No. 12/439,380, having a 371(c) date of Sep. 1, 2009, which is the National Phase Application of International Application Number PCT/US2007/019158, filed Aug. 31, 2007, which claims benefit of U.S. Provisional Application No. 60/841,223, filed Aug. 31, 2006.

### REFERENCE TO A SEQUENCE LISTING SUBMITTED ELECTRONICALLY VIA EFS-WEB

The content of the electronically submitted sequence listing (Name: SequenceListing.ascii.txt, Size: 57,999 bytes; and Date of Creation: May 9, 2012) is herein incorporated by reference in its entirety.

### FIELD OF THE INVENTION

This invention relates to neurobiology, neurology and pharmacology. More particularly, this invention relates to methods of treating diseases involving Aβ plaque accumulation, including Alzheimer's Disease by the peripheral administration of soluble Nogo receptor polypeptides. The invention also provides methods of increasing the plasma to brain ratio of Aβ peptide and enhancing Aβ peptide clearance via peripheral administration of soluble Nogo receptor polypeptides. This invention also provides methods of improving memory function or inhibiting memory loss via the peripheral administration of soluble Nogo receptor polypeptides. The invention further provides methods of reducing Aβ plaque size and Aβ plaque number via peripheral administration of soluble Nogo receptor polypeptides.

### BACKGROUND OF THE INVENTION

Neurodegeneration in Alzheimer's Disease (AD) is accompanied by amyloid plaques and neurofibrillary tangles. Glenner et al., Science 297:353-356 (2002). The amyloid plaques are composed primarily of a 40-43 aa Amyloid β (Aβ) peptide that derives from proteolytic cleavage of amyloid precursor protein (APP). Li et al., Proc Natl Acad Sci USA 92:12180-12184 (1995); Sinha et al., Nature 402:537-540 (1999); Vassar et al., Science 286:735-741 (1999). Potential therapies include decreasing Aβ production (Lanz et al., J Pharmacol Exp Ther 305:864-871 (2003)) with secretase inhibitors, increasing Aβ degradation (Frautschy et al., Am J Pathol 140:1389-1399 (1992)) with zinc metalloendopeptidases such as insulin-degrading enzyme (IDE) (Qiu et al., J Biol Chem 273:32730-32738 (1998); Bertram et al., Science 290:2302-2303 (2000)) or neprilysin (NEP) (Yasojima et al., Neurosci Lett 297:97-100 (2001); Iwata et al., Science 292:1550-1552 (2001)), and promoting Ab-specific immunity (Younkin S G, Nat Med 7:18-19 (2001); Morgan et al., Nature 408:982-985 (2000); Lee V M. Proc Natl Acad Sci USA 98:8931-8932 (2001)). However, problems with toxicity and clearing the blood-brain barrier (BBB) have hampered efforts to treat AD. Birmingham K. and Frantz S. Nat Med 8:199-200 (2002) and Orgogozo et al., Neurology 61:46-54 (2003).

The Nogo-66 receptor (NgR1) participates in limiting injury-induced axonal growth and experience-dependent plasticity in the adult brain. Fournier et al., Nature 409:341-346 (2001); McGee A. W. and Strittmatter S. M. Trends Neurosci 26:193-198 (2003); McGee et al., Science 309:2222-2226 (2005). See also PCT Publication Nos. WO 2005/016955, WO 03/031462, WO 2004/014311, and WO 01/51520, as well as U.S. Patent Publications US 2002-0077295 and US 2005-0271655 A1, all of which are incorporated herein by reference in their entireties.

In this role, it serves as a receptor for three myelin inhibitor proteins, Nogo, MAG and OMgp, signaling to activate Rho GTPase in axons. Fournier et al., Nature 409:341-346 (2001); Liu et al., Science 297:1190-1193 (2002); Wang et al., Nature 417:941-944 (2002); Fournier et al., J Neurosci 23:1416-1423 (2003); McGee A. W. and Strittmatter S. M. Trends Neurosci 26:193-198 (2003). In addition, brain NgR1 interacts with APP through its Aβ domain. Park et al., J Neurosci 26:1386-1395 (2006). Moreover, increased levels of brain NgR1 result in reduced Aβ load, while loss of endogenous NgR1 elevates Aβ. Parallel changes in Aβ and secreted APPα plus APPβ suggest that at least a portion of the in vivo effects of brain NgR1 on Aβ levels is mediated by blockade of α/β-secretase activity. However, the high affinity of NgR1 for Aβ and the presence of NgR1 in plaques imply that NgR1 might also regulate the clearance of Aβ. Park et al., J Neurosci 26:1386-1395 (2006).

Immunological methods have been successful in decreasing Aβ plaque burden, as reviewed by Schenk. Schenk D. Nat Rev Neurosci 3:824-828 (2002). Both active and passive immunizations have promoted efflux, inhibited influx, or activated microglia-induced Ab degradation. Weiner H. L. and Selkoe D. J. Nature 420:879-884 (2002); Morgan et al., Nature 408:982-985 (2000); Schenk et al., Nature 400:173-177 (1999). Active immunization with Aβ□1-42 plus adjuvant in PD-mAPP reduced Aβ plaque pathology. Schenk et al., Nature 400:173-177 (1999). Bard et al. demonstrated that humoral immunity is sufficient to reduce plaque burden by triggering antibody trafficking across the blood brain barrier. Bard et al., Nat Med 6:916-919 (2000). In contrast, DeMattos et al., demonstrated that an Aβ antibody reduces Alzheimer pathology without antibody passage across the BBB, implicating a peripheral sink mechanism for anti-Aβ reductions in Alzheimer's pathology. DeMattos et al., Proc Natl Acad Sci USA 98:8850-8855 (2001).

In a range of studies, reducing Aβ burden in brain by immunological means has been associated with improved spatial memory performance in Alzheimer model transgenic mice. However, in several reports, behavioral improvements occurred acutely, prior to any change in plaque density, suggesting the antibody association with particular soluble Aβ species is responsible for improved function. Two non-immunoglobulin proteins, RAGE and gelsolin, have been shown to bind Aβ and, when administered peripherally, to decrease brain Aβ load. Deane et al., Nat Med 9:907-913 (2003); Matsuoka et al., J Neurosci 23:29-33 (2003); Arancio et al., Embo J 23:4096-4105 (2004). Whether Aβ reduction by peripheral non-antibody Aβ-binding proteins is associated with improved cognitive and memory function has not been tested.

### SUMMARY OF THE INVENTION

This invention is based on the discovery that the administration of soluble NgR1 polypeptides peripheral to the central nervous system enhanced Aβ clearance from the brain and improved memory function.

In certain embodiments, the invention includes a method for increasing the plasma to brain ratio of Aβ peptide in a mammal, comprising administering a therapeutically effective amount of a soluble Nogo receptor polypeptide, wherein said administration is peripheral to the central nervous system.

In certain embodiments, the invention includes a method for enhancing Aβ clearance from the brain of a mammal, comprising administering a therapeutically effective amount of a soluble Nogo receptor polypeptide, wherein said administration is peripheral to the central nervous system.

In certain embodiments, the invention includes a method for improving memory function or inhibiting memory loss in a mammal comprising administering a therapeutically effective amount of a soluble Nogo receptor polypeptide, wherein said administration is peripheral to the central nervous system.

In certain embodiments, the invention provides a method of reducing the number of Aβ plaques in the brain of a mammal, comprising administering to a mammal in need thereof a therapeutically effective amount of a soluble Nogo receptor polypeptide, wherein said administration is peripheral to the central nervous system.

In certain embodiments, the invention provides a method of reducing the size of Aβ plaques in the brain of a mammal, comprising administering to a mammal in need thereof a therapeutically effective amount of a soluble Nogo receptor polypeptide, wherein said administration is peripheral to the central nervous system.

In certain embodiments, the invention provides a method of treating a disease associated with Aβ plaque accumulation in a mammal comprising administering to a mammal in need thereof a therapeutically effective amount of a soluble Nogo receptor polypeptide, wherein said administration is peripheral to the central nervous system. In some embodiments, the disease is selected from the group consisting of Alzheimer's disease, mild cognitive impairment, mild-to-moderate cognitive impairment, vascular dementia, cerebral amyloid angiopathy, hereditary cerebral hemorrhage, senile dementia, Down's syndrome, inclusion body myositis, age-related macular degeneration, primary amyloidosis, secondary amyloidosis and a condition associated with Alzheimer's disease. In some embodiments, the condition associated with Alzheimer's disease is selected from the group consisting of hypothyroidism, cerebrovascular disease, cardiovascular disease, memory loss, anxiety, a behavioral dysfunction, a neurological condition, and a psychological condition. In some embodiments, the behavioral dysfunction is selected from the group consisting of apathy, aggression, and incontinence. In some embodiments, the neurological condition is selected from the group consisting of Huntington's disease, amyotrophic lateral sclerosis, acquired immunodeficiency, Parkinson's disease, aphasia, apraxia, agnosia, Pick disease, dementia with Lewy bodies, altered muscle tone, seizures, sensory loss, visual field deficits, incoordination, gait disturbance, transient ischemic attack or stroke, transient alertness, attention deficit, frequent falls, syncope, neuroleptic sensitivity, normal pressure hydrocephalus, subdural hematoma, brain tumor, posttraumatic brain injury, and posthypoxic damage. In some embodiments, the psychological condition is selected from the group consisting of depression, delusions, illusions, hallucinations, sexual disorders, weight loss, psychosis, a sleep disturbance, insomnia, behavioral disinhibition, poor insight, suicidal ideation, depressed mood, irritability, anhedonia, social withdrawal, and excessive guilt. In one embodiment, the mammal is a human.

In some embodiments, the soluble Nogo receptor polypeptide is administered subcutaneously, parenteraly, intravenously, intramuscularly, intraperitoneally, transdermally, inhalationaly or buccally. In one embodiment, the soluble Nogo receptor polypeptide is administered subcutaneously.

In some embodiments, the soluble Nogo receptor polypeptide is 90% identical to a reference amino acid sequence is selected from the group consisting of: (i) amino acids 27 to 310 of SEQ ID NO:2; (ii) amino acids 27 to 344 of SEQ ID NO:2; (iii) amino acids 27 to 445 of SEQ ID NO:2; (iv) amino acids 27 to 309 of SEQ ID NO:2; (v) amino acids 1 to 310 of SEQ ID NO:2; (vi) amino acids 1 to 344 of SEQ ID NO:2; (vii) amino acids 1 to 445 of SEQ ID NO:2; (viii) amino acids 1 to 309 of SEQ ID NO:2; (ix) variants or derivatives of any of said reference amino acid sequences, and (x) a combination of one or more of said reference amino acid sequences or variants or derivatives thereof.

In some embodiments, the soluble NgR1 polypeptide is selected from the group consisting of: (i) amino acids 27 to 310 of SEQ ID NO:2; (ii) amino acids 27 to 344 of SEQ ID NO:2; (iii) amino acids 27 to 445 of SEQ ID NO:2; (iv) amino acids 27 to 309 of SEQ ID NO:2; (v) amino acids 1 to 310 of SEQ ID NO:2; (vi) amino acids 1 to 344 of SEQ ID NO:2; (vii) amino acids 1 to 445 of SEQ ID NO:2; (viii) amino acids 1 to 309 of SEQ ID NO:2; (ix) variants or derivatives of any of said polypeptides; and (x) a combination of one or more of said polypeptides or variants or derivatives thereof. In one embodiment, the soluble Nogo receptor polypeptide comprises amino acids 27 to 310 of SEQ ID NO:2. In one embodiment, the soluble Nogo receptor polypeptide comprises amino acids 27 to 344 of SEQ ID NO:2. In one embodiment, the soluble Nogo receptor polypeptide comprises amino acids 27 to 445 of SEQ ID NO:2. In one embodiment, the soluble Nogo receptor polypeptide comprises amino acids 27 to 309 of SEQ ID NO:2. In one embodiment, the soluble Nogo receptor polypeptide comprises amino acids 1 to 310 of SEQ ID NO:2. In one embodiment, the soluble Nogo receptor polypeptide comprises amino acids 1 to 344 of SEQ ID NO:2. In one embodiment, the soluble Nogo receptor polypeptide comprises amino acids 1 to 445 of SEQ ID NO:2. In one embodiment, the soluble Nogo receptor polypeptide comprises amino acids 1 to 309 of SEQ ID NO:2.

In some embodiments, the soluble Nogo receptor polypeptide comprises a first polypeptide fragment and a second polypeptide fragment, wherein said first polypeptide fragment comprises an amino acid sequence identical to a first reference amino acid sequence, except for up to twenty individual amino acid substitutions, wherein said first reference amino acid sequence is selected from the group consisting of: (a) amino acids a to 445 of SEQ ID NO:2, (b) amino acids 27 to b of SEQ ID NO:2, and (c) amino acids a to b of SEQ ID NO:2, wherein a is any integer from 25 to 35, and b is any integer from 300 to 450; wherein said second polypeptide fragment comprises an amino acid sequence identical to a second reference amino acid sequence, except for up to twenty individual amino acid substitutions, wherein said second reference amino acid sequence is selected from the group consisting of (a) amino acids c to 445 of SEQ ID NO:2, (b) amino acids 27 to d of SEQ ID NO:2, and (c) amino acids c to d of SEQ ID NO:2, wherein c is any integer from 25 to 35, and d is any integer from 300 to 450. In some embodiments, the first polypeptide fragment is situated upstream of said second polypeptide fragment. In a further embodiment, a peptide linker is situated between the first polypeptide fragment and the second polypeptide fragment. In one embodiment, the peptide linker comprises SEQ ID NO:18 (G4S)₃.

In some embodiments, at least one amino acid residue of the soluble NgR1 polypeptide is substituted with a different amino acid. In some embodiments, the different amino acid is selected from the group consisting of: alanine, serine and threonine. In one embodiment, the different amino acid is alanine.

In some embodiments, the soluble NgR polypeptides are cyclic. In some embodiments, the cyclic polypeptides further comprise a first molecule linked at the N-terminus and a second molecule linked at the C-terminus; wherein the first molecule and the second molecule are joined to each other to form said cyclic molecule. In some embodiments, the first and second molecules are selected from the group consisting of: a biotin molecule, a cysteine residue, and an acetylated cysteine residue. In some embodiments, the first molecule is a biotin molecule attached to the N-terminus and the second molecule is a cysteine residue attached to the C-terminus of the polypeptide of the invention. In some embodiments, the first molecule is an acetylated cysteine residue attached to the N-terminus and the second molecule is a cysteine residue attached to the C-terminus of the polypeptide of the invention. In some embodiments, the first molecule is an acetylated cysteine residue attached to the N-terminus and the second molecule is a cysteine residue attached to the C-terminus of the polypeptide of the invention. In some embodiments, the C-terminal cysteine has an NH2 moiety attached.

In some embodiments, the soluble NgR1 polypeptide further comprises a non-NgR1 moiety. In some embodiments, the non-NgR1 moiety is a heterologous polypeptide fused to the soluble NgR1 polypeptide. In some embodiments, the invention further provides that the heterologous polypeptide is selected from the group consisting of: (a) serum albumin, (b) an Fc region, (c) a signal peptide, (d) a polypeptide tag, and (e) a combination of two or more of said heterologous polypeptides. In some embodiments, the invention further provides that the Fc region is selected from the group consisting of: an IgA Fc region; an IgD Fc region; an IgG Fc region, an IgEFc region; and an IgM Fc region. In one embodiment, the Fc region is an IgG Fc region. In some embodiments, the invention further provides that a peptide linker is situated between the amino acid sequence and the IgG Fc region. In one embodiment, the peptide linker comprises SEQ ID NO:19(G4S)₂. In some embodiments, the invention further provides that the polypeptide tag is selected from the group consisting of: FLAG tag; Strep tag; poly-histidine tag; VSV-G tag; influenza virus hemagglutinin (HA) tag; and c-Myc tag.

In some embodiments, the invention provides a polypeptide of the invention attached to one or more polyalkylene glycol moieties. In some embodiments, the invention further provides that the one or more polyalkylene glycol moieties is a polyethylene glycol (PEG) moiety. In some embodiments, the invention further provides a polypeptide of the invention attached to 1 to 5 PEG moieties.

In some embodiments, the invention provides that the therapeutically effective amount is from 0.001 mg/kg to 10 mg/kg of soluble Nogo receptor polypeptide. In some embodiments, the therapeutically effective amount is from 0.01 mg/kg to 1 mg/kg of soluble Nogo receptor polypeptide. In some embodiments, the therapeutically effective amount is from 0.05 mg/kg to 0.5 mg/kg of soluble Nogo receptor polypeptide.

In some embodiments, the invention provides that the soluble Nogo receptor polypeptide does not cross the blood-brain barrier.

In some embodiments, the soluble Nogo receptor polypeptide is coadministered with one or more anti-Aβ antibodies. In some embodiments, the soluble Nogo receptor polypeptide is coadministered with one or more additional therapeutic agents, selected from the group consisting of an adrenergic agent, anti-adrenergic agent, anti-androgen agent, anti-anginal agent, anti-anxiety agent, anticonvulsant agent, antidepressant agent, anti-epileptic agent, antihyperlipidemic agent, antihyperlipoproteinemic agent, antihypertensive agent, anti-inflammatory agent, antiobessional agent, antiparkinsonian agent, antipsychotic agent, adrenocortical steroid; adrenocortical suppressant; aldosterone antagonist; amino acid; anabolic steroid; analeptic agent; androgen; blood glucose regulator; cardioprotectant agent; cardiovascular agent; cholinergic agonist or antagonist; cholinesterase deactivator or inhibitor, cognition adjuvant or enhancer; dopaminergic agent; enzyme inhibitor, estrogen, free oxygen radical scavenger; GABA agonist; glutamate antagonist; hormone; hypocholesterolemic agent; hypolipidemic agent; hypotensive agent; immunizing agent; immunostimulant agent; monoamine oxidase inhibitor, neuroprotective agent; NMDA antagonist; AMPA antagonist, competitive or-non-competitive NMDA antagonist; opioid antagonist; potassium channel opener; non-hormonal sterol derivative; post-stroke and post-head trauma treatment; prostaglandin agent; psychotropic agent; relaxant agent; sedative agent; sedative-hypnotic agent; selective adenosine antagonist; serotonin antagonist; serotonin inhibitor; selective serotonin uptake inhibitor; serotonin receptor antagonist; sodium and calcium channel blocker; steroid; stimulant; and thyroid hormone and inhibitor agents.

### BRIEF DESCRIPTION OF THE DRAWINGS

FIG. 1A shows deletion mapping of the AP-Aβ(1-28) region required for binding to COS-7 cells expressing wild type NgR1. FIG. 1B shows that AP-Aβ (1-28) does not bind to COS-7 cells expressing p75-NTR or RAGE under conditions that allow binding to cells expressing NgR1. FIG. 1C shows displacement of Aβ-Aβ□(1-28) but not AP-Nogo-66(1-33), from NgR1 by Aβ□(1-28).

FIG. 2A shows mutant NgR1 proteins at the surface of transfected COS-7 cells were detected by immunostaining with rabbit anti-NgR1 antibody recognized by anti-rabbit-AP. FIG. 2B shows binding of AP or AP fused NgR1 ligands to COS-7 cells expressing NgR1 mutants displaying differential binding. FIG. 2C depicts cell lysate of COS-7 cells expressing NgR1 and mutants that were immunoblotted with anti-NgR1 antibody to ascertain molecular weight and expression levels. FIG. 2D shows quantification of AP binding of NgR1 ligands to NgR1 mutants expressed as a percentage of wild type NgR1.

FIG. 3A shows an anti-NgR1 immunoblot of protein concentrated by protein A/G affinity chromotography in brain lysate of APPswe/PSEN-1ΔE9 transgenic mice treated subcutaneously with rat IgG, subcutaneously with NgR1(310)ecto-Fc or i.c.v. with NgR1(310)ecto-Fc. FIG. 3B shows ratio of plasma versus brain Aβ level in peripherally-treated Appswe/PSEN-1ΔE9 transgenic mice at 10 months of age plotted as a percentage.

FIG. 3C shows an anti-APP (6E10) Immunoblot of brain lysate of APPswe/PSEN-1ΔE9 transgenic mice treated subcutaneously with rat IgG or subcutaneously with NgR1(310)ecto-Fc from 7-10 months of age. FIG. 3D shows the level of anti-APP immunoreactivity in brain lysates.

FIG. 4A shows examples of anti-Aβ immunoreactive plaque deposits in cerebral cortex of control and NgR1(310)ecto-Fc treated transgenic mice. FIG. 4B shows examples of anti-synaptophysin immunoreactive plaque deposits in hippocampus. FIG. 4C shows anti-GFAP immunoreactivity in the hippocampus. FIG. 4D shows the percentage of area occupied by Aβ plaque quantified from images in FIG. 4A. FIG. 4E shows Aβ(1-40) and Aβ(1-42) levels assessed by ELISA between NgR1(310)ecto-Fc and rat IgG groups. FIG. 4F shows the area occupied by anti-synaptophysin immunoreactive dystrophic neurites from FIG. 4B. FIG. 4G shows the percentage of area occupied by anti-GFAP immunoreactivity as measured from images in FIG. 4C.

FIG. 5A shows the average number of errors in a six-arm radial water maze for APPswe/PSEN-1ΔE9 and wild type litter mates at 4 months of age. FIG. 5B shows the average number of errors in a six-arm radial water maze for APPswe/PSEN-10E9 and wild type litter mates at 13 months of age. FIG. 5C shows the average number of errors in a six-arm radial water maze for NgR +/− and NgR−/− mice. FIG. 5D shows the results from subcutaneous treatment of NgR1(310)ecto-Fc in APPswe/PSEN-1ΔE9 mice from months 7-10. FIG. 5E shows a scatter plot between plaque density and average errors per swim for the last ten trials for each mouse.

FIG. 6 shows the visible platform escape latency of APPswe/PSEN-10E9 transgenic mice after subcutaneous treatment with NgR1(310)ecto-Fc or IgG from age 7 months to 10 months.

### DETAILED DESCRIPTION OF THE INVENTION

### Definitions and General Techniques

Unless defined otherwise, all technical and scientific terms used herein have the same meaning as commonly understood by one of ordinary skill in the art to which this invention belongs. In case of conflict, the present application including the definitions will control. Also, unless otherwise required by context, singular terms shall include pluralities and plural terms shall include the singular. All publications, patents and other references mentioned herein are incorporated by reference in their entireties for all purposes.

Although methods and materials similar or equivalent to those described herein can be used in practice or testing of the present invention, suitable methods and materials are described below. The materials, methods and examples are illustrative only, and are not intended to be limiting. Other features and advantages of the invention will be apparent from the detailed description and from the claims.

Throughout this specification and claims, the word “comprise,” or variations such as “comprises” or “comprising,” will be understood to imply the inclusion of a stated integer or group of integers but not the exclusion of any other integer or group of integers.

In order to further define this invention, the following terms and definitions are herein provided.

It is to be noted that the term “a” or “an” entity, refers to one or more of that entity; for example, “an immunoglobulin molecule,” is understood to represent one or more immunoglobulin molecules. As such, the terms “a” (or “an”), “one or more,” and “at least one” can be used interchangeably herein.

As used herein, the term “consists of,” or variations such as “consist of” or “consisting of,” as used throughout the specification and claims, indicate the inclusion of any recited integer or group of integers, but that no additional integer or group of integers may be added to the specified method, structure or composition.

As used herein, the term “consists essentially of,” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure or composition.

As used herein, “antibody” means an intact immunoglobulin, or an antigen-binding fragment thereof. Antibodies of this invention can be of any isotype or class (e.g., M, D, G, E and A) or any subclass (e.g., G1-4, A 1-2) and can have either a kappa (κ) or lambda (λ) light chain.

As used herein, “Fc” means a portion of an immunoglobulin heavy chain that comprises one or more heavy chain constant region domains, CH1, CH2 and CH3: For example, a portion of the heavy chain constant region of an antibody that is obtainable by papain digestion.

As used herein and in U.S. patent application 60/402,866, “Nogo receptor,” “NogoR,” “NogoR-1,” “NgR,” “NgR-1,” “NgR1” and “NOR1” each means Nogo receptor-1.

As used herein, “NogoR fusion protein” means a protein comprising a soluble Nogo receptor-1 moiety fused to a heterologous polypeptide.

As used herein, “humanized antibody” means an antibody in which at least a portion of the non-human sequences are replaced with human sequences. Examples of how to make humanized antibodies may be found in U.S. Pat. Nos. 6,054,297, 5,886,152 and 5,877,293.

As used herein, “chimeric antibody” means an antibody that contains one or more regions from a first antibody and one or more regions from at least one other antibody. The first antibody and the additional antibodies can be from the same or different species.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis.

A polypeptide of the invention may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides may have a defined three-dimensional structure, although they do not necessarily have such structure. Polypeptides with a defined three-dimensional structure are referred to as folded, and polypeptides which do not possess a defined three-dimensional structure, but rather can adopt a large number of different conformations, and are referred to as unfolded. As used herein, the term glycoprotein refers to a protein coupled to at least one carbohydrate moiety that is attached to the protein via an oxygen-containing or a nitrogen-containing side chain of an amino acid residue, e.g., a serine residue or an asparagine residue.

By an “isolated” polypeptide or a fragment, variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated for purposed of the invention, as are native or recombinant polypeptides which have been separated, fractionated, or partially or substantially purified by any suitable technique.

In the present invention, a “polypeptide fragment” refers to a short amino acid sequence of a larger polypeptide. Protein fragments may be “free-standing,” or comprised within a larger polypeptide of which the fragment forms a part of region. Representative examples of polypeptide fragments of the invention, include, for example, fragments comprising about 5 amino acids, about 10 amino acids, about 15 amino acids, about 20 amino acids, about 30 amino acids, about 40 amino acids, about 50 amino acids, about 60 amino acids, about 70 amino acids, about 80 amino acids, about 90 amino acids, and about 100 amino acids or more in length.

The terms “fragment,” “variant,” “derivative” and “analog” when referring to a polypeptide of the present invention include any polypeptide which retains at least some biological activity. Polypeptides as described herein may include fragment, variant, or derivative molecules therein without limitation, so long as the polypeptide still serves its function. NgR1 polypeptides and polypeptide fragments of the present invention may include proteolytic fragments, deletion fragments and in particular, fragments which more easily reach the site of action when delivered to an animal. Polypeptide fragments further include any portion of the polypeptide which comprises an antigenic or immunogenic epitope of the native polypeptide, including linear as well as three-dimensional epitopes. NgR1 polypeptides and polypeptide fragments of the present invention may comprise variant regions, including fragments as described above, and also polypeptides with altered amino acid sequences due to amino acid substitutions, deletions, or insertions. Variants may occur naturally, such as an allelic variant. By an “allelic variant” is intended alternate forms of a gene occupying a given locus on a chromosome of an organism. Genes II, Lewin, B., ed., John Wiley & Sons, New York (1985). Non-naturally occurring variants may be produced using art-known mutagenesis techniques. NgR1 polypeptides and polypeptide fragments of the invention may comprise conservative or non-conservative amino acid substitutions, deletions or additions. NgR1 polypeptides and polypeptide fragments of the present invention may also include derivative molecules. Variant polypeptides may also be referred to herein as “polypeptide analogs.” As used herein a “derivative” of a polypeptide or a polypeptide fragment refers to a subject polypeptide having one or more residues chemically derivatized by reaction of a functional side group. Also included as “derivatives” are those peptides which contain one or more naturally occurring amino acid derivatives of the twenty standard amino acids. For example, 4-hydroxyproline may be substituted for proline; 5-hydroxylysine may be substituted for lysine; 3-methylhistidine may be substituted for histidine; homoserine may be substituted for serine; and ornithine may be substituted for lysine.

As used herein the term “disulfide bond” includes the covalent bond formed between two sulfur atoms. The amino acid cysteine comprises a thiol group that can form a disulfide bond or bridge with a second thiol group.

As used herein, “fusion protein” means a protein comprising a first polypeptide linearly connected, via peptide bonds, to a second, polypeptide. The first polypeptide and the second polypeptide may be identical or different, and they may be directly connected, or connected via a peptide linker (see below).

The term “polynucleotide” is intended to encompass a singular nucleic acid as well as plural nucleic acids, and refers to an isolated nucleic acid molecule or construct, e.g., messenger RNA (mRNA) or plasmid DNA (pDNA). A polynucleotide can contain the nucleotide sequence of the full-length cDNA sequence, including the untranslated 5′ and 3′ sequences, the coding sequences. A polynucleotide may comprise a conventional phosphodiester bond or a non-conventional bond (e.g., an amide bond, such as found in peptide nucleic acids (PNA)). The polynucleotide can be composed of any polyribonucleotide or polydeoxyribonucleotide, which may be unmodified RNA or DNA or modified RNA or DNA. For example, polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, the polynucleotides can be composed of triple-stranded regions comprising RNA or DNA or both RNA and DNA. polynucleotides may also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

The term “nucleic acid” refer to any one or more nucleic acid segments, e.g., DNA or RNA fragments, present in a polynucleotide. By “isolated” nucleic acid or polynucleotide is intended a nucleic acid molecule, DNA or RNA, which has been removed from its native environment. For example, a recombinant polynucleotide encoding an NgR1 polypeptide or polypeptide fragment of the invention contained in a vector is considered isolated for the purposes of the present invention. Further examples of an isolated polynucleotide include recombinant polynucleotides maintained in heterologous host cells or purified (partially or substantially) polynucleotides in solution. Isolated RNA molecules include in vivo or in vitro RNA transcripts of polynucleotides of the present invention. Isolated polynucleotides or nucleic acids according to the present invention further include such molecules produced synthetically. In addition, polynucleotide or a nucleic acid may be or may include a regulatory element such as a promoter, ribosome binding site, or a transcription terminator.

As used herein, a “coding region” is a portion of nucleic acid which consists of codons translated into amino acids. Although a “stop codon” (TAG, TGA, or TAA) is not translated into an amino acid, it may be considered to be part of a coding region, but any flanking sequences, for example promoters, ribosome binding sites, transcriptional terminators, introns, and the like, are not part of a coding region. Two or more coding regions of the present invention can be present in a single polynucleotide construct, e.g., on a single vector, or in separate polynucleotide constructs, e.g., on separate (different) vectors. Furthermore, any vector may contain a single coding region, or may comprise two or more coding regions, e.g., a single vector may separately encode an immunoglobulin heavy chain variable region and an immunoglobulin light chain variable region. In addition, a vector, polynucleotide, or nucleic acid of the invention may encode heterologous coding regions, either fused or unfused to a nucleic acid encoding an NgR1 polypeptide or polypeptide fragment of the present invention. Heterologous coding regions include without limitation specialized elements or motifs, such as a secretory signal peptide or a heterologous functional domain.

In certain embodiments, the polynucleotide or nucleic acid is DNA. In the case of DNA, a polynucleotide comprising a nucleic acid which encodes a polypeptide normally may include a promoter and/or other transcription or translation control elements operably associated with one or more coding regions. An operable association is when a coding region for a gene product, e.g., a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter may be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription. Suitable promoters and other transcription control regions are disclosed herein.

A variety of transcription control regions are known to those skilled in the art. These include, without limitation, transcription control regions which function in vertebrate cells, such as, but not limited to, promoter and enhancer segments from cytomegaloviruses (the immediate early promoter, in conjunction with intron-A), simian virus 40 (the early promoter), and retroviruses (such as Rous sarcoma virus). Other transcription control regions include those derived from vertebrate genes such as actin, heat shock protein, bovine growth hormone and rabbit β-globin, as well as other sequences capable of controlling gene expression in eukaryotic cells. Additional suitable transcription control regions include tissue-specific promoters and enhancers as well as lymphokine-inducible promoters (e.g., promoters inducible by interferons or interleukins).

Similarly, a variety of translation control elements are known to those of ordinary skill in the art. These include, but are not limited to ribosome binding sites, translation initiation and termination codons, and elements derived from picornaviruses (particularly an internal ribosome entry site, or IRES, also referred to as a CITE sequence).

In other embodiments, a polynucleotide of the present invention is RNA, for example, in the form of messenger RNA (mRNA).

Polynucleotide and nucleic acid coding regions of the present invention may be associated with additional coding regions which encode secretory or signal peptides, which direct the secretion of a polypeptide encoded by a polynucleotide of the present invention. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells generally have a signal peptide fused to the N-terminus of the polypeptide, which is cleaved from the complete or “full length” polypeptide to produce a secreted or “mature” form of the polypeptide. In certain embodiments, the native signal peptide, e.g., an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, may be used. For example, the wild-type leader sequence may be substituted with the leader sequence of human tissue plasminogen activator (TPA) or mouse β-glucuronidase.

As used herein the term “engineered” includes manipulation of nucleic acid or polypeptide molecules by synthetic means (e.g. by recombinant techniques, in vitro peptide synthesis, by enzymatic or chemical coupling of peptides or some combination of these techniques).

As used herein, the terms “linked,” “fused” and “fusion” are used interchangeably. These terms refer to the joining together of two more elements or components, by whatever means including chemical conjugation or recombinant means. An “in-frame fusion” refers to the joining of two or more polynucleotide open reading frames (ORFs) to form a continuous longer ORF, in a manner that maintains the correct translational reading frame of the original ORFs. Thus, a recombinant fusion protein is a single protein containing two ore more segments that correspond to polypeptides encoded by the original ORFs (which segments are not normally so joined in nature.) Although the reading frame is thus made continuous throughout the fused segments, the segments may be physically or spatially separated by, for example, in-frame linker sequence.

A “linker” sequence is a series of one or more amino acids separating two polypeptide coding regions in a fusion protein. A typical linker comprises at least 5 amino acids. Additional linkers comprise at least 10 or at least 15 amino acids. In certain embodiments, the amino acids of a peptide linker are selected so that the linker is hydrophilic. The linker (Gly-Gly-Gly-Gly-Ser)₃ (G4S)₃ (SEQ ID NO:18) is a preferred linker that is widely applicable to many antibodies as it provides sufficient flexibility. Other linkers include (Gly-Gly-Gly-Gly-Ser)₂ (G4S)₂ (SEQ ID NO:19), Glu Ser Gly Arg Ser Gly Gly Gly Gly Ser Gly Gly Gly Gly Ser (SEQ ID NO:20), Glu Gly Lys Ser Ser Gly Ser Gly Ser Glu Ser Lys Ser Thr (SEQ ID NO:21), Glu Gly Lys Ser Ser Gly Ser Gly Ser Glu Ser Lys Ser Thr Gln (SEQ ID NO:22), Glu Gly Lys Ser Ser Gly Ser Gly Ser Glu Ser Lys Val Asp (SEQ ID NO:23), Gly Ser Thr Ser Gly Ser Gly Lys Ser Ser Glu Gly Lys Gly (SEQ ID NO:24), Lys Glu Ser Gly Ser Val Ser Ser Glu Gln Leu Ala Gln Phe Arg Ser Leu Asp (SEQ ID NO:25), and Glu Ser Gly Ser Val Ser Ser Glu Glu Leu Ala Phe Arg Ser Leu Asp (SEQ TD NO:26). Examples of shorter linkers include fragments of the above linkers, and examples of longer linkers include combinations of the linkers above, combinations of fragments of the linkers above, and combinations of the linkers above with fragments of the linkers above.

In the context of polypeptides, a “linear sequence” or a “sequence” is an order of amino acids in a polypeptide in an amino to carboxyl terminal direction in which residues that neighbor each other in the sequence are contiguous in the primary structure of the polypeptide.

The term “expression” as used herein refers to a process by which a gene produces a biochemical, for example, an RNA or polypeptide. The process includes any manifestation of the functional presence of the gene within the cell including, without limitation, gene knockdown as well as both transient expression and stable expression. It includes, without limitation, transcription of the gene into messenger RNA (mRNA), transfer RNA (tRNA), small hairpin RNA (shRNA), small interfering RNA (siRNA) or any other RNA product, and the translation of such mRNA into polypeptide(s), as well as any processes which regulate either transcription or translation. If the final desired product is a biochemical, expression includes the creation of that biochemical and any precursors. Expression of a gene produces a “gene product.” As used herein, a gene product can be either a nucleic acid, e.g., a messenger RNA produced by transcription of a gene, or a polypeptide which is translated from a transcript. Gene products described herein further include nucleic acids with post transcriptional modifications, e.g., polyadenylation, or polypeptides with post translational modifications, e.g., methylation, glycosylation, the addition of lipids association with other protein subunits, proteolytic cleavage, and the like.

As used herein, the terms “treat” or “treatment” refer to both therapeutic treatment and prophylactic or preventative measures, wherein the object is to prevent or slow down (lessen) an undesired physiological change or disorder, such as the progression of multiple sclerosis. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

By “subject” or “individual” or “animal” or “patient” or “mammal,” is meant any subject, particularly a mammalian subject, for whom diagnosis, prognosis, or therapy is desired. Mammalian subjects include, but are not limited to, humans, domestic animals, farm animals, zoo animals, sport animals, pet animals such as dogs, cats, guinea pigs, rabbits, rats, mice, horses, cattle, cows; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; rodents such as mice, rats, hamsters and guinea pigs; and so on. In certain embodiments, the mammal is a human subject.

As used herein, a “therapeutically effective amount” refers to an amount effective, at dosages and for periods of time necessary, to achieve the desired therapeutic result. A therapeutic result may be, e.g., lessening of symptoms, prolonged survival, improved mobility, and the like. A therapeutic result need not be a “cure”.

As used herein, a “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

As used herein, “Aβ clearance” refers to a shift of Aβ peptide from the brain to the plasma.

Nogo Receptor-1 Polypeptides

The human NgR1 polypeptide is shown below as SEQ ID NO:2.

Full-Length Human NgR1 (SEQ ID NO: 2):
MKRASAGGSRLLAWVLWLQAWQVAAPCPGACVCYNEPKVTTSCPQQGLQ
AVPVGTPAASQRIFLHGNRISHVPAASFRACRNLTILWLHSNVLARIDA
AAFTGLALLEQLDLSDNAQLRSVDPATFHGLGRLHTLHLDRCGLQELGP
GLFRGLAALQYLYLQDNALQALPDDTFRDLGNLTHLFLHGNRISSVPER
AFRGLHSLDRLLLHQNRVAHVHPHAFRDLGRLMTLYLFANNLSALPTEA
LAPLRALQYLRLNDNPWVCDCRARPLWAWLQKFRGSSSEVPCSLPQRLA
GRDLKRLAANDLQGCAVATGPYHPIWTGRATDEEPLGLPKCCQPDAADK
ASVLEPGRPASAGNALKGRVPPGDSPPGNGSGPRHINDSPFGTLPGSAE
PPLTAVRPEGSEPPGFPTSGPRRRPGCSRKNRTRSHCRLGQAGSGGGGT
GDSEGSGALPSLTCSLTPLGLALVLWTVLGPC

The rat NgR1 polypeptide is shown below as SEQ ID NO:4.

Full-Length Rat NgR1 (SEQ ID NO: 4):
MKRASSGGSRLLAWVLWLQAWRVATPCPGACVCYNEPKVTTSCPQQGLQ
AVPTGIPASSQRIFLHGNRISHVPAASFQSCRNLTILWLHSNALARIDA
AAFTGLTLLEQLDLSDNAQLHVVDPTTFHGLGHLHTLHLDRCGLRELGP
GLFRGLAALQYLYLQDNNLQALPDNTFRDLGNLTHLFLHGNRIPSVPEH
AFRGLHSLDRLLLHQNHVARVHPHAFRDLGRLMTLYLFANNLSMLPAEV
LMPLRSLQYLRLNDNPWVCDCRARPLWAWLQKFRGSSSEVPCNLPQRLA
DRDLKRLAASDLEGCAVASGPFRPIQTSQLTDEELLSLPKCCQPDAADK
ASVLEPGRPASAGNALKGRVPPGDTPPGNGSGPRHINDSPFGTLPSSAE
PPLTALRPGGSEPPGLPTTGPRRRPGCSRKNRTRSHCRLGQAGSGASGT
GDAEGSGALPALACSLAPLGLALVLWTVLGPC

The mouse NgR1 polypeptide is shown below as SEQ ID NO:6.

Full-Length Mouse NgR1 (SEQ ID NO: 6):
MKRASSGGSRLLAWVLWLQAWRVATPCPGACVCYNEPKVTTSCPQQGLQ
AVPTGIPASSQRIFLHGNRISHVPAASFQSCRNLTILWLHSNALARIDA
AAFTGLTLLEQLDLSDNAQLHVVDPTTFHGLGHLHTLHLDRCGLRELGP
GLFRGLAALQYLYLQDNNLQALPDNTFRDLGNLTHLFLHGNRIPSVPEH
AFRGLHSLDRLLLHQNHVARVHPHAFRDLGRLMTLYLFANNLSMLPAEV
LMPLRSLQYLRLNDNPWVCDCRARPLWAWLQKFRGSSSEVPCNLPQRLA
DRDLKRLAASDLEGCAVASGPFRPIQTSQLTDEELLSLPKCCQPDAADK
ASVLEPGRPASAGNALKGRVPPGDTPPGNGSGPRHINDSPFGTLPSSAE
PPLTALRPGGSEPPGLPTTGPRRRPGCSRKNRTRSHCRLGQAGSGASGT
GDAEGSGALPALACSLAPLGLALVLWTVLGPC

Full-length Nogo receptor-1 consists of a signal sequence, a N-terminus region (NT), eight leucine rich repeats (LRR), a LRRCT region (a leucine rich repeat domain C-terminal of the eight leucine rich repeats), a C-terminus region (CT) and a GPI anchor.

The NgR1 domain designations used herein are defined as follows:

TABLE 1
Example NgR1 domains

| | hNgR1 | rNgR1 | mNgR1 |
|---|---|---|---|
| Domain | (SEQ ID: 2) | (SEQ ID NO: 4) | (SEQ ID NO: 6) |
| Signal Seq. | 1-26 | 1-26 | 1-26 |
| LRRNT | 27-56 | 27-56 | 27-56 |
| LRR1 | 57-81 | 57-81 | 57-81 |
| LRR2 | 82-105 | 82-105 | 82-105 |
| LRR3 | 106-130 | 106-130 | 106-130 |
| LRR4 | 131-154 | 131-154 | 131-154 |
| LRR5 | 155-178 | 155-178 | 155-178 |
| LRR6 | 179-202 | 179-202 | 179-202 |
| LRR7 | 203-226 | 203-226 | 203-226 |
| LRR8 | 227-250 | 227-250 | 227-250 |
| LRRCT | 260-309 | 260-309 | 260-309 |
| CTS (CT | 310-445 | 310-445 | 310-445 |
| Signaling) | | | |
| GPI | 446-473 | 446-473 | 446-473 |

Soluble Nogo Receptor-1 Polypeptides

Some embodiments of the invention provide a soluble Nogo receptor-1 polypeptide. Soluble Nogo receptor-1 polypeptides of the invention comprise an NT domain; 8 LRRs and an LRRCT domain and lack a signal sequence and a functional GPI anchor (i.e., no GPI anchor or a GPI anchor that lacks the ability to efficiently associate to a cell membrane).

In some embodiments, a soluble Nogo receptor-1 polypeptide comprises a heterologous LRR. In some embodiments, a soluble Nogo receptor-1 polypeptide comprises 2, 3, 4, 5, 6, 7, or 8 heterologous LRRs. A heterologous LRR means an LRR obtained from a protein other than Nogo receptor-1. Exemplary proteins from which a heterologous LRR can be obtained are toll-like receptor (TLR1.2); T-cell activation leucine repeat rich protein; deceorin; OM-gp; insulin-like growth factor binding protein acidic labile subunit slit and robo; and toll-like receptor 4.

In some embodiments, the methods of the invention provide a soluble Nogo receptor-1 polypeptide of 319 amino acids (soluble Nogo receptor-1 344, sNogoR1-344, or sNogoR344) (residues 26-344 of SEQ ID NOs: 7 and 9 or residues 27-344 of SEQ ID NO: 9).

In some embodiments, the methods of the invention provide a soluble Nogo receptor-1 polypeptide of 285 amino acids (soluble Nogo receptor-1 310, sNogoR1-310, or sNogoR310) (residues 26-310 of SEQ ID NOs: 8 and 10 or residues 27-310 of SEQ ID NO: 10).

TABLE 2
Sequences of Human and Rat Nogo Receptor-1 Polypeptides

| | |
|---|---|
| SEQ ID NO: 7 | MKRASAGGSRLLAWVLWLQAWQVAAPCPGACVCYNEPK |
| (human 1-344) | VTTSCPQQGLQAVPVGIPAASQRIFLHGNRISHVPAASFRAC |
| | RNLTILWLHSNVLARIDAAAFTGLALLEQLDLSDNAQLRSV |
| | DPATFHGLGRLHTLHLDRCG_QELGPGLFRGLAALQYLYLQ |
| | DNALQALPDDTFRDLGNLTHLFLFIGNRISSVPERAFRGLHSL |
| | DRLLLHQNRVAHVHPHAFRDLGRLMTLYLFANNLSALPTE |
| | ALAPLRALQYLRLNDNPWVCDCRARPLWAWLQKFRGSSSE |
| | VPCSLPQRLAGRDLKRLAANDLQGCAVATGPYHPIWTGRA |
| | TDEEPLGLPKCCQPDAADKA |
| SEQ ID NO: 8 | MKRASAGGSRLLAWVLWLQAWQVAAPCPGACVCYNEPK |
| (human 1-310) | VTTSCPQQGLQAVPVGIPAASQRIFLPIGNRISHVPAASFRAC |
| | RNLTILWLHSNVLARIDAAAFTGLALLEQLDLSDNAQLRSV |
| | DPATFHGLGRLHTLHLDRCGLQELGPGLFRGLAALQYLYLQ |
| | DNALQALPDDTFRDLGNLTHLFLHGNRISSVPERAFRGLFISL |
| | DRLLLHQNRVAHVHPHAFRDLGRLMTLYLFANNLSALPTE |
| | ALAPLRALQYLRLNDNPWVCDCRARPLWAWLQKFRGSSSE |
| | VPCSLPQRLAGRDLKRLAANDLQGCA |
| SEQ ID NO: 9 | MKRASSGGSRLPTWVLWLQAWRVATPCPGACVCYNEPKV |
| (rat 1-344) | TTSRPQQGLQAVPAGIPASSQRIFLHGNRISYVPAASFQSCRN |
| | LTILWLHSNALAGIDAAAFTGLTLLEQLDLSDNAQLRVVDP |
| | TTFRGLGHLHTLHLDRCGLQELGPGLFRGLAALQYLYLQD |
| | NNLQALPDNTFRDLGNLTHLFLHGNRIPSVPEHAFRGLHSL |
| | DRULHQNHVARVHPHAFRDLGRLMTLYLFANNLSMLPAE |
| | VLVPLRSLQYLRLNDNPWVCDCRARPLWAWLQKFRGSSSG |
| | VPSNLPQRLAGRDLKRLATSDLEGCAVASGPFRPFQTNQLT |
| | DEELLGLPKCCQPDAADKA |
| SEQ ID NO: 10 | MKRASSGGSRLPTWVLWLQAWRVATPCPGACVCYNEPKV |
| (rat 1-310) | TTSRPQQGLQAVPAGIPASSQRIFLHGNRISYVPAASFQSCRN |
| | LTILWLHSNALAGIDAAAFTGLTLLEQLDLSDNAQLRVVDP |
| | TTFRGLGHLHTLHLDRCGLQELGPGLFRGLAALQYLYLQD |
| | NNLQALPDNTFRDLGNLTHLFLHGNRIPSVPEHAFRGLHSL |
| | DRLLLHQNHVARVHPHAFRDLGRLMTLYLFANNLSMLPAE |
| | VLVPLRSLQYLRLNDNPWVCDCRARPLWAWLQKFRGSSSG |
| | VPSNLPQRLAGRDLKRLATSDLEGCA |
| SEQ ID NO: 11 | MKRASAGGSRLLAWVLWLQAWQVAAPCPGACVCYNEPK |
| (human 1-310 | VTTSCPQQGLQAVPVGIPAASQRIFLHGNRISHVPAASFRAC |
| with ala | RNLTILWLHSNVLARIDAAAFTGLALLEQLDLSDNAQLRSV |
| substitutions at | DPATFHGLGRLHTLHLDRCGLQELGPGLFRGLAALQYLYLQ |
| amino acid | DNALQALPDDTFRDLGNLTHLFLHGNRISSVPERAFRGLHSL |
| positions 266 | DRLLLHQNRVAHVHPHAFRDLGRLMTLYLFANNLSALPTE |
| and 309) | ALAPLRALQYLRLNDNPWVCDARARPLWAWLQKFRGSSSE |
| | VPCSLPQRLAGRDLKRLAANDLQGAA |
| SEQ ID NO: 12 | MKRASSGGSRLPTWVLWLQAWRVATPCPGACVCYNEPKV |
| (rat 1-310 with | TTSRPQQGLQAVPAGIPASSQRIFLHGNRISYVPAASFQSCRN |
| ala substitutions | LTILWLHSNALAGIDAAAFTGLTLLEQLDLSDNAQLRVVDP |
| at amino acid | TTFRGLGHLHTLHLDRCGLQELGPGLFRGLAALQYLYLQD |
| positions 266 | NNLQALPDNTFRDLGNLTHLFLHGNRIPSVPEHAFRGLHSL |
| and 309) | DRLLLHQNHVARVHPHAFRDLGRLMTLYLFANNLSMLPAE |
| | VLVPLRSLQYLRLNDNPWVCDARARPLWAWLQKFRGSSSG |
| | VPSNLPQRLAGRDLKRLATSDLEGAA |
| SEQ ID NO: 13 | MKRASAGGSRLLAWVLWLQAWQVAAPCPGACVCYNEPK |
| (human 1-344 | VTTSCPQQGLQAVPVGIPAASQRIFLHGNRISHVPAASFRAC |
| with ala | RNLTILWLHSNVLARIDAAAFTGLALLEQLDLSDNAQLRSV |
| substitutions at | DPATFHGLGRLHTLHLDRCGLQELGPGLFRGLAALQYLYLQ |
| amino acid | DNALQALPDDTFRDLGNLTHLFLHGNRISSVPERAFRGLHSL |
| positions 266 | DRLLLHQNRVAHVHPHAFRDLGRLMTLYLFANNLSALPTE |
| and 309) | ALAPLRALQYLRLNDNPWVCDARARPLWAWLQKFRGSSSE |
| | VPCSLPQRLAGRDLKRLAANDLQGAAVATGPYHPIWTGRA |
| | TDEEPLGLPKCCQPDAADKA |

In some embodiments of the invention, peripheral administration of a soluble Nogo receptor-1 polypeptide of the invention increases the plasma to brain ratio of Aβ peptide in a mammal or enhances Aβ clearance from the brain of a mammal. In some embodiments of the invention, peripheral administration of a soluble Nogo receptor-1 polypeptide of the invention improves memory function or inhibits memory loss in a mammal. In some embodiments, the mammal is a human.

In some embodiments of the invention, the soluble Nogo receptor-1 polypeptide is 70%, 75%, 80%, 85%, 90%, or 95% identical to a reference amino acid sequence selected from the group consisting of: (i) amino acids 27 to 310 of SEQ ID NO:2; (ii) amino acids 27 to 344 of SEQ ID NO:2; (iii) amino acids 27 to 445 of SEQ ID NO:2; (iv) amino acids 27 to 309 of SEQ ID NO:2; (v) amino acids 1 to 310 of SEQ ID NO:2; (vi) amino acids 1 to 344 of SEQ ID NO:2; (vii) amino acids 1 to 445 of SEQ ID NO:2; (viii) amino acids 1 to 309 of SEQ ID NO:2; (ix) variants or derivatives of any of said reference amino acid sequences, and (x) a combination of one or more of said reference amino acid sequences or variants or derivatives thereof.

By “an NgR1 reference amino acid sequence,” or “reference amino acid sequence” is meant the specified sequence without the introduction of any amino acid substitutions. As one of ordinary skill in the art would understand, if there are no substitutions, the “isolated polypeptide” of the invention comprises an amino acid sequence which is identical to the reference amino acid sequence.

In some embodiments of the invention, the soluble Nogo receptor-1 polypeptide is selected from the group consisting of (i) amino acids a to 284 of SEQ ID NO:2, (ii) 210 to b of SEQ ID NO:2 and (iii) a to b of SEQ ID NO:2, wherein a is any integer from 200 to 210, and b is any integer from 284 to 295.

Soluble NgR1 polypeptides for use in the methods of the present invention include but are not limited to amino acids amino acids 27 to 310 of SEQ ID NO 2; amino acids 27 to 344 of SEQ ID NO:2, amino acids 27 to 445 of SEQ ID NO:2, amino acids 27 to 309 of SEQ ID NO:2, amino acids 1 to 310 of SEQ ID NO:2, amino acids 1 to 344 of SEQ ID NO:2, amino acids 1 to 445 of SEQ ID NO:2; and amino acids 1 to 309 of SEQ ID NO:2.

Any different amino acid may be substituted for an amino acid in the polypeptides of the invention. Amino acids that may be substituted in human NgR1 include but are not limited to those amino acids at positions 61; 92; 108; 122; 127; 131; 138; 139; 151; 176; 179; 227; 237; 250; 259; 108 and 131; 114 and 117; 127 and 151; 127 and 176; 143 and 144; 189 and 191; 196 and 199; 202 and 205 256 and 259; 267 and 269; 277 and 279; 114, 117 and 139; 189, 191 and 237; 189, 191, and 284; 202, 205 and 227; 202, 205 and 250; 220, 223 and 224; 237, 256 and 259; 296, 297 and 300; 171, 172, 175 and 176; 292, 296, 297 and 300; 196, 199, 220, 223 and 224; 171, 172, 175, 176, 196 and 199; 196, 199, 220, 223, 224 and 250; 108, 131 and 61; 36 and 38; 36, 38 and 61; 61, 131, 36 and 38; and 63 and 65. Which different amino acid is used depends on a number of criteria, for example, the effect of the substitution on the conformation of the polypeptide fragment, the charge of the polypeptide fragment, or the hydrophilicity of the polypeptide fragment. Amino acid substitutions for the amino acids of the polypeptides of the invention and the reference amino acid sequence can include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Typical amino acids to substitute for amino acids in the reference amino acid sequence include alanine, serine, threonine, in particular, alanine. Making such substitutions through engineering of a polynucleotide encoding the polypeptide fragment is well within the routine expertise of one of ordinary skill in the art.

A soluble NgR1 polypeptide can comprise a fragment of at least six, e.g., ten, fifteen, twenty, twenty-five, thirty, forty, fifty, sixty, seventy, one hundred, or more amino acids of SEQ ID NO:2. Corresponding fragments of soluble NgR1 polypeptides at least 70%, 75%, 80%, 85%, 90%, or 95% identical to a reference NgR1 polypeptide of SEQ ID NO:2 described herein are also contemplated.

As known in the art, “sequence identity” between two polypeptides is determined by comparing the amino acid sequence of one polypeptide to the sequence of a second polypeptide. When discussed herein, whether any particular polypeptide is at least about 70%, 75%, 80%, 85%, 90% or 95% identical to another polypeptide can be determined using methods and computer programs/software known in the art such as, but not limited to, the BESTFIT program (Wisconsin Sequence Analysis Package, Version 8 for Unix, Genetics Computer Group, University Research Park, 575 Science Drive, Madison, Wis. 53711). BESTFIT uses the local homology algorithm of Smith and Waterman, Advances in Applied Mathematics 2:482-489 (1981), to find the best segment of homology between two sequences. When using BESTFIT or any other sequence alignment program to determine whether a particular sequence is, for example, 95% identical to a reference sequence according to the present invention, the parameters are set, of course, such that the percentage of identity is calculated over the full length of the reference polypeptide sequence and that gaps in homology of up to 5% of the total number of amino acids in the reference sequence are allowed.

As discussed below in more detail, soluble NgR1 polypeptides for use in the methods of the present invention may include any combination of two or more soluble NgR1 polypeptides. Accordingly, soluble NgR1 polypeptide dimers, either homodimers or heterodimers, are contemplated. Two or more soluble NgR1 polypeptides as described herein may be directly connected, or may be connected via a suitable peptide linker. Such peptide linkers are described elsewhere herein.

NgR1 polypeptides for use in the methods disclosed herein can be composed of amino acids joined to each other by peptide bonds or modified peptide bonds, i.e., peptide isosteres, and may contain amino acids other than the 20 gene-encoded amino acids (e.g., non-naturally occurring amino acids). NgR1 polypeptides, may be modified by natural processes, such as posttranslational processing, or by chemical modification techniques which are well known in the art. Such modifications are well described in basic texts and in more detailed monographs, as well as in a voluminous research literature. Modifications can occur anywhere in the NgR1 polypeptide, including the peptide backbone, the amino acid side-chains and the amino or carboxyl termini, or on moieties such as carbohydrates. It will be appreciated that the same type of modification may be present in the same or varying degrees at several sites in a given NgR1 polypeptide. Also, a given NgR1 polypeptide may contain many types of modifications. NgR1 polypeptides may be branched, for example, as a result of ubiquitination, and they may be cyclic, with or without branching. Cyclic, branched, and branched cyclic NgR1 polypeptides may result from posttranslational natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, Proteins—Structure And Molecular Properties, T. E. Creighton, W. H. Freeman and Company, New York 2nd Ed., (1993); Posttranslational Covalent Modification Of Proteins, B. C. Johnson, Ed., Academic Press, New York, pgs. 1-12 (1983); Seifter et al., Meth Enzymol 182:626-646 (1990); Rattan et al., Ann NY Acad Sci 663:48-62 (1992)).

Polypeptides described herein may be cyclic. Cyclization of the polypeptides reduces the conformational freedom of linear peptides and results in a more structurally constrained molecule. Many methods of peptide cyclization are known in the art. For example, “backbone to backbone” cyclization by the formation of an amide bond between the N-terminal and the C-terminal amino acid residues of the peptide. The “backbone to backbone” cyclization method includes the formation of disulfide bridges between two α-thio amino acid residues (e.g., cysteine, homocysteine). Certain peptides of the present invention include modifications on the N- and C-terminus of the peptide to form a cyclic polypeptide. Such modifications include, but are not limited, to cysteine residues, acetylated cysteine residues, cysteine residues with a NH2 moiety and biotin. Other methods of peptide cyclization are described in Li & Roller, Curr. Top. Med. Chem. 3:325-341 (2002) and U.S Patent Publication No. U.S. 2005-0260626 A1, which are incorporated by reference herein in their entirety.

In methods of the present invention, an NgR1 polypeptide or polypeptide fragment of the invention can be administered directly as a preformed polypeptide, or indirectly through a nucleic acid vector. In some embodiments of the invention, an NgR1 polypeptide or polypeptide fragment of the invention is administered in a treatment method that includes: (1) transforming or transfecting an implantable host cell with a nucleic acid, e.g., a vector, that expresses an NgR1 polypeptide or polypeptide fragment of the invention; and (2) implanting the transformed host cell into a mammal, at the site of a disease, disorder or injury. In some embodiments of the invention, the implantable host cell is removed from a mammal, temporarily cultured, transformed or transfected with an isolated nucleic acid encoding an NgR1 polypeptide or polypeptide fragment of the invention, and implanted back into the same mammal from which it was removed. The cell can be, but is not required to be, removed from the same site at which it is implanted. Such embodiments, sometimes known as ex vivo gene therapy, can provide a continuous supply of the NgR1 polypeptide or polypeptide fragment of the invention, localized at the site of action, for a limited period of time.

Additional exemplary NgR polypeptides of the invention and methods and materials for obtaining these molecules for practicing the present invention are described below.

Fusion Proteins and Conjugated Polypeptides

Some embodiments of the invention involve the use of an NgR1 polypeptide that is not the full-length NgR1 protein, e.g., polypeptide fragments of NgR1, fused to a heterologous polypeptide moiety to form a fusion protein. Such fusion proteins can be used to accomplish various objectives, e.g., increased serum half-life, improved bioavailability, in vivo targeting to a specific organ or tissue type, improved recombinant expression efficiency, improved host cell secretion, ease of purification, and higher avidity. Depending on the objective(s) to be achieved, the heterologous moiety can be inert or biologically active. Also, it can be chosen to be stably fused to the NgR1 polypeptide moiety of the invention or to be cleavable, in vitro or in vivo. Heterologous moieties to accomplish these other objectives are known in the art.

In some embodiments, the soluble Nogo receptor-1 polypeptide of the invention is a component of a fusion protein that further comprises a heterologous polypeptide. In some embodiments, the heterologous polypeptide is an immunoglobulin constant domain. In some embodiments, the immunoglobulin constant domain is a heavy chain constant domain. In some embodiments, the heterologous polypeptide is an Fc fragment. In some embodiments the Fc is joined to the C-terminal end of the soluble Nogo receptor-1 polypeptide of the invention. In some embodiments the fusion Nogo receptor-1 protein is a dimer. The invention further encompasses variants, analogs, or derivatives of polypeptide fragments as described above.

In some embodiments of the invention, an NgR1 polypeptide fragment can be fused to one or more additional NgR polypeptide fragments, e.g., an NgR1, NgR2 or NgR3 polypeptide fragment along with Fc.

The human NgR2 polypeptide is shown below as SEQ ID NO:14.

Full-Length Human NgR2 (SEQ ID NO: 14):
MLPGLRRLLQ GPASACLLLT LLALPSVTPS CPMLCTCYSS
PPTVSCQANN FSSVPLSLPP STQRLFLQNN LIRSLRPGTF
GPNLLTLWLF SNNLSTIHPG TFRHLQALEE LDLGDNRHLR
SLEPDTFQGL ERLQSLHLYR CQLSSLPGNI FRGLVSLQYL
YLQENSLLHL QDDLFADLAN LSHLFLHGNR LRLLTEHVFR
GLGSLDRLLL HGNRLQGVHR AAFHGLSRLT ILYLFNNSLA
SLPGEALADL PALEFLRLNA NPWACDCRAR PLWAWFQRAR
VSSSDVTCAT PPERQGRDLR ALRDSDFQAC PPPTPTRPGS
RARGNSSSNH LYGVAEAGAP PADPSTLYRD LPAEDSRGRQ
GGDAPTEDDY WGGYGGEDQR GEQTCPGAAC QAPADSRGPA
LSAGLRTPLL CLLPLALHHL

The mouse NgR2 polypeptide is shown below as SEQ ID NO:15.

Full-Length Mouse NgR2 (SEQ ID NO: 15):
MLPGLRRLLQ GPASACLLLT LLALPSVTPS CPMLCTCYSS
PPTVSCQANN FSSVPLSLPP STQRLFLQNN LIRSLRPGTF
GPNLLTLWLF SNNLSTIHPG TFRHLQALEE LDLGDNRHLR
SLEPDTFQGL ERLQSLHLYR CQLSSLPGNI FRGLVSLQYL
YLQENSLLHL QDDLFADLAN LSHLFLHGNR LRLLTEHVFR
GLGSLDRLLL HGNRLQGVHR AAFHGLSRLT ILYLFNNSLA
SLPGEALADL PALEFLRLNA NPWACDCRAR PLWAWFQRAR
VSSSDVTCAT PPERQGRDLR ALRDSDFQAC PPPTPTRPGS
RARGNSSSNH LYGVAEAGAP PADPSTLYRD LPAEDSRGRQ
GGDAPTEDDY WGGYGGEDQR GEQTCPGAAC QAPADSRGPA
LSAGLRTPLL CLLPLALHHL

The human NgR3 polypeptide is shown below as SEQ ID NO:16.

Full-Length Human NgR3 (SEQ ID NO: 16):
MLRKGCCVEL LLLLVAAELP LGGGCPRDCV CYPAPMTVSC
QAHNFAAIPE GIPVDSERVF LQNNRIGLLQ PGHFSPAMVT
LWIYSNNITY IHPSTFEGFV HLEELDLGDN RQLRTLAPET
FQGLVKLHAL YLYKCGLSAL PAGVFGGLHS LQYLYLQDNH
IEYLQDDIFV DLVNLSHLFL HGNKLWSLGP GTFRGLVNLD
RLLLHENQLQ WVHHKAFHDL RRLTTLFLFN NSLSELQGEC
LAPLGALEFL RLNGNPWDCG CRARSLWEWL QRFRGSSSAV
PCVSPGLRHG QDLKLLRAED FRNCTGPASP HQIKSHTLTT
TDRAARKEHH SPHGPTRSKG HPHGPRPGHR KPGKNCTNPR
NRNQISKAGA GKQAPELPDY APDYQHKFSF DIMPTARPKR
KGKCARRTPI RAPSGVQQAS SASSLGASLL AWTLGLAVTL R

The mouse NgR3 polypeptide is shown below as SEQ ID NO:17.

Full-Length Mouse NgR3 (SEQ ID NO: 17):
MLRKGCCVEL LLLLLAGELP LGGGCPRDCV CYPAPMTVSC
QAHNFAAIPE GIPEDSERIF LQNNRITFLQ QGHFSPAMVT
LWIYSNNITF IAPNTFEGFV HLEELDLGDN RQLRTLAPET
FQGLVKLHAL YLYKCGLSAL PAGIFGGLHS LQYLYLQDNH
IEYLQDDIFV DLVNLSHLFL HGNKLWSLGQ GIFRGLVNLD
RLLLHENQLQ WVHHKAFHDL HRLTTLFLFN NSLTELQGDC
LAPLVALEFL RLNGNAWDCG CRARSLWEWL RRFRGSSSAV
PCATPELRQG QDLKLLRVED FRNCTGPVSP HQIKSHTLTT
SDRAARKEHH PSHGASRDKG HPHGHPPGSR SGYKKAGKNC
TSHRNRNQIS KVSSGKELTE LQDYAPDYQH KFSFDIMPTA
RPKRKGKCAR RTPIRAPSGV QQASSGTALG APLLAWILGL
AVTLR

In some embodiments of the methods of the invention, the soluble Nogo receptor polypeptide comprises a first polypeptide fragment and a second polypeptide fragment, wherein said first polypeptide fragment comprises an amino acid sequence identical to a first reference amino acid sequence, except for up to twenty individual amino acid substitutions, wherein said first reference amino acid sequence is selected from the group consisting of: (a) amino acids a to 445 of SEQ ID NO:2, (b) amino acids 27 to b of SEQ ID NO:2, and (c) amino acids a to b of SEQ ID NO:2, wherein a is any integer from 25 to 35, and b is any integer from 300 to 450; and wherein said second polypeptide fragment comprises an amino acid sequence identical to a second reference amino acid sequence, except for up to twenty individual amino acid substitutions, wherein said second reference amino acid sequence is selected from the group consisting of (a) amino acids c to 445 of SEQ ID NO:2, (b) amino acids 27 to d of SEQ ID NO:2, and (c) amino acids c to d of SEQ ID NO:2, wherein c is any integer from 25 to 35, and d is any integer from 300 to 450.

As an alternative to expression of an NgR fusion polypeptide, a chosen heterologous moiety can be preformed and chemically conjugated to the polypeptide. In most cases, a chosen heterologous moiety will function similarly, whether fused or conjugated to the NgR1 polypeptide. Therefore, in the following discussion of heterologous amino acid sequences, unless otherwise noted, it is to be understood that the heterologous sequence can be joined to the NgR polypeptide in the form of a fusion protein or as a chemical conjugate.

NgR polypeptides for use in the treatment methods disclosed herein include derivatives that are modified, i.e., by the covalent attachment of any type of molecule such that covalent attachment does not prevent the NgR polypeptide from performing its required function. For example, but not by way of limitation, the NgR polypeptides of the present invention may be modified e.g., by glycosylation, acetylation, pegylation, phosphylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

The heterologous polypeptide to which the NgR polypeptide is fused is useful therapeutically or is useful to target the NgR polypeptide NgR fusion proteins can be used to accomplish various objectives, e.g., increased serum half-life, improved bioavailability, in vivo targeting to a specific organ or tissue type, improved recombinant expression efficiency, improved host cell secretion, ease of purification, and higher avidity. Depending on the objective(s) to be achieved, the heterologous moiety can be inert or biologically active. Also, it can be chosen to be stably fused to the NgR polypeptide or to be cleavable, in vitro or in vivo. Heterologous moieties to accomplish these other objectives are known in the art.

Pharmacologically active polypeptides such as NgR polypeptides for use in the methods of the present invention may exhibit rapid in vivo clearance, necessitating large doses to achieve therapeutically effective concentrations in the body. In addition, polypeptides smaller than about 60 kDa potentially undergo glomerular filtration, which sometimes leads to nephrotoxicity. Fusion or conjugation of relatively small polypeptides such as polypeptide fragments of the NgR signaling domain can be employed to reduce or avoid the risk of such nephrotoxicity. Various heterologous amino acid sequences, i.e., polypeptide moieties or “carriers,” for increasing the in vivo stability, i.e., serum half-life, of therapeutic polypeptides are known. Examples include serum albumins such as, e.g., bovine serum albumin (BSA) or human serum albumin (HSA).

Due to its long half-life, wide in vivo distribution, and lack of enzymatic or immunological function, essentially full-length human serum albumin (HSA), or an HSA fragment, is commonly used as a heterologous moiety. Through application of methods and materials such as those taught in Yeh et al., Proc. Natl. Acad. Sci. USA, 89:1904-08 (1992) and Syed et al., Blood 89:3243-52 (1997), HSA can be used to form a fusion protein or polypeptide conjugate that displays pharmacological activity by virtue of the NgR polypeptide moiety while displaying significantly increased in vivo stability, e.g., 10-fold to 100-fold higher. The C-terminus of the HSA can be fused to the N-terminus of the NgR polypeptide moiety. Since HSA is a naturally secreted protein, the HSA signal sequence can be exploited to obtain secretion of the fusion protein into the cell culture medium when the fusion protein is produced in a eukaryotic, e.g., mammalian, expression system.

In certain embodiments, NgR polypeptides for use in the methods of the present invention further comprise a targeting moiety. Targeting moieties include a protein or a peptide which directs localization to a certain part of the body.

Some embodiments of the invention employ an NgR polypeptide moiety fused to a hinge and Fc region, i.e., the C-terminal portion of an Ig heavy chain constant region. In some embodiments, amino acids in the hinge region may be substituted with different amino acids. Exemplary amino acid substitutions for the hinge region according to these embodiments include substitutions of individual cysteine residues in the hinge region with different amino acids. Any different amino acid may be substituted for a cysteine in the hinge region. Amino acid substitutions for the amino acids of the polypeptides of the invention and the reference amino acid sequence can include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Typical amino acids to substitute for cysteines in the reference amino acid include alanine, serine, threonine, in particular, serine and alanine. Making such substitutions through engineering of a polynucleotide encoding the polypeptide fragment is well within the routine expertise of one of ordinary skill in the art.

Potential advantages of an NgR-polypeptide-Fc fusion include solubility, in vivo stability, and multivalency, e.g., dimerization. The Fc region used can be an IgA, IgD, or IgG Fc region (hinge-CH2-CH3). Alternatively, it can be an IgE or IgM Fc region (hinge-CH2-CH3-CH4). An IgG Fc region is generally used, e.g., an IgG1 Fc region or IgG4 Fc region. Materials and Methods for constructing and expressing DNA encoding Fc fusions are known in the art and can be applied to obtain fusions without undue experimentation. Some embodiments of the invention employ a fusion protein such as those described in Capon et al., U.S. Pat. Nos. 5,428,130 and 5,565,335.

The signal sequence is a polynucleotide that encodes an amino acid sequence that initiates transport of a protein across the membrane of the endoplasmic reticulum. Signal sequences useful for constructing an immunofusin include antibody light chain signal sequences, e.g., antibody 14.18 (Gillies et al., J. Immunol. Meth., 125:191-202 (1989)), antibody heavy chain signal sequences, e.g., the MOPC141 antibody heavy chain signal sequence (Sakano et al., Nature 286:5774 (1980)). Alternatively, other signal sequences can be used. See, e.g., Watson, Nucl. Acids Res. 12:5145 (1984). The signal peptide is usually cleaved in the lumen of the endoplasmic reticulum by signal peptidases. This results in the secretion of an immunofusin protein containing the Fc region and the NgR1 polypeptide moiety.

In some embodiments, the DNA sequence may encode a proteolytic cleavage site between the secretion cassette and the NgR1 polypeptide moiety. Such a cleavage, site may provide, e.g., for the proteolytic cleavage of the encoded fusion protein, thus separating the Fc domain from the target protein. Useful proteolytic cleavage sites include amino acid sequences recognized by proteolytic enzymes such as trypsin, plasmin, thrombin, factor Xa, or enterokinase K.

The secretion cassette can be incorporated into a replicable expression vector. Useful vectors include linear nucleic acids, plasmids, phagemids, cosmids and the like. An exemplary expression vector is pdC, in which the transcription of the immunofusin DNA is placed under the control of the enhancer and promoter of the human cytomegalovirus. See, e.g., Lo et al., Biochim. Biophys. Acta 1088:712 (1991); and Lo et al., Protein Engineering 11:495-500 (1998). An appropriate host cell can be transformed or transfected with a DNA that encodes an NgR1 polypeptide or polypeptide fragment of the invention and used for the expression and secretion of the polypeptide. Host cells that are typically used include immortal hybridoma cells, myeloma cells, 293 cells, Chinese hamster ovary (CHO) cells, Hela cells, and COS cells.

Fully intact, wild-type Fc regions display effector functions that normally are unnecessary and undesired in an Fc fusion protein used in the methods of the present invention. Therefore, certain binding sites typically are deleted from the Fc region during the construction of the secretion cassette. For example, since coexpression with the light chain is unnecessary, the binding site for the heavy chain binding protein, Bip (Hendershot et al., Immunol. Today 8:111-14 (1987)), is deleted from the CH2 domain of the Fc region of IgE, such that this site does not interfere with the efficient secretion of the immunofusin. Transmembrane domain sequences, such as those present in IgM, also are generally deleted.

The IgG1 Fc region is most often used. Alternatively, the Fc region of the other subclasses of immunoglobulin gamma (gamma-2, gamma-3 and gamma-4) can be used in the secretion cassette. The IgG1 Fc region of immunoglobulin gamma-1 is generally used in the secretion cassette and includes at least part of the hinge region, the CH2 region, and the CH3 region. In some embodiments, the Fc region of immunoglobulin gamma-1 is a CH2-deleted-Fc, which includes part of the hinge region and the CH3 region, but not the CH2 region. A CH2-deleted-Fc has been described by Gillies et al., Hum. Antibod. Hybridomas 1:47 (1990). In some embodiments, the Fc region of one of IgA, IgD, IgE, or IgM, is used.

NgR-polypeptide-moiety-Fc fusion proteins can be constructed in several different configurations. In one configuration, the C-terminus of the NgR polypeptide moiety is fused directly to the N-terminus of the Fc hinge moiety. In a slightly different configuration, a short polypeptide, e.g., 2-10 amino acids, is incorporated into the fusion between the N-terminus of the NgR polypeptide moiety and the C-terminus of the Fc moiety. In the alternative configuration, the short polypeptide is incorporated into the fusion between the C-terminus of the NgR polypeptide moiety and the N-terminus of the Fc moiety. An exemplary embodiment of this configuration is NgR1(310)-2XG4S-Fc, which is amino acids 26-310 of SEQ ID NO:2 linked to (Gly-Gly-Gly-Gly-Ser)₂ (SEQ ID NO:19) which is linked to Fc. Such a linker provides conformational flexibility, which may improve biological activity in some circumstances. If a sufficient portion of the hinge region is retained in the Fc moiety, the NgR-polypeptide-moiety-Fc fusion will dimerize, thus forming a divalent molecule. A homogeneous population of monomeric Fc fusions will yield monospecific, bivalent dimers. A mixture of two monomeric Fc fusions each having a different specificity will yield bispecific, bivalent dimers.

Any of a number of cross-linkers that contain a corresponding amino-reactive group and thiol-reactive group can be used to link an NgR polypeptide or polypeptide fragment of the invention to serum albumin. Examples of suitable linkers include amine reactive cross-linkers that insert a thiol-reactive maleimide, e.g., SMCC, AMAS, BMPS, MBS, EMCS, SMPB, SMPH, KMUS, and GMBS. Other suitable linkers insert a thiol-reactive haloacetate group, e.g., SBAP, SIA, SIAB. Linkers that provide a protected or non-protected thiol for reaction with sulfhydryl groups to product a reducible linkage include SPDP, SMPT, SATA, and SATP. Such reagents are commercially available (e.g., Pierce Chemical Company, Rockford, Ill.).

Conjugation does not have to involve the N-terminus of an NgR polypeptide or polypeptide fragment of the invention or the thiol moiety on serum albumin. For example, NgR-polypeptide-albumin fusions can be obtained using genetic engineering techniques, wherein the NgR polypeptide moiety is fused to the serum albumin gene at its N-terminus, C-terminus, or both.

NgR polypeptides of the invention can be fused to a polypeptide tag. The term “polypeptide tag,” as used herein, is intended to mean any sequence of amino acids that can be attached to, connected to, or linked to an NgR polypeptide and that can be used to identify, purify, concentrate or isolate the NgR polypeptide. The attachment of the polypeptide tag to the NgR polypeptide may occur, e.g., by constructing a nucleic acid molecule that comprises: (a) a nucleic acid sequence that encodes the polypeptide tag, and (b) a nucleic acid sequence that encodes an NgR polypeptide. Exemplary polypeptide tags include, e.g., amino acid sequences that are capable of being post-translationally modified, e.g., amino acid sequences that are biotinylated. Other exemplary polypeptide tags include, e.g., amino acid sequences that are capable of being recognized and/or bound by an antibody (or fragment thereof) or other specific binding reagent. Polypeptide tags that are capable of being recognized by an antibody (or fragment thereof) or other specific binding reagent include, e.g., those that are known in the art as “epitope tags.” An epitope tag may be a natural or an artificial epitope tag. Natural and artificial epitope tags are known in the art, including, e.g., artificial epitopes such as FLAG, Strep, or poly-histidine peptides. FLAG peptides include the sequence Asp-Tyr-Lys-Asp-Asp-Asp-Asp-Lys (SEQ ID NO:27) or Asp-Tyr-Lys-Asp-Glu-Asp-Asp-Lys (SEQ ID NO:28) (Einhauer, A. and Jungbauer, A., J. Biochem. Biophys. Methods 49:1-3:455-465 (2001)). The Strep epitope has the sequence Ala-Trp-Arg-His-Pro-Gln-Phe-Gly-Gly (SEQ ID NO:29). The VSV-G epitope can also be used and has the sequence Tyr-Thr-Asp-Ile-Glu-Met-Asn-Arg-Leu-Gly-Lys (SEQ ID NO:30). Another artificial epitope is a poly-His sequence having six histidine residues (His-His-His-His-His-His (SEQ ID NO:31). Naturally-occurring epitopes include the influenza virus hemagglutinin (HA) sequence Tyr-Pro-Tyr-Asp-Val-Pro-Asp-Tyr-Ala-Ile-Glu-Gly-Arg (SEQ ID NO:32) recognized by the monoclonal antibody 12CA5 (Murray et al., Anal. Biochem. 229:170-179 (1995)) and the eleven amino acid sequence from human c-myc (Myc) recognized by the monoclonal antibody 9E10 (Glu-Gln-Lys-Leu-Leu-Ser-Glu-Glu-Asp-Leu-Asn (SEQ ID NO:33) (Manstein et al., Gene 162:129-134 (1995)). Another useful epitope is the tripeptide Glu-Glu-Phe which is recognized by the monoclonal antibody YL 1/2. (Stammers et al. FEBS Lett. 283:298-302 (1991)).

In certain embodiments, the NgR polypeptide and the polypeptide tag may be connected via a linking amino acid sequence. As used herein, a “linking amino acid sequence” may be an amino acid sequence that is capable of being recognized and/or cleaved by one or more proteases. Amino acid sequences that can be recognized and/or cleaved by one or more proteases are known in the art. Exemplary amino acid sequences are those that are recognized by the following proteases: factor VIIa, factor IXa, factor Xa, APC, t-PA, u-PA, trypsin, chymotrypsin, enterokinase, pepsin, cathepsin B,H,L,S,D, cathepsin G, renin, angiotensin converting enzyme, matrix metalloproteases (collagenases, stromelysins, gelatinases), macrophage elastase, Cir, and Cis. The amino acid sequences that are recognized by the aforementioned proteases are known in the art. Exemplary sequences recognized by certain proteases can be found, e.g., in U.S. Pat. No. 5,811,252.

Polypeptide tags can facilitate purification using commercially available chromatography media.

In some embodiments of the invention, an NgR polypeptide fusion construct is used to enhance the production of an NgR polypeptide moiety in bacteria. In such constructs, a bacterial protein normally expressed and/or secreted at a high level is employed as the N-terminal fusion partner of an NgR1 polypeptide or polypeptide fragment of the invention. See, e.g., Smith et al., Gene 67:31 (1988); Hopp et al., Biotechnology 6:1204 (1988); La Vallie et al., Biotechnology 11:187 (1993).

By fusing an NgR polypeptide moiety at the amino and carboxy termini of a suitable fusion partner, bivalent or tetravalent forms of an NgR polypeptide or polypeptide fragment of the invention can be obtained. For example, an NgR polypeptide moiety can be fused to the amino and carboxy termini of an Ig moiety to produce a bivalent monomeric polypeptide containing two NgR polypeptide moieties. Upon dimerization of two of these monomers, by virtue of the Ig moiety, a tetravalent form of an NgR polypeptide is obtained. Such multivalent forms can be used to achieve increased binding affinity for the target. Multivalent forms of an NgR polypeptide or polypeptide fragment of the invention also can be obtained by placing NgR polypeptide moieties in tandem to form concatamers, which can be employed alone or fused to a fusion partner such as Ig or HSA.

Conjugated Polymers (Other than Polypeptides)

Some embodiments of the invention involve an NgR polypeptide or polypeptide fragment of the invention wherein one or more polymers are conjugated (covalently linked) to the NgR polypeptide. Examples of polymers suitable for such conjugation include polypeptides (discussed above), sugar polymers and polyalkylene glycol chains. Typically, but not necessarily, a polymer is conjugated to the NgR polypeptide or polypeptide fragment of the invention for the purpose of improving one or more of the following: solubility, stability, or bioavailability.

The class of polymer generally used for conjugation to an NgR polypeptide or polypeptide fragment of the invention is a polyalkylene glycol. Polyethylene glycol (PEG) is most frequently used. PEG moieties, e.g., 1, 2, 3, 4 or 5 PEG polymers, can be conjugated to each NgR polypeptide to increase serum half life, as compared to the NgR polypeptide alone. PEG moieties are non-antigenic and essentially biologically inert. PEG moieties used in the practice of the invention may be branched or unbranched.

The number of PEG moieties attached to the NgR polypeptide and the molecular weight of the individual PEG chains can vary. In general, the higher the molecular weight of the polymer, the fewer polymer chains attached to the polypeptide. Usually, the total polymer mass attached to an NgR polypeptide or polypeptide fragment is from 20 kDa to 40 kDa. Thus, if one polymer chain is attached, the molecular weight of the chain is generally 20-40 kDa. If two chains are attached, the molecular weight of each chain is generally 10-20 kDa. If three chains are attached, the molecular weight is generally 7-14 kDa.

The polymer, e.g., PEG, can be linked to the NgR polypeptide through any suitable, exposed reactive group on the polypeptide. The exposed reactive group(s) can be, e.g., an N-terminal amino group or the epsilon amino group of an internal lysine residue, or both. An activated polymer can react and covalently link at any free amino group on the NgR polypeptide. Free carboxylic groups, suitably activated carbonyl groups, hydroxyl, guanidyl, imidazole, oxidized carbohydrate moieties and mercapto groups of the NgR polypeptide (if available) also can be used as reactive groups for polymer attachment.

In a conjugation reaction, from about 1.0 to about 10 moles of activated polymer per mole of polypeptide, depending on polypeptide concentration, is typically employed. Usually, the ratio chosen represents a balance between maximizing the reaction while minimizing side reactions (often non-specific) that can impair the desired pharmacological activity of the NgR polypeptide moiety. Preferably, at least 50% of the biological activity (as demonstrated, e.g., in any of the assays described herein or known in the art) of the NgR polypeptide is retained, and most preferably nearly 100% is retained.

The polymer can be conjugated to the NgR polypeptide using conventional chemistry. For example, a polyalkylene glycol moiety can be coupled to a lysine epsilon amino group of the NgR polypeptide. Linkage to the lysine side chain can be performed with an N-hydroxylsuccinimide (NHS) active ester such as PEG succinimidyl succinate (SS-PEG) and succinimidyl propionate (SPA-PEG). Suitable polyalkylene glycol moieties include, e.g., carboxymethyl-NHS and norleucine-NHS, SC. These reagents are commercially available. Additional amine-reactive PEG linkers can be substituted for the succinimidyl moiety. These include, e.g., isothiocyanates, nitrophenylcarbonates (PNP), epoxides, benzotriazole carbonates, SC-PEG, tresylate, aldehyde, epoxide, carbonylimidazole and PNP carbonate. Conditions are usually optimized to maximize the selectivity and extent of reaction. Such optimization of reaction conditions is within ordinary skill in the art.

PEGylation can be carried out by any of the PEGylation reactions known in the art. See, e.g., Focus on Growth Factors, 3: 4-10, 1992 and European patent applications EP 0 154 316 and EP 0 401 384. PEGylation may be carried out using an acylation reaction or an alkylation reaction with a reactive polyethylene glycol molecule (or an analogous reactive water-soluble polymer).

PEGylation by acylation generally involves reacting an active ester derivative of polyethylene glycol. Any reactive PEG molecule can be employed in the PEGylation. PEG esterified to N-hydroxysuccinimide (NHS) is a frequently used activated PEG ester. As used herein, “acylation” includes without limitation the following types of linkages between the therapeutic protein and a water-soluble polymer such as PEG: amide, carbamate, urethane, and the like. See, e.g., Bioconjugate Chem. 5: 133-140, 1994. Reaction parameters are generally selected to avoid temperature, solvent, and pH conditions that would damage or inactivate the NgR polypeptide.

Generally, the connecting linkage is an amide and typically at least 95% of the resulting product is mono-, di- or tri-PEGylated. However, some species with higher degrees of PEGylation may be formed in amounts depending on the specific reaction conditions used. Optionally, purified PEGylated species are separated from the mixture, particularly unreacted species, by conventional purification methods, including, e.g., dialysis, salting-out, ultrafiltration, ion-exchange chromatography, gel filtration chromatography, hydrophobic exchange chromatography, and electrophoresis.

PEGylation by alkylation generally involves reacting a terminal aldehyde derivative of PEG with an NgR1 polypeptide or polypeptide fragment of the invention in the presence of a reducing agent. In addition, one can manipulate the reaction conditions to favor PEGylation substantially only at the N-terminal amino group of the NgR polypeptide, i.e. a mono-PEGylated protein. In either case of mono-PEGylation or poly-PEGylation, the PEG groups are typically attached to the protein via a —CH2—NH— group. With particular reference to the —CH2— group, this type of linkage is known as an “alkyl” linkage.

Derivatization via reductive alkylation to produce an N-terminally targeted mono-PEGylated product exploits differential reactivity of different types of primary amino groups (lysine versus the N-terminal) available for derivatization. The reaction is performed at a pH that allows one to take advantage of the pKa differences between the epsilon-amino groups of the lysine residues and that of the N-terminal amino group of the protein. By such selective derivatization, attachment of a water-soluble polymer that contains a reactive group, such as an aldehyde, to a protein is controlled: the conjugation with the polymer takes place predominantly at the N-terminus of the protein and no significant modification of other reactive groups, such as the lysine side chain amino groups, occurs.

The polymer molecules used in both the acylation and alkylation approaches are selected from among water-soluble polymers. The polymer selected is typically modified to have a single reactive group, such as an active ester for acylation or an aldehyde for alkylation, so that the degree of polymerization may be controlled as provided for in the present methods. An exemplary reactive PEG aldehyde is polyethylene glycol propionaldehyde, which is water stable, or mono C₁-C₁₀ alkoxy or aryloxy derivatives thereof (see, e.g., Harris et al., U.S. Pat. No. 5,252,714). The polymer may be branched or unbranched. For the acylation reactions, the polymer(s) selected typically have a single reactive ester group. For reductive alkylation, the polymer(s) selected typically have a single reactive aldehyde group. Generally, the water-soluble polymer will not be selected from naturally occurring glycosyl residues, because these are usually made more conveniently by mammalian recombinant expression systems.

Methods for preparing a PEGylated NgR polypeptides of the invention generally includes the steps of (a) reacting an NgR1 polypeptide or polypeptide fragment of the invention with polyethylene glycol (such as a reactive ester or aldehyde derivative of PEG) under conditions whereby the molecule becomes attached to one or more PEG groups, and (b) obtaining the reaction product(s). In general, the optimal reaction conditions for the acylation reactions will be determined case-by-case based on known parameters and the desired result. For example, a larger the ratio of PEG to protein, generally leads to a greater the percentage of poly-PEGylated product.

Reductive alkylation to produce a substantially homogeneous population of mono-polymer/NgR polypeptide generally includes the steps of: (a) reacting an NgR1 polypeptide or polypeptide fragment of the invention with a reactive PEG molecule under reductive alkylation conditions, at a pH suitable to permit selective modification of the N-terminal amino group of NgR; and (b) obtaining the reaction product(s).

For a substantially homogeneous population of mono-polymer/NgR polypeptide, the reductive alkylation reaction conditions are those that permit the selective attachment of the water-soluble polymer moiety to the N-terminus of a NgR polypeptide or polypeptide fragment of the invention. Such reaction conditions generally provide for pKa differences between the lysine side chain amino groups and the N-terminal amino group. For purposes of the present invention, the pH is generally in the range of 3-9, typically 3-6.

NgR polypeptides of the invention can include a tag, e.g., a moiety that can be subsequently released by proteolysis. Thus, the lysine moiety can be selectively modified by first reacting a His-tag modified with a low-molecular-weight linker such as Traut's reagent (Pierce Chemical Company, Rockford, Ill.) which will react with both the lysine and N-terminus, and then releasing the His tag. The polypeptide will then contain a free SH group that can be selectively modified with a PEG containing a thiol-reactive head group such as a maleimide group, a vinylsulfone group, a haloacetate group, or a free or protected SH.

Traut's reagent can be replaced with any linker that will set up a specific site for PEG attachment. For example, Traut's reagent can be replaced with SPDP, SMPT, SATA, or SATP (Pierce Chemical Company, Rockford, Ill.). Similarly one could react the protein with an amine-reactive linker that inserts a maleimide (for example SMCC, AMAS, BMPS, MBS, EMCS, SMPB, SMPH, KMUS, or GMBS), a haloacetate group (SBAP, SIA, SIAB), or a vinylsulfone group and react the resulting product with a PEG that contains a free SH.

In some embodiments, the polyalkylene glycol moiety is coupled to a cysteine group of the NgR polypeptide. Coupling can be effected using, e.g., a maleimide group, a vinylsulfone group, a haloacetate group, or a thiol group.

Optionally, the NgR polypeptide is conjugated to the polyethylene-glycol moiety through a labile bond. The labile bond can be cleaved in, e.g., biochemical hydrolysis, proteolysis, or sulfhydryl cleavage. For example, the bond can be cleaved under in vivo (physiological) conditions.

The reactions may take place by any suitable method used for reacting biologically active materials with inert polymers, generally at about pH 5-8, e.g., pH 5, 6, 7, or 8, if the reactive groups are on the alpha amino group at the N-terminus. Generally the process involves preparing an activated polymer and thereafter reacting the protein with the activated polymer to produce the soluble protein suitable for formulation.

Nucleic Acid Molecules of the Present Invention

The human Nogo receptor-1 polynucleotide is shown below as SEQ ID NO:1.

Full-Length Human Nogo receptor-1 is encoded by
nucleotide 166 to nucleotide 1587 of SEQ ID NO: 1:
agcccagcca gagccgggcg gagcggagcg cgccgagcct
cgtcccgcgg ccgggccggg gccgggccgt agcggcggcg
cctggatgcg gacccggccg cggggagacg ggcgcccgcc
ccgaaacgac tttcagtccc cgacgcgccc cgcccaaccc
ctacgatgaa gagggcgtcc gctggaggga gccggctgct
ggcatgggtg ctgtggctgc aggcctggca ggtggcagcc
ccatgcccag gtgcctgcgt atgctacaat gagcccaagg
tgacgacaag ctgcccccag cagggcctgc aggctgtgcc
cgtgggcatc cctgctgcca gccagcgcat cttcccgcac
ggcaaccgca tctcgcatgt gccagctgcc agcctccgtg
cctgccgcaa cctcaccatc ctgtggctgc actcgaatgt
gccggcccga actgatgcgg ctgcctccac cggcctggcc
ctcctggagc agctggacct cagcgacaat gcacagctcc
ggtctgtgga ccctgccaca ttccacggcc tgggccgcct
acacacgctg cacctggacc gctgcggcct gcaggagctg
ggcccggggc tgtcccgcgg cctggcCgcc ctgcagtacc
cctacctgca ggacaacgcg ctgcaggcac tgcctgatga
caccccccgc gacctgggca acctcacaca cctcttcctg
cacggcaacc gcatccccag cgtgcccgag cgcgccttcc
gtgggctgca cagcctcgac cgtctcctac tgcaccagaa
ccgcgtggcc catgcgcacc cgcatgcctc ccgcgacctc
ggccgcctca tgacactcta tctgtttgcc aacaatctat
cagcgctgcc cactgaggcc ccggcccccc tgcgtgccct
gcagtacctg aggctcaacg acaacccctg ggtgtgtgac
cgccgggcac gcccactctg ggcctggctg cagaagttcc
gcggctcctc ctccgaggtg ccctgcagcc tcccgcaacg
cctggctggc cgtgacctca aacgcctagc cgccaatgac
ctgcagggct gcgctgtggc caccggccct taccatccca
cctggaccgg cagggccacc gatgaggagc cgctggggct
tcccaagtgc tgccagccag atgccgccga caaggcccca
gtactggagc ctggaagacc agcttcggca ggcaatgcgc
tgaagggacg cgtgccgccc ggtgacagcc cgccgggcaa
cggctctggc ccacggcaca tcaatgaccc accctttggg
accccgcctg gctctgccga gcccccgctc actgcagtgc
ggcccgaggg ctccgagcca ccagggttcc ccacctcggg
ccctcgccgg aggccaggct gttcacgcaa gaaccgcacc
cgcagccact gccgcctggg ccaggcaggc agcgggggtg
gcgggactgg tgactcagaa ggctcaggtg ccctacccag
cctcacctgc agcctcaccc ccctgggcct ggcgctggtg
ctgtggacag tgcttgggcc ctgctgaccc ccagcggaca
caagagcgcg ctcagcagcc aggtgtgtgt acatacgggg
tctctctcca cgccgccaag ccagccgggc ggccgacccg
tggggcaggc caggccaggt cccccctgat ggacgcccg

The rat Nogo receptor-1 polynucleotide is shown below as SEQ ID NO:3.

atgaagaggg cgccctccgg aggaagccgg ctgccgacat
gggcgccacg gctacaggcc tggagggtag caacgccctg
ccctggtgcc tgtgtgtgct acaatgagcc caaggtcaca
acaagccgcc cccagcaggg cctgcaggct gtacccgctg
gcatcccagc ctccagccag agaatcttcc tgcacggcaa
ccgaatctct tacgtgccag ccgccagctt ccagtcatgc
cggaatctca ccatcctgtg gctgcactca aatgcgctgg
ccgggattga tgccgcggcc ttcactggtc tgaccctcct
ggagcaacta gatcttagtg acaatgcaca gctccgtgtc
gtggacccca ccacgttccg tggcctgggc cacctgcaca
cgctgcacct agaccgatgc ggcctgcagg agctggggcc
tggcctattc cgtgggctgg cagctctgca gtacctctac
ctacaagaca acaacctgca ggcacttccc gacaacacct
tccgagacct gggcaacctc acgcatctct ttctgcatgg
caaccgtatc cccagtgttc ctgagcacgc tttccgtggc
ttgcacagtc ttgaccgtct cctcttgcac cagaaccatg
tggctcgtgt gcacccacat gccttccggg accttggccg
actcatgacc ctctacctgt ttgccaacaa cctctccatg
ctaaaagcag aggtcctagt gcccctgagg tctctgcagt
acctgcgact caatgacaac ccctgggtgt gtgactgcag
ggcacgtccg ctctgggcct ggctgcagaa gttccgaggt
tcctcatccg gggtgcccag caacctaccc caacgcctgg
caggccgtga tctgaagcgc ctggctacca gtgacttaga
gggttgtgct gtggcttcgg ggcccttccg tcccttccag
accaatcagc tcactgatga ggagctgctg ggcctcccca
agtgctgcca gccggatgct gcagacaagg cctcagtact
ggaacccggg aggccggcgt ctgttggaaa tgcactcaag
ggacgtgtgc ctcccggtga cactccacca ggcaatggct
caggcccacg gcacatcaat gactctccat ttgggacttt
gcccggctct gcagagcccc cactgactgc cctgcggcct
gggggttccg agcccccggg actgcccacc acgggccccc
gcaggaggcc aggttgttcc agaaagaacc gcacccgtag
ccactgccgt ctgggccagg caggaagtgg gagcagtgga
actggggatg cagaaggttc gggggccctg cctgccctgg
cctgcagcct tgctcctctg ggccttgcac tggtactttg
gacagtgctt gggccctgct ga

The mouse Nogo receptor-1 polynucleotide is shown below as SEQ ID NO:5.

Full-Length Mouse Nogo receptor-1 is encoded by
nucleotide 178 to nucleotide 1599 of SEQ ID NO: 5:
agccgcagcc cgcgagccca gcccggcccg gtagagcgga
gcgccggagc ctcgtcccgc ggccgggccg ggaccgggcc
ggagcagcgg cgcctggatg cggacccggc cgcgcgcaga
cgggcgcccg ccccgaagcc gcttccagtg cccgacgcgc
cccgctcgac cccgaagatg aagagggcgt cctccggagg
aagcaggctg ctggcatggg tgttatggct acaggcctgg
agggtagcaa caccatgccc tggtgctcgc gtgtgctaca
atgagcccaa ggtaacaaca agctgccccc agcagggtct
gcaggctgtg cccactggca ccccagcctc tagccagcga
atctccctgc atggcaaccg aatctctcac gtgccagctg
cgagcttcca gccatgccga aatctcacta tcctgtggct
gcactctaat gcgctggctc ggatcgacgc tgctgccttc
actggtctga ccctcccgga gcaactagac cttagtgata
atgcacagct tcatgtcgtg gaccctacca cgttccacgg
cctgggccac ctgcacacac tgcacctaga ccgatgtggc
ctgcgggagc tgggtcccgg cctattccgt ggactagcag
ctctgcagta cctctaccta caagacaaca atctgcaggc
actccctgac aacacctttc gagacctggg caacctcacg
catctctttc tgcatggcaa ccgtatcccc agtgtgcctg
agcacgcttt ccgtggcctg cacagtcttg accgcctcct
cttgcaccag aaccatgtgg ctcgtgtgca cccacatgcc
ttccgggacc ttggccgcct catgaccctc tacctgtttg
ccaacaacct ctccatgctg cctgcagagg tcctaatgcc
cctgaggtct ctgcagtacc tgcgactcaa tgacaacccc
tgggtgtgtg actgccgggc acgtccactc tgggcctggc
tgcagaagtt ccgaggttcc tcatcagagg tgccctgcaa
cctgccccaa cgcctggcag accgtgacct taagcgcctc
gctgccagtg acctagaggg ctgtgctgtg gcttcaggac
ccttccgtcc catccagacc agtcagctca ctgatgagga
gctgctgagt ctccccaagt gctgccagcc agatgctgca
gacaaagcct cagtactgga acccgggagg ccagcttctg
ccggaaacgc cctcaaggga cgtgtgcctc ccggtgacac
tccaccaggc aatggctcag gccctcggca catcaatgac
tctccatttg gaactttgcc cagctctgca gagcccccac
tgactgccct gcggcctggg ggttccgagc caccaggact
tcccaccact ggtccccgca ggaggccagg ttgttcccgg
aagaatcgca cccgcagcca ctgccgtctg ggccaggcgg
gaagtggggc cagtggaaca ggggacgcag agggttcagg
ggctctgcct gctctggcct gcagccttgc tcctctgggc
cttgcactgg tactttggac agtgcttggg ccctgctgac
cagccaccag ccaccaggtg tgtgtacata tggggtctcc
ctccacgccg ccagccagag ccagggacag gctctgaggg
gcaggccagg ccctccctga cagatgcctc cccaccagcc
cacccccatc tccaccccat catgtttaca gggttccggg
ggtggcgttt gttccagaac gccacctccc acccggatcg
cggtatatag agatatgaat cctattttac ttgtgtaaaa
tatcggatga cgtggaataa agagctctct tcttaaaaaa
aaaaaaaaaa aa

The present invention provide a polynucleotide that encodes any of the recited polypeptides or polypeptide fragments of the invention.

In some embodiments, the nucleic acid encodes a polypeptide selected from the group consisting of amino acid residues 26-344 of Nogo receptor-1 as shown in SEQ ID NOs: 7 and 9 or amino acid residues 27-344 of Nogo receptor-1 as shown in SEQ ID NO: 9. In some embodiments, the nucleic acid molecule encodes a polypeptide selected from the group consisting of amino acid residues 26-310 of Nogo receptor-1 as shown in SEQ ID NOs: 8 and 10 or amino acid residues 27-310 of Nogo receptor-1 as shown in SEQ ID NO: 10. As used herein, “nucleic acid” means genomic DNA, cDNA, mRNA and antisense molecules, as well as nucleic acids based on alternative backbones or including alternative bases whether derived from natural sources or synthesized. In some embodiments, the nucleic acid further comprises a transcriptional promoter and optionally a signal sequence each of which is operably linked to the nucleotide sequence encoding the polypeptides of the invention.

In some embodiments, the invention provides a nucleic acid encoding a Nogo receptor-1 fusion protein of the invention, including a fusion protein comprising a polypeptide selected from the group consisting of amino acid residues 26-344 of Nogo receptor-1 as shown in SEQ ID NOs: 7 and 9 or amino acid residues 27-344 of SEQ ID NO: 9 and amino acid residues 26-310 of Nogo receptor-1 as shown in SEQ ID NOs: 8 and 10 or amino acid residues 27-310 of SEQ ID NO: 10. In some embodiments, the nucleic acid encoding a Nogo receptor-1 fusion protein further comprises a transcriptional promoter and optionally a signal sequence. In some embodiments, the nucleotide sequence further encodes an immunoglobulin constant region. In some embodiments, the immunoglobulin constant region is a heavy chain constant region. In some embodiments, the nucleotide sequence further encodes an immunoglobulin heavy chain constant region joined to a hinge region. In some embodiments the nucleic acid further encodes Fc. In some embodiments the Nogo receptor-1 fusion proteins comprise an Fc fragment.

The encoding nucleic acids of the present invention may further be modified so as to contain a detectable label for diagnostic and probe purposes. A variety of such labels are known in the art and can readily be employed with the encoding molecules herein described. Suitable labels include, but are not limited to, biotin, radiolabeled nucleotides and the like. A skilled artisan can employ any of the art known labels to obtain a labeled encoding nucleic acid molecule.

The present invention also includes polynucleotides that hybridize under moderately stringent or high stringency conditions to the noncoding strand, or complement, of a polynucleotide that encodes any one of the polypeptides of the invention. In some embodiments, polynucleotides that hybridize encode a polypeptide of the invention. Stringent conditions are known to those skilled in the art and can be found in CURRENT PROTOCOLS IN MOLECULAR BIOLOGY, John Wiley & Sons, N.Y. (1989), 6.3.1-6.3.6.

Compositions

In some embodiments, the invention provides compositions comprising a soluble Nogo receptor polypeptide or fusion protein of the present invention.

In some embodiments, the invention provides a composition comprising a polynucleotide of the present invention.

In some embodiments, the present invention may contain suitable pharmaceutically acceptable carriers comprising excipients and auxiliaries which facilitate processing of the active compounds into preparations which can be used pharmaceutically for delivery to the site of action. Suitable formulations for parenteral administration include aqueous solutions of the active compounds in water-soluble form, for example, water-soluble salts. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides. Aqueous injection suspensions may contain substances which increase the viscosity of the suspension include, for example, sodium carboxymethyl cellulose, sorbitol and dextran. Optionally, the suspension may also contain stabilizers. Liposomes can also be used to encapsulate the molecules of this invention for delivery into the cell. Exemplary “pharmaceutically acceptable carriers” are any and all solvents, dispersion media, coatings, antibacterial and antifungal agents, isotonic and absorption delaying agents, and the like that are physiologically compatible, water, saline, phosphate buffered saline, dextrose, glycerol, ethanol and the like, as well as combinations thereof. In some embodiments, the composition comprises isotonic agents, for example, sugars, polyalcohols such as mannitol, sorbitol, or sodium chloride. In some embodiments, the compositions comprise pharmaceutically acceptable substances such as wetting or minor amounts of auxiliary substances such as wetting or emulsifying agents, preservatives or buffers, which enhance the shelf life or effectiveness of the soluble Nogo receptors or fusion proteins of the invention.

Compositions of the invention may be in a variety of forms, including, for example, liquid, semi-solid and solid dosage forms, such as liquid solutions (e.g., injectable and infusible solutions), dispersions or suspensions. The preferred form depends on the intended mode of administration and therapeutic application. In one embodiment, compositions are in the form of injectable or infusible solutions, such as compositions similar to those used for passive immunization of humans with other antibodies.

The composition can be formulated as a solution, micro emulsion, dispersion, liposome, or other ordered structure suitable to high drug concentration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

In some embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See e.g., Sustained and Controlled Release Drug Delivery Systems, J. R. Robinson, ed., Marcel Dekker, Inc., New York (1978).

The pharmaceutical compositions of the invention may include a “therapeutically effective amount” or a “prophylactically effective amount” of a polypeptide(s), or fusion protein of the invention. A “therapeutically effective amount” refers to an amount effective, at dosages and for periods of time necessary, to achieve the desired therapeutic result. A therapeutically effective amount of the soluble Nogo receptor polypeptide or Nogo receptor fusion protein may vary according to factors such as the disease state, age, sex, and weight of the individual. A therapeutically effective amount is also one in which any toxic or detrimental effects of the soluble Nogo receptor polypeptide or Nogo receptor fusion protein are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage unit form as used herein refers to physically discrete units suited as unitary dosages for the mammalian subjects to be treated, each unit containing a predetermined quantity of active compound calculated to produce the desired therapeutic effect in association with the required pharmaceutical carrier. The specification for the dosage unit forms of the invention are dictated by and directly dependent on (a) the unique characteristics of the soluble receptor polypeptide or Nogo receptor fusion protein and the particular therapeutic or prophylactic effect to be achieved, and (b) the limitations inherent in the art of compounding such a soluble receptor polypeptide or Nogo receptor fusion protein for the treatment of sensitivity in individuals. In some embodiments a therapeutically effective dose range for the soluble Nogo receptor polypeptide 0.001-10 mg/Kg per day. In some embodiments a therapeutically effective dose range for soluble Nogo receptor polypeptides thereof is 0.01-1 mg/Kg per day. In some embodiments a therapeutically effective dose range for the Nogo receptor polypeptides thereof is 0.05-0.5 mg/Kg per day.

For treatment with a soluble NgR1 receptor polypeptide of the invention, the dosage can range, e.g., from about 0.0001 to 100 mg/kg, and more usually 0.01 to 5 mg/kg (e.g., 0.02 mg/kg, 0.25 mg/kg, 0.5 mg/kg, 0.75 mg/kg, 1 mg/kg, 2 mg/kg, etc.), of the host body weight. For example dosages can be 1 mg/kg body weight or 10 mg/kg body weight or within the range of 1-10 mg/kg, preferably at least 1 mg/kg. Doses intermediate in the above ranges are also intended to be within the scope of the invention. Subjects can be administered such doses daily, on alternative days, weekly or according to any other schedule determined by empirical analysis. An exemplary treatment entails administration in multiple dosages over a prolonged period, for example, of at least six months. Additional exemplary treatment regimes entail administration once per every two weeks or once a month or once every 3 to 6 months. Exemplary dosage schedules include 1-10 mg/kg or 15 mg/kg on consecutive days, 30 mg/kg on alternate days or 60 mg/kg weekly.

In some methods, two or more soluble NgR1 receptor polypeptides or fusion proteins are administered simultaneously, in which case the dosage of each polypeptide or fusion protein administered falls within the ranges indicated. Supplementary active compounds also can be incorporated into the compositions used in the methods of the invention. For example, a NgR polypeptide or fusion protein may be coformulated with and/or coadministered with one or more additional therapeutic agents, such as an adrenergic, anti-adrenergic, anti-androgen, anti-anginal, anti-anxiety, anticonvulsant, antidepressant, anti-epileptic, antihyperlipidemic, antihyperlipoproteinemic, antihypertensive, anti-inflammatory, antiobessional, antiparkinsonian, antipsychotic, adrenocortical steroid; adrenocortical suppressant; aldosterone antagonist; amino acid; anabolic steroid; analeptic; androgen; blood glucose regulator; cardioprotectant; cardiovascular; cholinergic agonist or antagonist; cholinesterase deactivator or inhibitor, cognition adjuvant or enhancer; dopaminergic; enzyme inhibitor, estrogen, free oxygen radical scavenger; GABA agonist; glutamate antagonist; hormone; hypocholesterolemic; hypolipidemic; hypotensive; immunizing; immunostimulant; monoamine oxidase inhibitor, neuroprotective; NMDA antagonist; AMPA antagonist, competitive or-non-competitive NMDA antagonist; opioid antagonist; potassium channel opener; non-hormonal sterol derivative; post-stroke and post-head trauma treatment; prostaglandin; psychotropic; relaxant; sedative; sedative-hypnotic; selective adenosine antagonist; serotonin antagonist; serotonin inhibitor; selective serotonin uptake inhibitor; serotonin receptor antagonist; sodium and calcium channel blocker; steroid; stimulant; and thyroid hormone and inhibitor agents.

In embodiments of the present invention, the NgR polypeptide or fusion protein is delivered peripheral to the central nervous system. “Peripheral to the central nervous system” includes any route of administration except for those routes of administration wherein the NgR polypeptide is administered directly to the central nervous system, e.g., intracerebroventricularly, or intrathecally.

In some embodiments, the NgR polypeptide or fusion protein is administered by a route selected from the group consisting of oral administration; nasal administration; parenteral administration; transdermal administration; topical administration; intraocular administration; intrabronchial administration; intraperitoneal administration; intravenous administration; subcutaneous administration; intramuscular administration; and a combination of two or more of these routes of administration. In one embodiment, the NgR polypeptide or fusion protein is administered subcutaneously.

Parenteral injectable administration is generally used for subcutaneous, intramuscular or intravenous injections and infusions. Additionally, one approach for parenteral administration employs the implantation of a slow-release or sustained-released systems, which assures that a constant level of dosage is maintained, according to U.S. Pat. No. 3,710,795, incorporated herein by reference in its entirety.

The invention encompasses any suitable delivery method for a NgR polypeptide or fusion protein to a selected target tissue, including bolus injection of an aqueous solution or implantation of a controlled-release system. Use of a controlled-release implant reduces the need for repeat injections.

The compositions may also comprise a NgR polypeptide or fusion protein of the invention dispersed in a biocompatible carrier material that functions as a suitable delivery or support system for the compounds. Suitable examples of sustained release carriers include semipermeable polymer matrices in the form of shaped articles such as suppositories or capsules. Implantable or microcapsular sustained release matrices include polylactides (U.S. Pat. No. 3,773,319; EP 58,481), copolymers of L-glutamic acid and gamma-ethyl-L-glutamate (Sidman et al., Biopolymers 22:547-56 (1985)); poly(2-hydroxyethyl-methacrylate), ethylene vinyl acetate (Langer et al., J. Biomed. Mater. Res. 15:167-277 (1981); Langer, Chem. Tech. 12:98-105 (1982)) or poly-D-(−)-3hydroxybutyric acid (EP 133,988).

Vectors of the Invention

Vectors comprising nucleic acids encoding the soluble NgR polypeptides may be used to produce soluble polypeptides for use in the methods of the invention. The choice of vector and expression control sequences to which such nucleic acids are operably linked depends on the functional properties desired, e.g., protein expression, and the host cell to be transformed.

In a typical embodiment, a soluble NgR polypeptide useful in the methods described herein is a recombinant protein produced by a cell (e.g., a CHO cell) that carries an exogenous nucleic acid encoding the protein. In other embodiments, the recombinant polypeptide is produced by a process commonly known as gene activation, wherein a cell that carries an exogenous nucleic acid that includes a promoter or enhancer is operably linked to an endogenous nucleic acid that encodes the polypeptide.

Routine techniques for making recombinant polypeptides (e.g., recombinant soluble NgR polypeptides) may be used to construct expression vectors encoding the polypeptides of interest using appropriate transcriptional/translational control signals and the protein coding sequences. (See, for example, Sambrook et al., Molecular Cloning: A Laboratory Manual, 3d Ed. (Cold Spring Harbor Laboratory 2001)). These methods may include in vitro recombinant DNA and synthetic techniques and in vivo recombination, e.g., in vivo homologous recombination. Expression of a nucleic acid sequence encoding a polypeptide may be regulated by a second nucleic acid sequence that is operably linked to the polypeptide encoding sequence such that the polypeptide is expressed in a host transformed with the recombinant DNA molecule.

Expression control elements useful for regulating the expression of an operably linked coding sequence are known in the art. Examples include, but are not limited to, inducible promoters, constitutive promoters, secretion signals, and other regulatory elements. When an inducible promoter is used, it can be controlled, e.g., by a change in nutrient status, or a change in temperature, in the host cell medium.

Expression vectors capable of being replicated in a bacterial or eukaryotic host comprising a nucleic acid encoding a polypeptide are used to transfect a host and thereby direct expression of such nucleic acid to produce the polypeptide, which may then be isolated. The preferred mammalian expression vectors contain both prokaryotic sequences, to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Routine techniques for transfecting cells with exogenous DNA sequences may be used in the present invention. Transfection methods may include chemical means, e.g., calcium phosphate, DEAE-dextran, or liposome; or physical means, e.g., microinjection or electroporation. The transfected cells are grown up by routine techniques. For examples, see Kuchler et al. (1977) Biochemical Methods in Cell Culture and Virology. The expression products are isolated from the cell medium in those systems where the protein is secreted from the host cell, or from the cell suspension after disruption of the host cell system by, e.g., routine mechanical, chemical, or enzymatic means. These methods may also be carried out using cells that have been genetically modified by other procedures, including gene targeting and gene activation (see Treco et al. WO 95/31560, herein incorporated by reference; see also Selden et al. WO 93/09222).

The vector can include a prokaryotic replicon, i.e., a DNA sequence having the ability to direct autonomous replication and maintenance of the recombinant DNA molecule extra-chromosomally in a bacterial host cell. Such replicons are well known in the art. In addition, vectors that include a prokaryotic replicon may also include a gene whose expression confers a detectable marker such as a drug resistance. Examples of bacterial drug-resistance genes are those that confer resistance to ampicillin or tetracycline.

Vectors that include a prokaryotic replicon can also include a prokaryotic or bacteriophage promoter for directing expression of the coding gene sequences in a bacterial host cell. Promoter sequences compatible with bacterial hosts are typically provided in plasmid vectors containing convenient restriction sites for insertion of a DNA segment to be expressed. Examples of such plasmid vectors are pUC8, pUC9, pBR322 and pBR329 (BioRad), pPL and pKK223 (Pharmacia). Any suitable prokaryotic host can be used to express a recombinant DNA molecule encoding a protein used in the methods of the invention.

For the purposes of this invention, numerous expression vector systems may be employed. For example, one class of vector utilizes DNA elements which are derived from animal viruses such as bovine papilloma virus, polyoma virus, adenovirus, adeno-associated virus, herpes simplex virus-1, vaccinia virus, baculovirus, retroviruses (RSV, MMTV or MOMLV) or SV40 virus. Examples of such vectors can be found in PCT publications WO 2006/060089 and WO2002/056918 which are incorporated herein in their entireties. Others involve the use of polycistronic systems with internal ribosome binding sites. Additionally, cells which have integrated the DNA into their chromosomes may be selected by introducing one or more markers which allow selection of transfected host cells. The marker may provide for prototrophy to an auxotrophic host, biocide resistance (e.g., antibiotics) or resistance to heavy metals such as copper. The selectable marker gene can either be directly linked to the DNA sequences to be expressed, or introduced into the same cell by cotransformation. The neomycin phosphotransferase (neo) gene is an example of a selectable marker gene (Southern et al., J. Mol. Anal. Genet. 1:327-341 (1982)). Additional elements may also be needed for optimal synthesis of mRNA. These elements may include signal sequences, splice signals, as well as transcriptional promoters, enhancers, and termination signals.

In one embodiment, a proprietary expression vector of Biogen IDEC, Inc., referred to as NEOSPLA (U.S. Pat. No. 6,159,730) may be used. This vector contains the cytomegalovirus promoter/enhancer, the mouse beta globin major promoter, the SV40 origin of replication, the bovine growth hormone polyadenylation sequence, neomycin phosphotransferase exon 1 and exon 2, the dihydrofolate reductase gene and leader sequence. This vector has been found to result in very high level expression upon transfection in CHO cells, followed by selection in G418 containing medium and methotrexate amplification. Of course, any expression vector which is capable of eliciting expression in eukaryotic cells may be used in the present invention. Examples of suitable vectors include, but are not limited to plasmids pcDNA3, pHCMV/Zeo, pCR3.1, pEF1/His, pIND/GS, pRc/HCMV2, pSV40/Zeo2, pTRACER-HCMV, pUB6/V5-His, pVAX1, and pZeoSV2 (available from Invitrogen, San Diego, Calif.), and plasmid pCI (available from Promega, Madison, Wis.). Additional eukaryotic cell expression vectors are known in the art and are commercially available. Typically, such vectors contain convenient restriction sites for insertion of the desired DNA segment. Exemplary vectors include pSVL and pKSV-10 (Pharmacia), pBPV-1, pml2d (International Biotechnologies), pTDT1 (ATCC 31255), retroviral expression vector pMIG and pLL3.7, adenovirus shuttle vector pDC315, and AAV vectors. Other exemplary vector systems are disclosed e.g., in U.S. Pat. No. 6,413,777.

In general, screening large numbers of transformed cells for those which express suitably high levels of the antagonist is routine experimentation which can be carried out, for example, by robotic systems.

The recombinant expression vectors may carry sequences that regulate replication of the vector in host cells (e.g., origins of replication) and selectable marker genes. It will be appreciated by those skilled in the art that the design of the expression vector, including the selection of regulatory sequences may depend on such factors as the choice of the host cell to be transformed, the level of expression of protein desired, etc. Frequently used regulatory sequences for mammalian host cell expression include viral elements that direct high levels of protein expression in mammalian cells, such as promoters and enhancers derived from retroviral LTRs, cytomegalovirus (CMV) (such as the CMV promoter/enhancer), Simian Virus 40 (SV40) (such as the SV40 promoter/enhancer), adenovirus, (e.g., the adenovirus major late promoter (Adm1P)), polyoma and strong mammalian promoters such as native immunoglobulin and actin promoters. For further description of viral regulatory elements, and sequences thereof, see e.g., Stinski, U.S. Pat. No. 5,168,062; Bell, U.S. Pat. No. 4,510,245; and Schaffner, U.S. Pat. No. 4,968,615.

The selectable marker gene facilitates selection of host cells into which the vector has been introduced (see, e.g., Axel, U.S. Pat. Nos. 4,399,216; 4,634,665 and 5,179,017). For example, typically the selectable marker gene confers resistance to a drug, such as G418, hygromycin or methotrexate, on a host cell into which the vector has been introduced. Frequently used selectable marker genes include the dihydrofolate reductase (DHFR) gene (for use in dhfr-host cells with methotrexate selection/amplification) and the neo gene (for G418 selection).

Vectors comprising polynucleotides encoding soluble NgR polypeptides can be used for transformation of a suitable host cell. Transformation can be by any suitable method. Methods for introduction of exogenous DNA into mammalian cells are well known in the art and include dextran-mediated transfection, calcium phosphate precipitation, polybrene-mediated transfection, protoplast fusion, electroporation, encapsulation of the polynucleotide(s) in liposomes, and direct microinjection of the DNA into nuclei. In addition, nucleic acid molecules may be introduced into mammalian cells by viral vectors.

Transformation of host cells can be accomplished by conventional methods suited to the vector and host cell employed. For transformation of prokaryotic host cells, electroporation and salt treatment methods can be employed (Cohen et al., Proc. Natl. Acad. Sci. USA 69:2110-14 (1972)). For transformation of vertebrate cells, electroporation, cationic lipid or salt treatment methods can be employed. See, e.g., Graham et al., Virology 52:456-467 (1973); Wigler et al., Proc. Natl. Acad. Sci. USA 76:1373-76 (1979).

The host cell line used for protein expression is most preferably of mammalian origin; those skilled in the art are credited with ability to preferentially determine particular host cell lines which are best suited for the desired gene product to be expressed therein. Exemplary host cell lines include, but are not limited to NSO, SP2 cells, baby hamster kidney (BHK) cells, monkey kidney cells (COS), human hepatocellular carcinoma cells (e.g., Hep G2), A549 cells DG44 and DUXB11 (Chinese Hamster Ovary lines, DHFR minus), HELA (human cervical carcinoma), CV1 (monkey kidney line), COS (a derivative of CV1 with SV40 T antigen), R1610 (Chinese hamster fibroblast) BALBC/3T3 (mouse fibroblast), HAK (hamster kidney line), SP2/O (mouse myeloma), P3×63-Ag3.653 (mouse myeloma), BFA-1c1BPT (bovine endothelial cells), RAJI (human lymphocyte) and 293 (human kidney). Host cell lines are typically available from commercial services, the American Tissue Culture Collection or from published literature.

Expression of polypeptides from production cell lines can be enhanced using known techniques. For example, the glutamine synthetase (GS) system is commonly used for enhancing expression under certain conditions. See, e.g., European Patent Nos. 0 216 846, 0 256 055, and 0 323 997 and European Patent Application No. 89303964.4.

In some embodiments, the invention provides recombinant DNA molecules (rDNA) that contain a coding sequence. As used herein, a rDNA molecule is a DNA molecule that has been subjected to molecular manipulation. Methods for generating rDNA molecules are well known in the art, for example, see Sambrook et al., Molecular Cloning—A Laboratory Manual, Cold Spring Harbor Laboratory Press (1989). In some rDNA molecules, a coding DNA sequence is operably linked to expression control sequences and vector sequences. A vector of the present invention may be at least capable of directing the replication or insertion into the host chromosome, and preferably also expression, of the structural gene included in the rDNA molecule.

Expression vectors compatible with eukaryotic cells, preferably those compatible with vertebrate cells, can also be used to form a rDNA molecules that contains a coding sequence. Eukaryotic cell expression vectors are well known in the art and are available from several commercial sources. Typically, such vectors are provided containing convenient restriction sites for insertion of the desired DNA segment. Examples of such vectors are pSVL and pKSV-10 (Pharmacia), pBPV-1, pML2d (International Biotechnologies), pTDT1 (ATCC® 31255) and other eukaryotic expression vectors.

Eukaryotic cell expression vectors used to construct the rDNA molecules of the present invention may further include a selectable marker that is effective in an eukaryotic cell, preferably a drug resistance selection marker. A preferred drug resistance marker is the gene whose expression results in neomycin resistance, i.e., the neomycin phosphotransferase (neo) gene. (Southern et al., J. Mol. Anal. Genet. 1:327-341 (1982)). Alternatively, the selectable marker can be present on a separate plasmid, the two vectors introduced by co-transfection of the host cell, and transfectants selected by culturing in the appropriate drug for the selectable marker.

To express the antibodies, or antibody portions of the invention, DNAs encoding partial or full-length light and heavy chains are inserted into expression vectors such that the genes are operatively linked to transcriptional and translational control sequences. Expression vectors include plasmids, retroviruses, cosmids, YACs, EBV-derived episomes, and the like. The antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vectors. In some embodiments, both genes are inserted into the same expression vector. The antibody genes are inserted into the expression vector by standard methods (e.g., ligation of complementary restriction sites on the antibody gene fragment and vector, or blunt end ligation if no restriction sites are present).

A convenient vector is one that encodes a functionally complete human C_{H} or C_{L} immunoglobulin sequence, with appropriate restriction sites engineered so that any V_{H} or V_{L} sequence can be easily inserted and expressed, as described above. In such vectors, splicing usually occurs between the splice donor site in the inserted J region and the splice acceptor site preceding the human C region, and also at the splice regions that occur within the human C_{H} exons. Polyadenylation and transcription termination occur at native chromosomal sites downstream of the coding regions. The recombinant expression vector can also encode a signal peptide that facilitates secretion of the antibody chain from a host cell. The antibody chain gene may be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

Other embodiments of the invention use a lentiviral vector for expression of the polynucleotides of the invention. Lentiviruses can infect noncycling and postmitotic cells, and also provide the advantage of not being silenced during development allowing generation of transgenic animals through infection of embryonic stem cells. Milhavet et al., Pharmacological Rev. 55:629-648 (2003). Other polynucleotide expressing viral vectors can be constructed based on, but not limited to, adeno-associated virus, retrovirus, adenovirus, or alphavirus.

Transcription of the polynucleotides of the invention can be driven from a promoter for eukaryotic RNA polymerase I (pol I), RNA polymerase II (pol II), or RNA polymerase Ill (pol III). Transcripts from pol II or pol III promoters are expressed at high levels in all cells; the levels of a given pol II promoter in a given cell type depends on the nature of the gene regulatory sequences (enhancers, silencers, etc.) present nearby. Prokaryotic RNA polymerase promoters are also used, providing that the prokaryotic RNA polymerase enzyme is expressed in the appropriate cells (Elroy-Stein and Moss, Proc. Natl. Acad. Sci. USA 87:6743-7 (1990); Gao and Huang, Nucleic Acids Res. 21:2867-72 (1993); Lieber et al., Methods Enzymol. 217:47-66 (1993); Zhou et al., Mol. Cell. Biol. 10:4529-37 (1990)). Several investigators have demonstrated that polynucleotides expressed from such promoters can function in mammalian cells (e.g. Kashani-Sabet et al., Antisense Res. Dev. 2:3-15 (1992); Ojwang et al., Proc. Natl. Acad. Sci. USA 89:10802-6 (1992); Chen et al., Nucleic Acids Res. 20:4581-9 (1992); Yu et al., Proc. Natl. Acad. Sci. USA 90:6340-4 (1993); L′Huillier et al., EMBO J. 11:4411-8 (1992); Lisziewicz et al., Proc. Natl. Acad. Sci. U.S.A 90:8000-4 (1993); Thompson et al., Nucleic Acids Res. 23:2259 (1995); Sullenger & Cech, Science 262:1566 (1993)).

Host Cells and Methods of Recombinantly Producing Protein of the Invention

Nucleic acid molecules encoding soluble Nogo receptor polypeptides, soluble Nogo receptor fusion proteins of this invention and vectors comprising these nucleic acid molecules can be used for transformation of a suitable host cell. Transformation can be by any known method for introducing polynucleotides into a host cell. Methods for introduction of heterologous polynucleotides into mammalian cells are well known in the art and include dextran-mediated transfection, calcium phosphate precipitation, polybrene-mediated transfection, protoplast fusion, electroporation, encapsulation of the polynucleotide(s) in liposomes, and direct microinjection of the DNA into nuclei. In addition, nucleic acid molecules may be introduced into mammalian cells by viral vectors.

Transformation of appropriate cell hosts with a rDNA molecule of the present invention is accomplished by well known methods that typically depend on the type of vector used and host system employed. With regard to transformation of prokaryotic host cells, electroporation and salt treatment methods can be employed (see, for example, Sambrook et al., Molecular Cloning—A Laboratory Manual, Cold Spring Harbor Laboratory Press (1989); Cohen et al., Proc. Natl. Acad. Sci. USA 69:2110-2114 (1972)). With regard to transformation of vertebrate cells with vectors containing rDNA, electroporation, cationic lipid or salt treatment methods can be employed (see, for example, Graham et al., Virology 52:456-467 (1973); Wigler et al., Proc. Natl. Acad. Sci. USA 76:1373-1376 (1979)).

Successfully transformed cells, i.e., cells that contain a rDNA molecule of the present invention, can be identified by well known techniques including the selection for a selectable marker. For example, cells resulting from the introduction of an rDNA of the present invention can be cloned to produce single colonies. Cells from those colonies can be harvested, lysed and their DNA content examined for the presence of the rDNA using a method such as that described by Southern, J. Mol. Biol. 98:503-517 (1975) or the proteins produced from the cell may be assayed by an immunological method.

Host cells for expression of a polypeptide or antibody of the invention for use in a method of the invention may be prokaryotic or eukaryotic. Mammalian cell lines available as hosts for expression are well known in the art and include many immortalized cell lines available from the American Type Culture Collection (ATCC®). These include, inter alfa, Chinese hamster ovary (CHO) cells, NSO, SP2 cells, HeLa cells, baby hamster kidney (BHK) cells, monkey kidney cells (COS), human hepatocellular carcinoma cells (e.g., Hep G2), A549 cells, and a number of other cell lines. Cell lines of particular preference are selected through determining which cell lines have high expression levels. Other useful eukaryotic host cells include plant cells. Other cell lines that may be used are insect cell lines, such as Sf9 cells. Exemplary prokaryotic host cells are E. coli and Streptomyces.

When recombinant expression vectors encoding the soluble Nogo receptor polypeptides and soluble Nogo receptor fusion proteins of the invention are introduced into mammalian host cells, they are produced by culturing the host cells for a period of time sufficient to allow for expression of the antibody, polypeptide and fusion polypeptide in the host cells or, more preferably, secretion of the soluble Nogo receptor polypeptides and soluble Nogo receptor fusion proteins of the invention into the culture medium in which the host cells are grown. Soluble Nogo receptor polypeptides and soluble Nogo receptor fusion proteins of the invention can be recovered from the culture medium using standard protein purification methods.

Further, expression of soluble Nogo receptor polypeptides and soluble Nogo receptor fusion proteins of the invention of the invention (or other moieties therefrom) from production cell lines can be enhanced using a number of known techniques. For example, the glutamine synthetase gene expression system (the GS system) is a common approach for enhancing expression under certain conditions. The GS system is discussed in whole or part in connection with European Patent Nos. 0 216 846, 0 256 055, and 0 323 997 and European Patent Application No. 89303964.4.

A polypeptide produced by a cultured cell as described herein can be recovered from the culture medium as a secreted polypeptide, or, if it is not secreted by the cells, it can be recovered from host cell lysates. As a first step in isolating the polypeptide, the culture medium or lysate is generally centrifuged to remove particulate cell debris. The polypeptide thereafter is isolated, and preferably purified, from contaminating soluble proteins and other cellular components, with the following procedures being exemplary of suitable purification procedures: fractionation on immunoaffinity or ion-exchange columns; ethanol precipitation; reverse phase HPLC; chromatography on silica or on a cation-exchange resin such as DEAE; chromatofocusing; SDS PAGE; ammonium sulfate precipitation; and gel filtration, e.g., with Sephadex™ columns (Amersham Biosciences). Protease inhibitors may be used to inhibit proteolytic degradation during purification. One skilled in the art will appreciate that purification methods suitable for the polypeptide of interest may require modification to account for changes in the character of the polypeptide upon expression in recombinant cell culture.

The purification of polypeptides may require the use of, e.g., affinity chromatography, conventional ion exchange chromatography, sizing chromatography, hydrophobic interaction chromatography, reverse phase chromatography, gel filtration or other conventional protein purification techniques. See, e.g., Deutscher, ed. (1990) “Guide to Protein Purification” in Methods in Enzymology, Vol. 182.

### Cell Therapy

In some embodiments of the invention a soluble NgR polypeptide is administered in a treatment method that includes: (1) transforming or transfecting an implantable host cell with a nucleic acid, e.g., a vector, that expresses a soluble NgR polypeptide; and (2) implanting the transformed host cell into a mammal, at the site of a disease, disorder or injury. For example, the transformed host cell can be implanted at the site of a spinal cord injury. In some embodiments of the invention, the implantable host cell is removed from a mammal, temporarily cultured, transformed or transfected with an isolated nucleic acid encoding a soluble NgR polypeptide, and implanted back into the same mammal from which it was removed. The cell can be, but is not required to be, removed from the same site at which it is implanted. Such embodiments, sometimes known as ex vivo gene therapy, can provide a continuous supply of the soluble NgR polypeptide, localized at the site of site of action, for a limited period of time.

### Gene Therapy

A soluble NgR polypeptide can be produced in vivo in a mammal, e.g., a human patient, using a gene-therapy approach to treatment of a disease, disorder or injury in which reducing Aβ accumulation would be therapeutically beneficial. This involves administration of a suitable soluble NgR polypeptide-encoding nucleic acid operably linked to suitable expression control sequences. Generally, these sequences are incorporated into a viral vector. Suitable viral vectors for such gene therapy include an adenoviral vector, an alphavirus vector, an enterovirus vector, a pestivirus vector, a lentiviral vector, a baculoviral vector, a herpesvirus vector, an Epstein Barr viral vector, a papovaviral vector, a poxvirus vector, a vaccinia viral vector, adeno-associated viral vector and a herpes simplex viral vector. The viral vector can be a replication-defective viral vector. Adenoviral vectors that have a deletion in its E1 gene or E3 gene are typically used. When an adenoviral vector is used, the vector usually does not have a selectable marker gene. Examples of such vectors can be found in PCT publications WO 2006/060089 and WO2002/056918 which are incorporated herein in their entireties. Expression constructs of a soluble NgR polypeptide may be administered in any biologically effective carrier, e.g. any formulation or composition capable of effectively delivering the soluble NgR polypeptide gene to cells in vivo. Approaches include insertion of the subject gene in viral vectors including recombinant retroviruses, adenovirus, adeno-associated virus, and herpes simplex virus-1, or recombinant bacterial or eukaryotic plasmids. Viral vectors transfect cells directly; plasmid DNA can be delivered with the help of, for example, cationic liposomes (lipofectin) or derivatized (e.g. antibody conjugated), polylysine conjugates, gramacidin S, artificial viral envelopes or other such intracellular carriers, as well as direct injection of the gene construct or CaPO₄ precipitation carried out in vivo.

A preferred approach for in vivo introduction of nucleic acid into a cell is by use of a viral vector containing nucleic acid, e.g. a cDNA, encoding a soluble NgR polypeptide, or a soluble NgR polypeptide antisense nucleic acid. Infection of cells with a viral vector has the advantage that a large proportion of the targeted cells can receive the nucleic acid. Additionally, molecules encoded within the viral vector, e.g., by a cDNA contained in the viral vector, are expressed efficiently in cells which have taken up viral vector nucleic acid.

Retrovirus vectors and adeno-associated virus vectors can be used as a recombinant gene delivery system for the transfer of exogenous genes in vivo, particularly into humans. These vectors provide efficient delivery of genes into cells, and the transferred nucleic acids are stably integrated into the chromosomal DNA of the host. The development of specialized cell lines (termed “packaging cells”) which produce only replication-defective retroviruses has increased the utility of retroviruses for gene therapy, and defective retroviruses are characterized for use in gene transfer for gene therapy purposes. A replication defective retrovirus can be packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are known to those skilled in the art. Examples of suitable packaging virus lines for preparing both ecotropic and amphotropic retroviral systems include .psi.Crip, .psi.Cre, .psi.2 and .psi.Am. Retroviruses have been used to introduce a variety of genes into many different cell types, including epithelial cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573).

Another viral gene delivery system useful in the present invention utilizes adenovirus-derived vectors. The genome of an adenovirus can be manipulated such that it encodes and expresses a gene product of interest but is inactivated in terms of its ability to replicate in a normal lytic viral life cycle. See, for example, Berkner et al. (1988) BioTechniques 6:616; Rosenfeld et al. (1991) Science 252:431-434; and Rosenfeld et al. (1992) Cell 68:143-155. Suitable adenoviral vectors derived from the adenovirus strain Ad type 5 d1324 or other strains of adenovirus (e.g., Ad2, Ad3, Ad7 etc.) are known to those skilled in the art. Recombinant adenoviruses can be advantageous in certain circumstances in that they are not capable of infecting nondividing cells and can be used to infect a wide variety of cell types, including epithelial cells (Rosenfeld et al. (1992) cited supra). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situ where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al. cited supra; Haj-Ahmand and Graham (1986) J. Virol. 57:267).

Yet another viral vector system useful for delivery of the subject gene is the adeno-associated virus (AAV). Reviewed in Ali, 2004, Novartis Found Symp. 255:165-78; and Lu, 2004, Stem Cells Dev. 13(1):133-45. Adeno-associated virus is a naturally occurring defective virus that requires another virus, such as an adenovirus or a herpes virus, as a helper virus for efficient replication and a productive life cycle. (For a review see Muzyczka et al. (1992) Curr. Topics in Micro. and Immunol. 158:97-129). It is also one of the few viruses that may integrate its DNA into non-dividing cells, and exhibits a high frequency of stable integration (see for example Flotte et al. (1992) Am. J. Respir. Cell. Mol. Biol. 7:349-356; Samulski et al. (1989) J. Virol. 63:3822-3828; and McLaughlin et al. (1989) J. Virol. 62:1963-1973). Vectors containing as little as 300 base pairs of AAV can be packaged and can integrate. Space for exogenous DNA is limited to about 4.5 kb. An AAV vector such as that described in Tratschin et al. (1985) Mol. Cell. Biol. 5:3251-3260 can be used to introduce DNA into cells. A variety of nucleic acids have been introduced into different cell types using AAV vectors (see for example Hennonat et al. (1984) Proc. Natl. Acad. Sci. USA 81:6466-6470; Tratschin et al. (1985) Mol. Cell. Biol. 4:2072-2081; Wondisford et al. (1988) Mol. Endocrinol. 2:32-39; Tratschin et al. (1984) J. Virol. 51:611-619; and Flotte et al. (1993) J. Biol. Chem. 268:3781-3790).

In addition to viral transfer methods, such as those illustrated above, non-viral methods can also be employed to cause expression of a soluble NgR polypeptide, fragment, or analog, in the tissue of an animal. Most nonviral methods of gene transfer rely on normal mechanisms used by mammalian cells for the uptake and intracellular transport of macromolecules. In preferred embodiments, non-viral gene delivery systems of the present invention rely on endocytic pathways for the uptake of the subject NgR gene by the targeted cell. Exemplary gene delivery systems of this type include liposomal derived systems, poly-lysine conjugates, and artificial viral envelopes. Other embodiments include plasmid injection systems such as are described in Meuli et al. (2001) J Invest Dermatol. 116(1):131-135; Cohen et al. (2000) Gene Ther 7(22):1896-905; or Tam et al. (2000) Gene Ther 7(21):1867-74.

In a representative embodiment, a gene encoding a soluble NgR polypeptide, active fragment, or analog, can be entrapped in liposomes bearing positive charges on their surface (e.g., lipofectins) and (optionally) which are tagged with antibodies against cell surface antigens of the target tissue (Mizuno et al. (1992) No Shinkei Geka 20:547-551; PCT publication WO91/06309; Japanese patent application 1047381; and European patent publication EP-A-43075).

In clinical settings, the gene delivery systems for the therapeutic NgR gene can be introduced into a patient by any of a number of methods, each of which is familiar in the art. For instance, a pharmaceutical preparation of the gene delivery system can be introduced systemically, e.g. by intravenous injection, and specific transduction of the protein in the target cells occurs predominantly from specificity of transfection provided by the gene delivery vehicle, cell-type or tissue-type expression due to the transcriptional regulatory sequences controlling expression of the receptor gene, or a combination thereof. In other embodiments, initial delivery of the recombinant gene is more limited with introduction into the animal being quite localized. For example, the gene delivery vehicle can be introduced by catheter (see U.S. Pat. No. 5,328,470) or by stereotactic injection (e.g. Chen et al. (1994) Pros. Natl. Acad. Sci. USA 91: 3054-3057).

The pharmaceutical preparation of the gene therapy construct can consist essentially of the gene delivery system in an acceptable diluent, or can comprise a slow release matrix in which the gene delivery vehicle is imbedded. Alternatively, where the complete gene delivery system can be produced intact from recombinant cells, e.g. retroviral vectors, the pharmaceutical preparation can comprise one or more cells which produce the gene delivery system.

Guidance regarding gene therapy in particular for treating a CNS condition or disorder as described herein can be found, e.g., in U.S. patent application Ser. No. 2002/0,193,335 (provides methods of delivering a gene therapy vector, or transformed cell, to neurological tissue); U.S. patent application Ser. No. 2002/0,187,951 (provides methods for treating a neurodegenerative disease using a lentiviral vector to a target cell in the brain or nervous system of a mammal); U.S. patent application Ser. No. 2002/0,107,213 (discloses a gene therapy vehicle and methods for its use in the treatment and prevention of neurodegenerative disease); U.S. patent application Ser. No. 2003/0,099,671 (discloses a mutated rabies virus suitable for delivering a gene to the CNS); and U.S. Pat. No. 6,436,708 (discloses a gene delivery system which results in long-term expression throughout the brain); U.S. Pat. No. 6,140,111 (discloses retroviral vectors suitable for human gene therapy in, the treatment of a variety of disease); and Kaspar et al. (2002) Mol. Ther. 5:50-6; Sulu et al (1999) Arch Neurol. 56:287-92; and Wong et al. (2002) Nat Neurosci 5, 633-639).

Production of Recombinant Proteins using a rDNA Molecule

The present invention further provides methods for producing a soluble Nogo receptor polypeptide and/or soluble Nogo receptor fusion protein of the invention using nucleic acid molecules herein described. In general terms, the production of a recombinant form of a protein typically involves the following steps: First, a nucleic acid molecule is obtained that encodes a protein of the invention. If the encoding sequence is uninterrupted by introns, it is directly suitable for expression in any host. The nucleic acid molecule is then optionally placed in operable linkage with suitable control sequences, as described above, to form an expression unit containing the protein open reading frame. The expression unit is used to transform a suitable host and the transformed host is cultured under conditions that allow the production of the recombinant protein. Optionally the recombinant protein is isolated from the medium or from the cells; recovery and purification of the protein may not be necessary in some instances where some impurities may be tolerated.

Each of the foregoing steps can be done in a variety of ways. For example, the desired coding sequences may be obtained from genomic fragments and used directly in appropriate hosts. The construction of expression vectors that are operable in a variety of hosts is accomplished using appropriate replicons and control sequences, as set forth above. The control sequences, expression vectors, and transformation methods are dependent on the type of host cell used to express the gene and were discussed in detail earlier. Suitable restriction sites can, if not normally available, be added to the ends of the coding sequence so as to provide an excisable gene to insert into these vectors. A skilled artisan can readily adapt any host/expression system known in the art for use with the nucleic acid molecules of the invention to produce recombinant protein.

Methods Using Soluble NgR polypeptides, Fusion Proteins, Polynucleotides and Compositions

One embodiment of the present invention provides a method for increasing the plasma to brain ratio of Aβ peptide in a mammal, comprising administering a therapeutically effective amount of a soluble Nogo receptor polypeptide peripheral to the central nervous system.

Another embodiment of the invention provides a method for enhancing Aβ clearance from the brain of a mammal, comprising administering a therapeutically effective amount of a soluble Nogo receptor polypeptide peripheral to the central nervous system.

A further embodiment of the invention provides a method for improving memory function or inhibiting memory loss in a mammal comprising administering a therapeutically effective amount of a soluble Nogo receptor polypeptide peripheral to the central nervous system.

Another embodiment of the invention provides a method of reducing the number of Aβ plaques in the brain of a mammal, comprising administering to a mammal in need thereof a therapeutically effective amount of a soluble Nogo receptor polypeptide, wherein said administration is peripheral to the central nervous system.

Another embodiment of the invention provides a method of reducing the size of Aβ plaques in the brain of a mammal, comprising administering to a mammal in need thereof a therapeutically effective amount of a soluble Nogo receptor polypeptide, wherein said administration is peripheral to the central nervous system.

Another embodiment of the invention provides a method of treating a disease associated with Aβ plaque accumulation in a mammal comprising administering to a mammal in need thereof a therapeutically effective amount of a soluble Nogo receptor polypeptide, wherein said administration is peripheral to the central nervous system.

Disease that can be treated using the methods of the present invention include but are not limited to Alzheimer's disease, mild cognitive impairment, mild-to-moderate cognitive impairment, vascular dementia, cerebral amyloid angiopathy, hereditary cerebral hemorrhage, senile dementia, Down's syndrome, inclusion body myositis, age-related macular degeneration, primary amyloidosis, secondary amyloidosis or a condition associated with Alzheimer's disease. Conditions associated with Alzheimer's disease that can be treated using the methods of the present invention include but are not limited to hypothyroidism, cerebrovascular disease, cardiovascular disease, memory loss, anxiety, a behavioral dysfunction, a neurological condition, or a psychological condition. Behavioral dysfunction that can be treated using the methods of the present invention include but is not limited to apathy, aggression, or incontinence. Neurological conditions that can be treated using the methods of the present invention include but are not limited to Huntington's disease, amyotrophic lateral sclerosis, acquired immunodeficiency, Parkinson's disease, aphasia, apraxia, agnosia, Pick disease, dementia with Lewy bodies, altered muscle tone, seizures, sensory loss, visual field deficits, incoordination, gait disturbance, transient ischemic attack or stroke, transient alertness, attention deficit, frequent falls, syncope, neuroleptic sensitivity, normal pressure hydrocephalus, subdural hematoma, brain tumor, posttraumatic brain injury, or posthypoxic damage. Psychological conditions that can be treated using the methods of the present invention include but are not limited to depression, delusions, illusions, hallucinations, sexual disorders, weight loss, psychosis, a sleep disturbance, insomnia, behavioral disinhibition, poor insight, suicidal ideation, depressed mood, irritability, anhedonia, social withdrawal, or excessive guilt.

Mild cognitive impairment (MCI) is a condition characterized by a state of mild but measurable impairment in thinking skills, but is not necessarily associated with the presence of dementia. MCI frequently, but not necessarily, precedes Alzheimer's disease. It is a diagnosis that has most often been associated with mild memory problems, but it can also be characterized by mild impairments in other thinking skills, such as language or planning skills. However, in general, an individual with MCI will have more significant memory lapses than would be expected for someone of their age or educational background. As the condition progresses, a physician may change the diagnosis to Mild-to-Moderate Cognition Impairment, as is well understood in this art.

In methods of the present invention, a soluble NgR polypeptide is administered peripheral to the central nervous system. “Peripheral to the central nervous system” includes any route of administration except for those routes of administration wherein the NgR polypeptide is administered directly to the central nervous system, e.g., intracerebroventricularly, or intrathecally. The soluble Nogo receptor polypeptides or Nogo receptor fusion proteins of the present invention can be administered via parenteral, subcutaneous, intravenous, intramuscular, intraperitoneal, transdermal, inhalational or buccal routes. For example, an agent may be administered locally to a site of injury via microinfusion. In one embodiment, the soluble NgR polypeptide is administered subcutaneously. In some embodiments of the present invention, the soluble NgR polypeptide does not cross the blood-brain barrier (BBB).

The soluble Nogo receptor polypeptides or fusion proteins of the present invention can be provided alone, or in combination, or in sequential combination with other agents that modulate a particular pathological process. As used herein, the soluble Nogo receptor and Nogo receptor fusion proteins, are said to be administered in combination with one or more additional therapeutic agents when the two are administered simultaneously, consecutively or independently.

In some embodiments, an NgR receptor polypeptide or fusion protein may be coformulated with and/or coadministered with one or more anti-Aβ antibodies for use in the methods of the present invention. Examples of anti-AR for use in the methods of the present invention can be found, e.g., in U.S Patent Publication Nos. 20060165682 A1, 20060039906 A1, and 20040043418 A1.

In some embodiments, an NgR1 polypeptide or fusion protein may be coformulated with and/or coadministered with one or more additional therapeutic agents, such as an adrenergic agent, anti-adrenergic agent, anti-androgen agent, anti-anginal agent, anti-anxiety agent, anticonvulsant agent, antidepressant agent, anti-epileptic agent, antihyperlipidemic agent, antihyperlipoproteinemic agent, antihypertensive agent, anti-inflammatory agent, antiobessional agent, antiparkinsonian agent, antipsychotic agent, adrenocortical steroid; adrenocortical suppressant; aldosterone antagonist; amino acid; anabolic steroid; analeptic agent; androgen; blood glucose regulator; cardioprotectant agent; cardiovascular agent; cholinergic agonist or antagonist; cholinesterase deactivator or inhibitor, cognition adjuvant or enhancer; dopaminergic agent; enzyme inhibitor, estrogen, free oxygen radical scavenger; GABA agonist; glutamate antagonist; hormone; hypocholesterolemic agent; hypolipidemic agent; hypotensive agent; immunizing agent; immunostimulant agent; monoamine oxidase inhibitor, neuroprotective agent; NMDA antagonist; AMPA antagonist, competitive or-non-competitive NMDA antagonist; opioid antagonist; potassium channel opener; non-hormonal sterol derivative; post-stroke and post-head trauma treatment; prostaglandin agent; psychotropic agent; relaxant agent; sedative agent; sedative-hypnotic agent; selective adenosine antagonist; serotonin antagonist; serotonin inhibitor; selective serotonin uptake inhibitor; serotonin receptor antagonist; sodium and calcium channel blocker; steroid; stimulant; and thyroid hormone and inhibitor agents for use in the methods of the present invention.

The dosage administered will be dependent upon the age, health, and weight of the recipient, kind of concurrent treatment, if any, frequency of treatment, and the nature of the effect desired. The compounds of this invention can be utilized in vivo, ordinarily in mammals, such as humans, sheep, horses, cattle, pigs, dogs, cats, rats and mice, or in vitro.

The methods of treatment of diseases and disorders as described herein are typically tested in vitro, and then in vivo in an acceptable animal model, for the desired therapeutic or prophylactic activity, prior to use in humans. Suitable animal models, including transgenic animals, are will known to those of ordinary skill in the art. The effect of the NgR1 polypeptides, fusion proteins, and compositions on increasing the brain to plasma ratio of Aβ peptide and enhancing Aβ clearance from the brain and reducing the number of Aβ plaques can be tested in vitro as described in the Examples. Finally, in vivo tests can be performed by creating transgenic mice which express the appropriate phenotype and administering the NgR1 polypeptides to mice or rats in models as described herein.

It will be readily apparent to one of ordinary skill in the relevant arts that other suitable modifications and adaptations to the methods and applications described herein are obvious and may be made without departing from the scope of the invention or any embodiment thereof. In order that this invention may be better understood, the following examples are set forth. These examples are for purposes of illustration only and are not to be construed as limiting the scope of the invention in any manner.

### Example 1

### Residues 15-28 in Aβ(1-28) are Essential for Binding to NgR

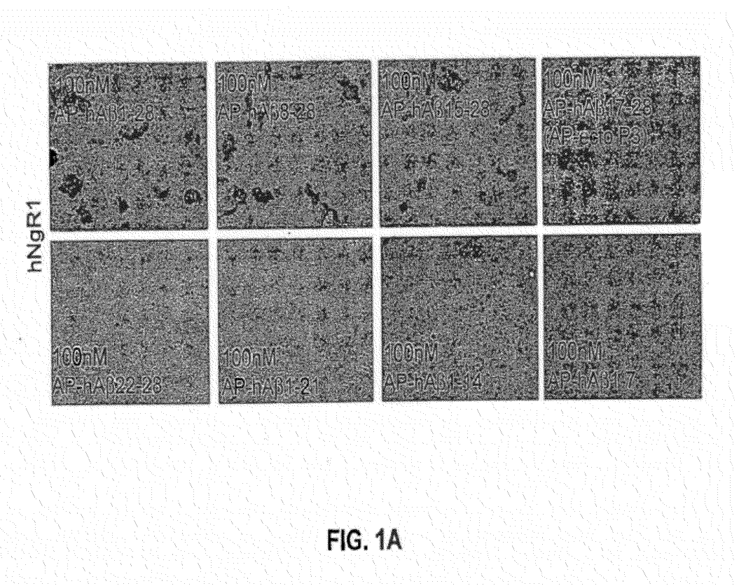

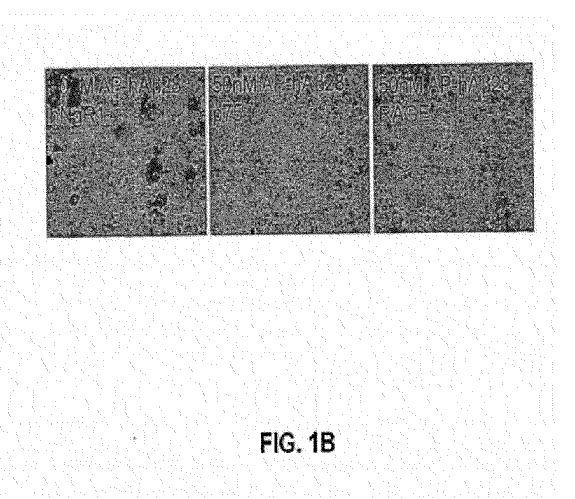

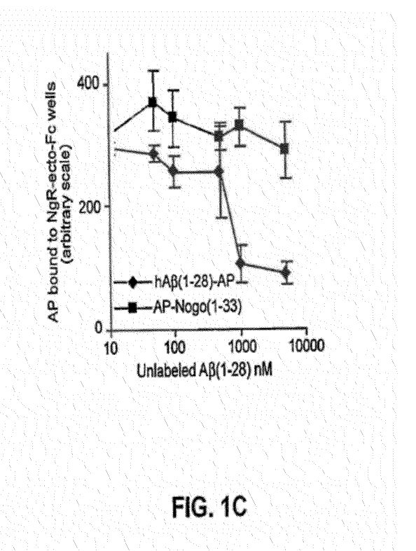

To determine whether a linear subsegment of Aβ(1-28) might interact with full-length human NgR in a cell-binding assay, deletion constructs containing various portions of the Aβ ectodomain fused to AP were created. AP-Aβ(1-28) protein was produced by the same method as AP-Nogo-66. To generate AP-Aβ mutant constructs, Aβ fragments were amplified, ligated into the pAP5tag vector (GenHunter) and sequenced. Recombinant proteins were confirmed by immunoblotting. The binding of AP fusion proteins to transfected COS-7 cells has been described previously. Fournier et al., Nature 409:341-346 (2001). The region of Aβ responsible for full-length human NgR interaction localizes to residues 15-28, the central residues of Aβ 1-40 (FIG. 1a).

The binding of AP-Aβ(1-28) to NgR with that to other reported partners, p75 and RAGE was also compared. Deane et al., Nat Med 9:907-913 (2003); Yaar et al., J Clin Invest 100:2333-2340 (1997). COS-7 cells were transfected with p75-N′ fR and RAGE, membrane proteins reported to bind Aβ. Deane et al., Nat Med 9:907-913 (2003); Yaar et al., J Clin Invest 100:2333-2340 (1997). Under conditions where AP-Aβ(1-28) binding to NgR is readily detectable, p75 and RAGE do not exhibit significant interaction with AB fusion protein (FIG. 1b).

NgR was identified by virtue of its affinity for Nogo-66, so we considered whether Aβ and Nogo-66 compete for binding to NgR. Competition was assessed in binding assays of AP-Aβ(1-28) or AP-Nogo66(1-33) to immobilized, purified NgR protein. Synthetic Aβ□1-28 was used to assess AP-Aβ(1-28) and AP-Nogo-66(1-33) displacement from immobilized human NgR(310)ecto-Fc in an ELISA format. 250 nM soluble AP-Aβ(1-28) or AP-Nogo-66(1-33) was allowed to bind to wells coated with purified NgR(310)ecto-Fc in the presence of the indicated concentrations of free Aβ□(1-28). In this cell-free assay, avidity for NgR is reduced compare to the cell based binding system, and the measured Kᵢ for Aβ(131-28) is 700 nM. Data are means +/−SEM from 4 experiments. Synthetic Aβ(1-28) disrupts NgR's ability to interact with the Aβ ligand but not the Nogo-66 ligand (FIG. 1c). Thus, the two ligand binding sites of NgR are distinguished by this assay.

### Example 2

### Specific Residues in NgR Support Binding to Ap-Aβ(1-28)

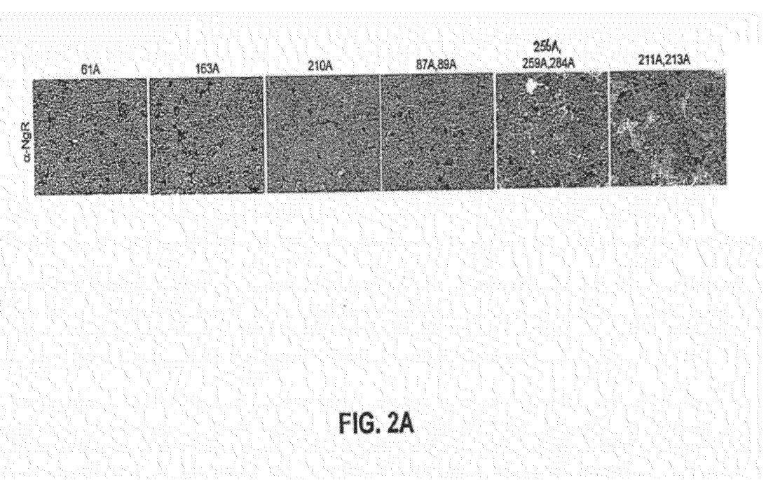

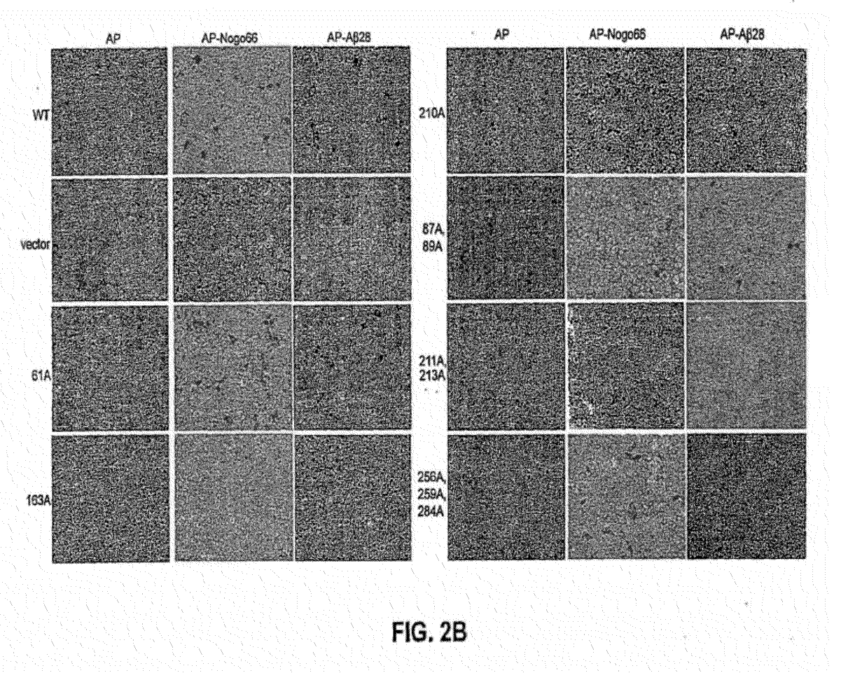

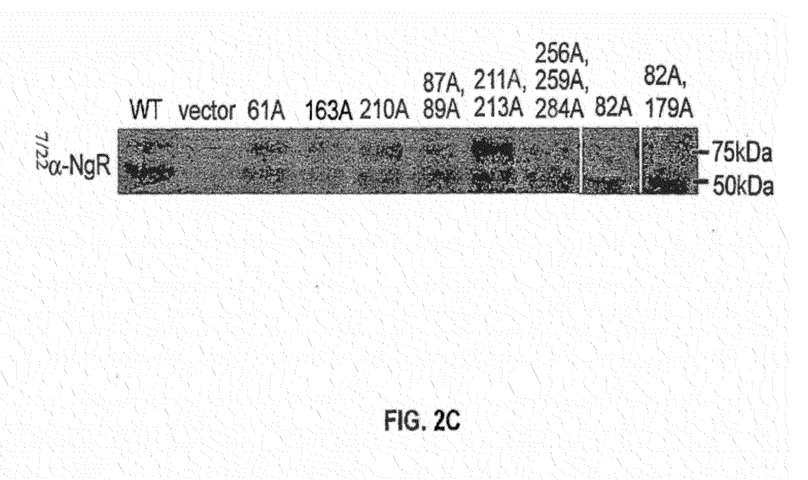

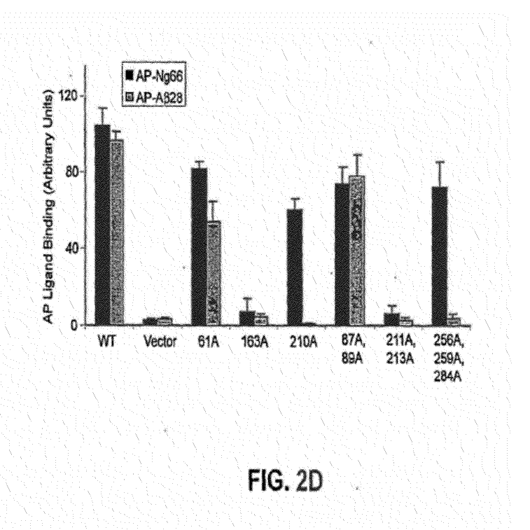

In order to probe the NgR domains that interact with Aβ and Nogo-66, a strategy based on the crystal structure of NgR was employed. A number of human NgR surface-accessible residues were mutated to Ala either individually or as groups of adjacent residues (Table 3), and resultant ligand binding characteristics were assessed. The ligand concentrations were AP, 30 nM, AP-Nogo-66, 5 nM, AP-Aβ(1-28), 50 nM. NgR mutagenesis has been previously described. Hu et al., J Neurosci 25:5298-5304 (2005); Fournier et al., J Neurosci 23:1416-1423 (2003). The expression of each mutant NgR protein was verified by immunohistochemical detection at the surface of cells transfected with expression vector (FIG. 2a). Bound AP was stained and measured using NIH image software. Mutants of human NgR were detected on the surface of transfected COS-7 immunofluorescently. For each of the mutants with altered binding characteristics, expression of immunoreactive NgR protein with electrophoretic mobility similar to wild type was also confirmed by immunoblot (FIG. 2c). Whole COS-7 cell lysates expressing NgR mutants were subjected to SDS-PAGE and blotted with anti-NgR antibodies. The mobility of each mutant was indistinguishable from wild type NgR, except for the mutations in N-linked glycosylation sites (N82 and N179). The binding of both AP-Aβ(1-28) and AP-Nogo-66 ligands to cells expressing this collection of NgR mutant proteins was assessed at concentrations equal to the predetermined Kd's of the ligands (FIG. 2b, d).

Ala-substituted human NgR mutants were tested for their binding to AP-Aβ(1-28) and AP-Nogo-66. There are three categories: (a) NgR mutants that lose binding to both ligands, (b) mutants that maintain binding to all NgR ligands, and (c) differential binding mutants that bind AP-Nogo-66, but not AP-Aβ(1-28). A large group of amino acids are unnecessary for the binding of either ligand (Table 3). This includes all of the residues examined from the convex side of the NgR LRR domain. Another subset of amino acids are essential for the binding of both AB(1-28) and Nogo-66 (Table 3). Since these amino acids do not alter the localization or molecular size of NgR protein, and are clustered in close proximity on the concave surface, we hypothesize that they form a core ligand binding site. This is consistent with the observation that for other LRR proteins, such as follicle-stimulating hormone receptor, ligand binding predominantly occurs on the concave side. Fan Q. R. and Hendrickson W. A., Nature 433:269-277 (2005). Without structural studies, the possibility that these mutations prevent native NgR protein folding cannot be excluded. Most interesting are a third group of amino acids, for which Ala substitution results in NgR binding of Nogo-66 but not A13(1-28) (Table 3). Since AP-Nogo-66 binding is indistinguishable from wild type NgR, aberrant protein folding is unlikely to be the basis for reduced Aβ binding. Instead, NgR amino acids 210, 256, 259 and 284 are likely to contribute selectively to Aβ but not Nogo-66 interaction.

TABLE 3
Summary of human NgR mutants: list of residues mutated to alanine

| | Binding to AP-Ng-66 | Differential |
|---|---|---|
| No Binding | and AP-Aβ28 | Binding |
| 163 | 61 | 210 |
| 82, 179 | 92 | 256, 259, 284 |
| 133, 136 | 108 | |
| 158, 160 | 122 | |
| 182, 186 | 127 | |
| 211, 213 | 131 | |
| 232, 234 | 138 | |
| 111, 113, 114 | 139 | |
| 182, 186, 210 | 151 | |
| 111, 113, 114, 138 | 176 | |
| 182, 186, 158, 160 | 179 | |
| 189, 191, 211, 213 | 227 | |
| 211, 213, 237, 256, 259, 284 | 237 | |
| 171, 172, 175, 176, 196, 199, | 250 | |
| 220, 223, 224, 250 | | |
| 67, 68, 71 | 259 | |
| 67, 68, 71, 89, 90, 92 | 108, 131 | |
| 87, 89, 133, 136 | 114, 117 | |
| Negative control | 127, 151 | |
| | 127, 176 | |
| | 143, 144 | |
| | 189, 191 | |
| | 196, 199 | |
| | 202, 205 | |
| | 256, 259 | |
| | 267, 269 | |
| | 277, 279 | |
| | 114, 117, 139 | |
| | 189, 191, 237 | |
| | 189, 191, 284 | |
| | 202, 205, 227 | |
| | 202, 205, 250 | |
| | 220, 223, 224 | |
| | 237, 256, 259 | |
| | 296, 297, 300 | |
| | 171, 172, 175, 176 | |
| | 292, 296, 297, 300 | |
| | 196, 199, 220, 223, 224 | |
| | 171, 172, 175, 176, | |
| | 196, 199 | |
| | 196, 199, 220, 223, | |
| | 224, 250 | |
| | 108, 131, 61 | |
| | 36, 38 | |
| | 36, 38, 61 | |
| | 61, 131, 36, 38 | |
| | 63, 65 | |
| | 78, 81 | |
| | 87, 89 | |
| | 89, 90, 114, 117 | |
| | 95, 97 | |
| | 95, 97, 117, 119, 120, | |
| | 188, 189 | |
| | 95, 97, 122 | |
| | Wild type | |

### Example 3

### NgR(310)ecto-Fc Treatment Acts Peripherally to Alter the Plasma/Brain Aβ Ratio

While endogenous NgR plays a role in limiting Aβ production and deposition, the affinity of NgR for the central domain of Aβ suggests that it might promote peripheral clearance if delivered outside of the CNS. To examine whether rat NgR(310)ecto-Fc administered subcutaneously enters the brain of mouse, the presence of NgR(310)ecto-Fc in brain lysates was assayed. The NgR(310)ecto-Fc fusion protein or control rat IG was concentrated by protein A/G affinity chromatography. To administer rat NgR(310)ecto-Fc protein, APPswe/presenilin-1 (Psen-1)ΔE9 mice (Park et al., J Neurosci 26:1386-1395 (2006)) from Jackson Laboratories (Bar Harbor, Me.) (Stock #04462) were anesthetized with isoflurane and oxygen and an ALZET osmotic pump 2004 was subcutaneously inserted over the scapula and allowed to rest between fascia. The pump delivered 0.25 μl/hr for 28 days of a 1.2 μg/μl solution of rat NgR(310)ecto-Fc or rat IgG in PBS. Pumps were replaced after 28 days for total treatment duration of 12 weeks. The anti-Aβ (6E10) antibody was from Chemicon. DAB staining reagents were from Vector. The dose of each protein was 0.27 mg/kg/d.

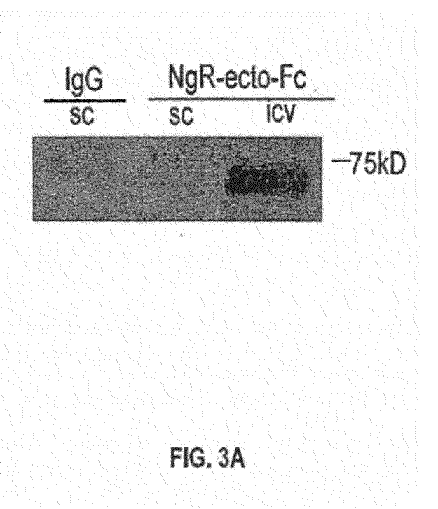

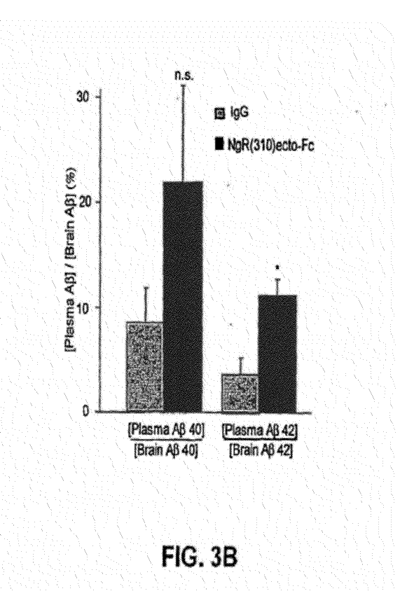

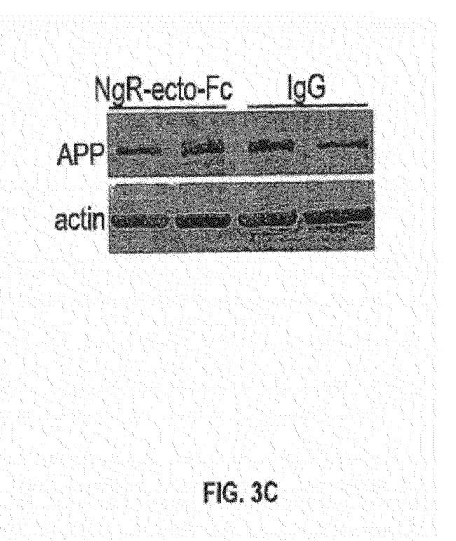

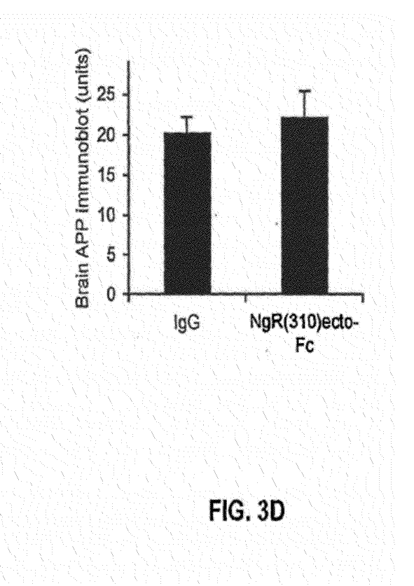

Brains from subcutaneously treated APPswe/Psen-1ΔE9 transgenic mice were homogenized in PBS plus Protease Inhibitor Cocktail (Roche). The particulate fractions were collected by centrifugation at 100,000×g for 20 min Membranes were resuspended in PBS (1 gm brain wet weight/ml) and solubilized in 1% Triton X-100. The detergent extract was subjected to Protein AIG Plus Sepharose (Pierce, Rockford, Ill.) immunoprecipitation and analyzed by anti-NgR polyclonal antibody from R&D Systems, Inc. (AF1440). While intracerebroventricular administration leads to easily detected NgR(310)ecto-Fc levels in brain tissue, no NgR(310)ecto-Fc is detected centrally after subcutaneous treatment (FIG. 3a). This is consistent with the hypothesis that NgR(310)ecto-Fc cannot pass the BBB to an appreciable degree in APPswe/Psen-16E9 mice.

To the extent that NgR(310)ecto-Fc functions as a peripheral sink for Aβ, the ratio of plasma to brain Aβ should be elevated, as shown for anti-Aβ treatment. Levels of Aβ□40 and Aβ□42 were assessed by enzyme-linked immunosorbent assay (ELISA) in brain and plasma samples from peripherally treated mice (FIG. 3b). After three months of subcutaneous treatment, there is a significant increase in the plasma:brain Aβ(1-42) ratio, *, p<0.05, ANOVA. Aβ ELISA assays were performed according to manufacturer's protocol (Biosource, Inc). Subcutaneous treatment with NgR(310)ecto-Fc increased plasma:brain ratios for Aβ more than two-fold. Previously, we noted that central, i.c.v.-administered NgR(310)ecto-Fc reduces levels of sAPPα and sAPPβ protein in the brain. Park et al., J Neurosci 26:1386-1395 (2006). However, brain APP levels are not altered by subcutaneous NgR(310)ecto-Fc treatment (FIG. 3c, d). Mean±sem from n=4-5 mice.

### Example 4

### Reduction of Aβ Plaque Load, Neuritic Dystrophy, and Astrocytosis in Ngr(310)ecto-Fc-treated APPswe/PSEN-14E9 mice

The restriction of subcutaneous NgR(310)ecto-Fc to the periphery allows an assessment of its effect as a “sink” on central AB burden. Treatment of APPswe/PS-1ΔE9 transgenic mice was initiated at 7 months of age when the mice have become symptomatic, as judged by Aβ deposition in brain and by reduced spatial memory function (see below). After 3 months of treatment with 0.27 mg/kg/day of subcutaneous NgR(310)ecto-Fc versus IgG (0.6 mg of total protein), the brain was examined by immunohistochemistry and ELISA. Aβ plaques in parasagittal sections were fixed by paraformaldehyde and labeled with anti-Aβ-(1-17) 6E10 antibody after 0.1 M formic acid treatment. Plaque area was quantitated using NIH Image as a percentage of total cerebral cortical area for two sections from each animal. Neuritic dystrophy and reactive astrocytosis were visualized by staining with monoclonal anti-synaptophysin GA-5 (Sigma) and monoclonal anti-GFAP SY 38 (Chemicon) in parasagittal paraffin-embedded sections. The area of cerebral cortex and hippocampus occupied by clusters of dystrophic neurites and reactive astrocytes were measured as a percentage of total area by the same method as AB plaque load. Data are ±SEM from 9 mice in rat IgG treated group and 7 mice from NgR(310)ecto-Fc treated group.

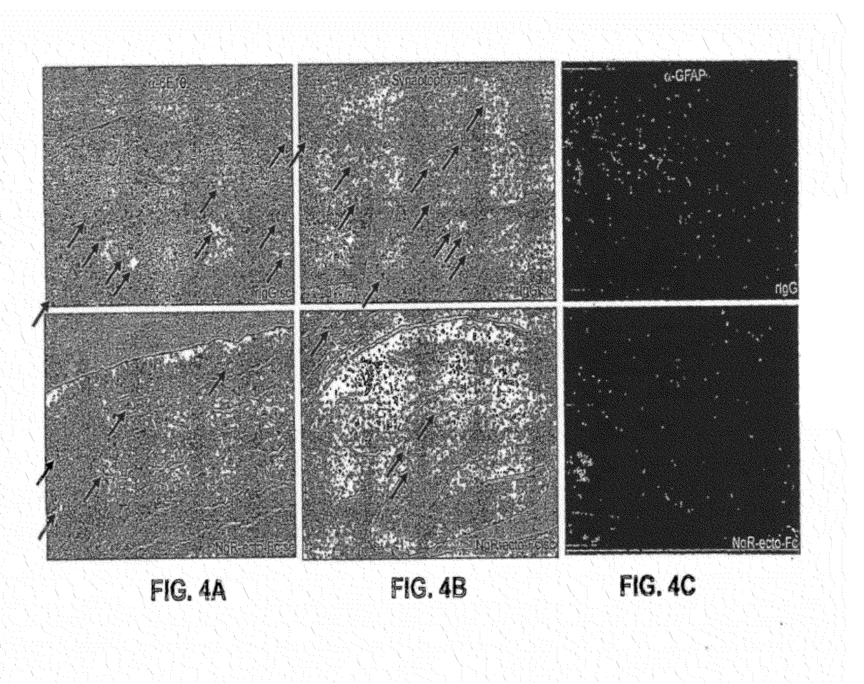

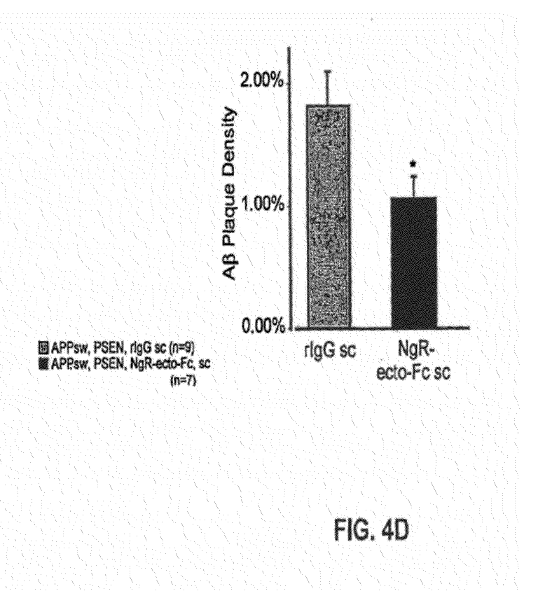

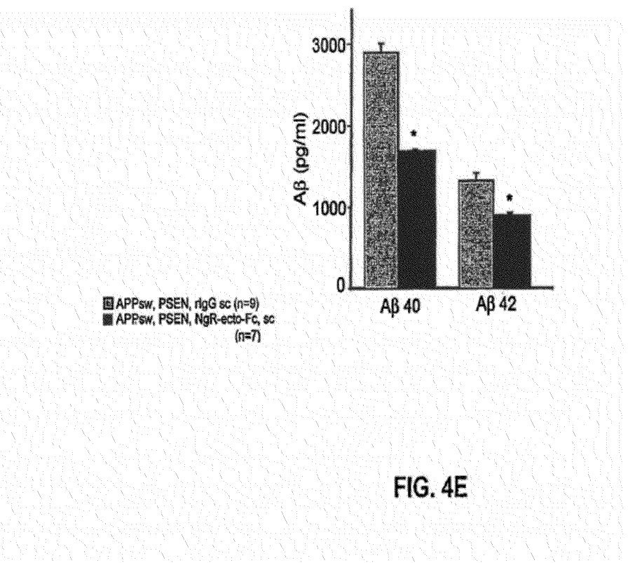

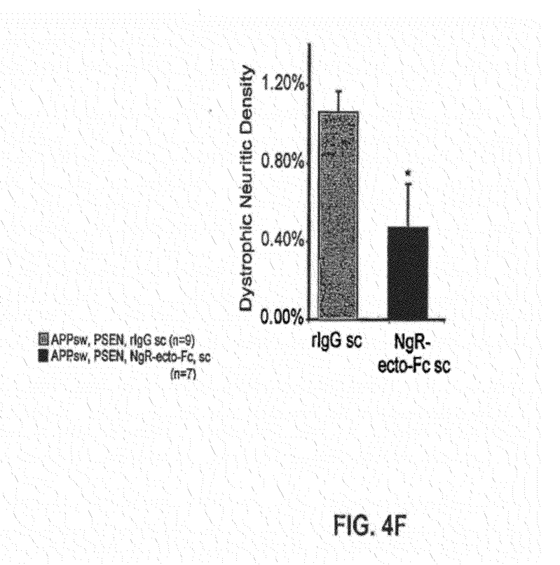

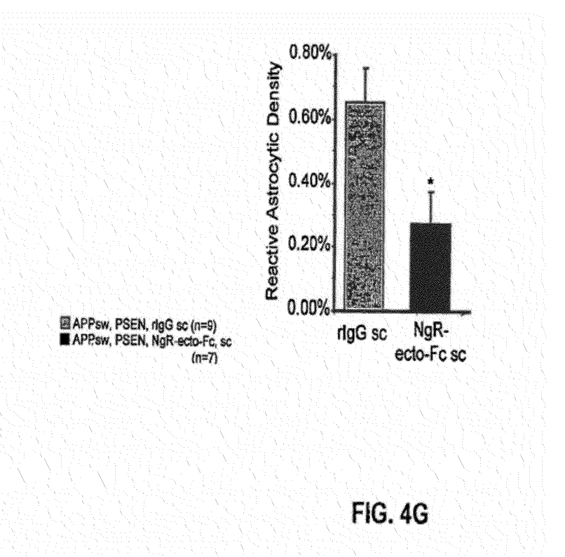

The total Aβ(1-40) and Aβ(1-42) levels as well as Aβ plaque are decreased significantly by NgR(310)ecto-Fc, to a level approximately 50% of control (FIG. 4a, d, e). In parallel, dystrophic neurites detected by anti-synaptophysin staining are decreased by peripheral NgR(310)ecto-Fc treatment (FIG. 4b, f). Astrogliosis detected by anti-GFAP staining intensity was also reduced significantly by therapy with peripheral NgR(310)ecto-Fc (FIG. 4c, g). Thus, delayed subcutaneous administration of NgR(310)ecto-Fc suppresses histologic evidence of Aβ-associated disease in transgenic mice.

### Example 5

### Subcutaneous Treatment of NgR(310)ecto-Fc Improves Radial Arm Water Maze Performance in APPswe/PSEN-14E9 Transgenic Mice

The ability of subcutaneous NgR(310)ecto-Fc therapy to reduce Aβ plaque is encouraging, but cognitive performance is the relevant symptom in clinical AD. To assess APPswe/PS-1ΔE9 transgene-related impairments in spatial memory, a modified radial arm water maze paradigm (RAWM) was employed. Morgan et al., Nature 408:982-985 (2000). A modified radial arm water maze testing protocol was based on personal communication with D. Morgan (Morgan et al., Nature 408:982-985 (2000)). The maze consisted of a circular pool one meter in diameter with six swim alleys nineteen cm wide that radiated out from a 40 cm open central area and a submerged escape platform was located at the end of one arm. Spatial cues were presented on the walls and at the end of each arm. The behaviorist was blind to treatment. To control for vision, motivation and swimming, mice were tested in an open water visual platform paradigm for up to one minute and latency times were recorded. Next, mice were placed in a random arm according to an Excel function=MOD($CELL+RANDBETWEEN(1,5),6), where $CELL is the location of the hidden platform. Each mouse was allowed to swim up to one minute to find the escape platform. Upon entering an incorrect arm (all four paws within that swim alley) or failing to select an arm after twenty seconds, the mouse was pulled back to the start arm and charged an error. All mice spent 30 seconds on the platform following each trial before beginning the next trial. Thereafter, the mouse was tested four more times, constituting a learning block. Mice were allowed to rest for 30 minutes between learning blocks. In total, mice were tested over three learning blocks over the first day and on the following day another three learning blocks were repeated.

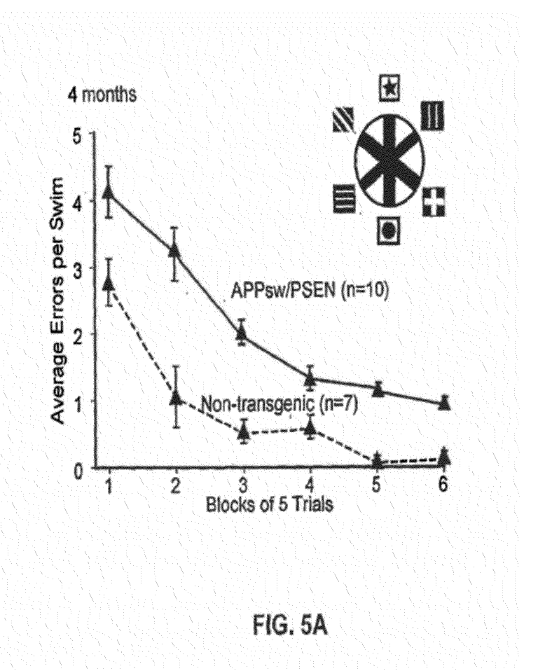

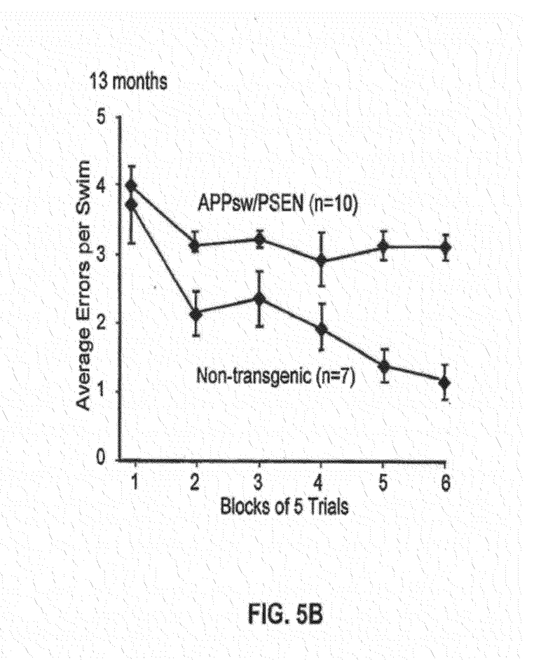

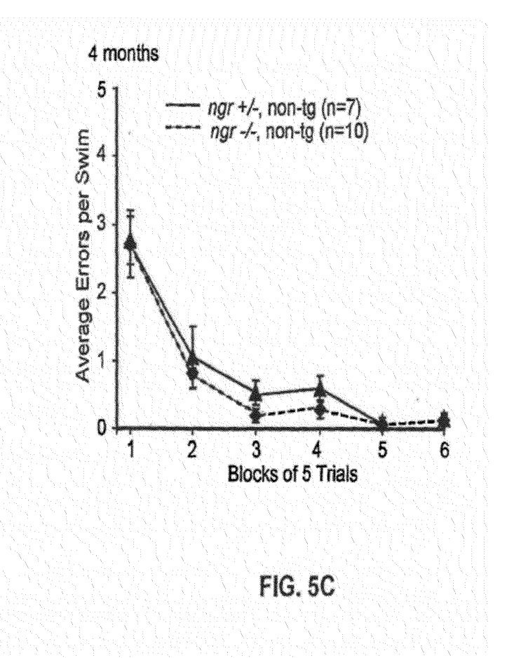

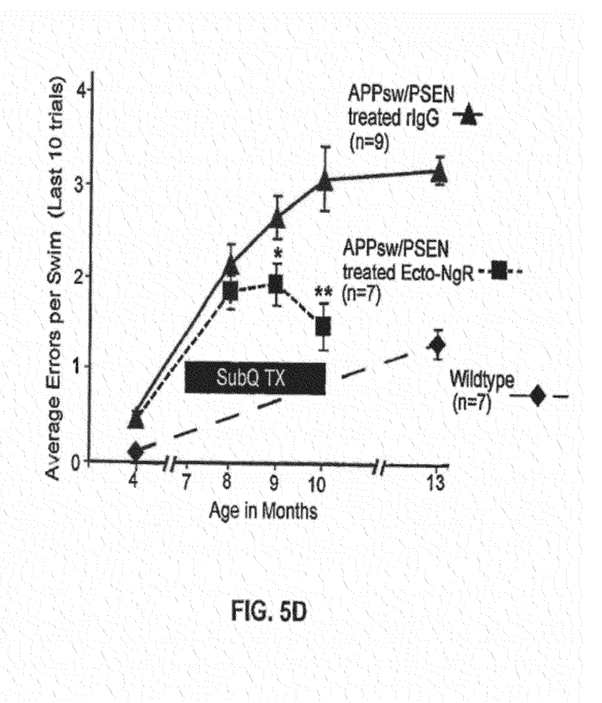

Short-term spatial memory deficits are apparent in APPswe/PS-1ΔE9 versus wild type littermate mice by 4 months (FIG. 5a). By 13 months of age, wild type mice perform less well at this task than do young mice, while APPswe/PS-1ΔE9 transgenic mice are completely unable to learn the task in our training paradigm illustrating disease progression (FIG. 5b). As a control, loss of NgR expression (in ngr −/− mice) does not significantly alter RAWM performance (FIG. 5c).

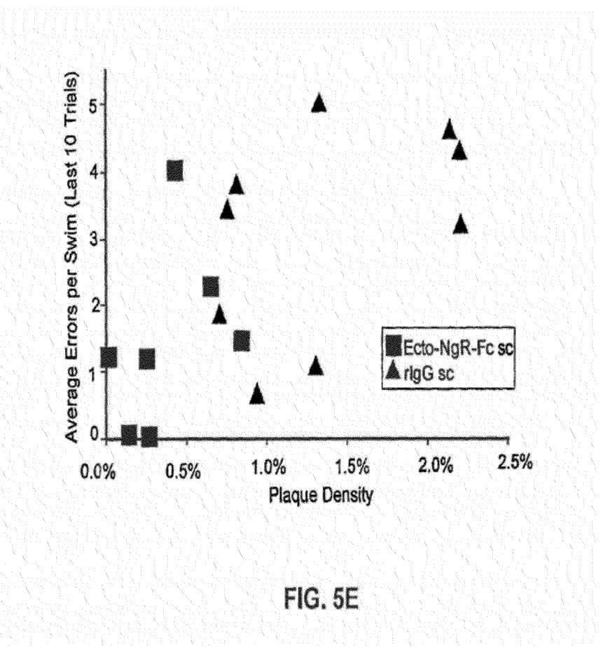

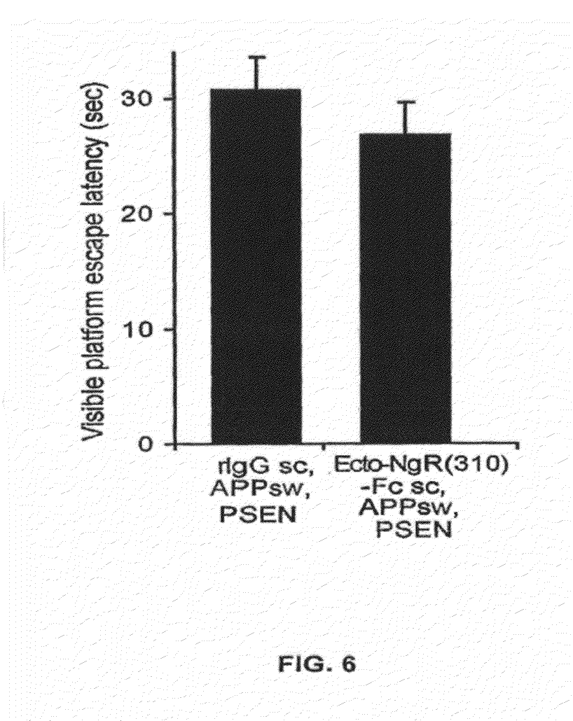

The number of swim errors made by APPswe/PS-1ΔE9 mice after 25-29 training trials increases steadily at 8, 9 and 10 months when mice receive control IgG therapy subcutaneously for 1, 2 or 3 months. In contrast, mice treated with subcutaneous NgR(310)ecto-Fc exhibit a halt in disease progression, and show a trend towards improved performance after 3 months, by 10 months of age (FIG. 5d). RAWM errors are significantly reduced after two months and after three months of subcutaneous NgR(310)ecto-Fc treatment compared to rIgG-treated mice (ANOVA, P<0.05 and 0.02, respectively). These differences are related to improved memory function rather than altered vision, motivation or motor capacity, since no significant difference was observed in visible platform escape latencies between these groups (FIG. 6). Mean±sem from n=7-9 mice per group. There is a positive correlation between the average RAWM errors and the density of Aβ-immunoreactive deposits across the two groups (FIG. 5e).

As those skilled in the art will appreciate, numerous changes and modifications may be made to the preferred embodiments of the invention without departing from the spirit of the invention. It is intended that all such variations fall within the scope of the invention.

1. A method of increasing the plasma to brain ratio of Aβ peptide in a mammal or enhancing Aβ clearance from the brain of a mammal, comprising administering to a mammal in need thereof a therapeutically effective amount of a soluble Nogo receptor polypeptide, wherein said administration is peripheral to the central nervous system.

2. (canceled)

3. (canceled)

4. A method of reducing the number of Aβ plaques or the size of Aβ plaques in the brain of a mammal, comprising administering to a mammal in need thereof a therapeutically effective amount of a soluble Nogo receptor polypeptide, wherein said administration is peripheral to the central nervous system.

5. (canceled)

6. A method of treating a disease associated with Aβ plaque accumulation in a mammal comprising administering to a mammal in need thereof a therapeutically effective amount of a soluble Nogo receptor polypeptide, wherein said administration is peripheral to the central nervous system.

7. The method of claim 6, wherein said disease is selected from the group consisting of Alzheimer's disease, mild cognitive impairment, mild-to-moderate cognitive impairment, vascular dementia, cerebral amyloid angiopathy, hereditary cerebral hemorrhage, senile dementia, Down's syndrome, inclusion body myositis, age-related macular degeneration, primary amyloidosis, secondary amyloidosis and a condition associated with Alzheimer's disease.

8. The method of claim 7, wherein said condition associated with Alzheimer's disease is selected from the group consisting of hypothyroidism, cerebrovascular disease, cardiovascular disease, memory loss, anxiety, a behavioral dysfunction, a neurological condition, and a psychological condition.

9. (canceled)

10. (canceled)

11. (canceled)

12. The method of claim 1, wherein said mammal is a human.

13. The method of claim 1, wherein said soluble Nogo receptor polypeptide is administered subcutaneously, parenterally, intravenously, intramuscularly, intraperitoneally, transdermally, inhalationaly or buccally.

14. The method of claim 1, wherein said soluble NgR1 polypeptide is 90% identical to a reference amino acid sequence is selected from the group consisting of:
(i) amino acids 27 to 310 of SEQ ID NO:2;
(ii) amino acids 27 to 344 of SEQ ID NO:2;
(iii) amino acids 27 to 445 of SEQ ID NO:2;
(iv) amino acids 27 to 309 of SEQ ID NO:2;
(v) amino acids 1 to 310 of SEQ ID NO:2;
(vi) amino acids 1 to 344 of SEQ ID NO:2;
(vii) amino acids 1 to 445 of SEQ ID NO:2;
(viii) amino acids 1 to 309 of SEQ ID NO:2;
(ix) variants or derivatives of any of said reference amino acid sequences, and
(x) a combination of one or more of said reference amino acid sequences or variants or derivatives thereof.

15. The method of claim 14, wherein said soluble NgR1 polypeptide is selected from the group consisting of:
(i) amino acids 27 to 310 of SEQ ID NO:2;
(ii) amino acids 27 to 344 of SEQ ID NO:2;
(iii) amino acids 27 to 445 of SEQ ID NO:2;
(iv) amino acids 27 to 309 of SEQ ID NO:2;
(v) amino acids 1 to 310 of SEQ ID NO:2;
(vi) amino acids 1 to 344 of SEQ ID NO:2;
(vii) amino acids 1 to 445 of SEQ ID NO:2;
(viii) amino acids 1 to 309 of SEQ ID NO:2;
(ix) variants or derivatives of any of said polypeptides; and
(x) a combination of one or more of said polypeptides or variants or derivatives thereof.

16. (canceled)

17. (canceled)

18. (canceled)

19. (canceled)

20. (canceled)

21. (canceled)

22. (canceled)

23. (canceled)

24. The method of claim 1, wherein said soluble Nogo receptor polypeptide comprises a first polypeptide fragment and a second polypeptide fragment, wherein said first polypeptide fragment comprises an amino acid sequence identical to a first reference amino acid sequence, except for up to twenty individual amino acid substitutions, wherein said first reference amino acid sequence is selected from the group consisting of:
(a) amino acids a to 445 of SEQ ID NO:2,
(b) amino acids 27 to b of SEQ ID NO:2, and
(c) amino acids a to b of SEQ ID NO:2,
wherein a is any integer from 25 to 35, and b is any integer from 300 to 450; and
wherein said second polypeptide fragment comprises an amino acid sequence identical to a second reference amino acid sequence, except for up to twenty individual amino acid substitutions, wherein said second reference amino acid sequence is selected from the group consisting of:
(a) amino acids c to 445 of SEQ ID NO:2,
(b) amino acids 27 to d of SEQ ID NO:2, and
(c) amino acids c to d of SEQ ID NO:2,
wherein c is any integer from 25 to 35, and d is any integer from 300 to 450.

25. (canceled)

26. (canceled)

27. (canceled)

28. The method of claim 14, wherein at least one amino acid residue of said, soluble NgR1 polypeptide is substituted with a different amino acid.

29. (canceled)

30. (canceled)

31. The method of claim 14, wherein said soluble NgR1 polypeptide is a cyclic polypeptide.

32. (canceled)

33. (canceled)

34. (canceled)

35. (canceled)

36. (canceled)

37. The method of claim 14, wherein said soluble NgR1 polypeptide further comprises a non-NgR1 moiety.

38. The method of claim 37, wherein said non-NgR1 moiety is a heterologous polypeptide fused to said soluble NgR1 polypeptide.

39. (canceled)

40. (canceled)

41. (canceled)

42. (canceled)

43. (canceled)

44. (canceled)

45. The method of claim 37, wherein said soluble NgR1 polypeptide is conjugated to a polymer.

46. (canceled)

47. (canceled)

48. (canceled)

49. (canceled)

50. (canceled)

51. The method of claim 1, wherein the therapeutically effective amount is from 0.001 mg/kg to 10 mg/kg of soluble Nogo receptor polypeptide.

52. (canceled)

53. (canceled)

54. The method of claim 1, wherein the soluble Nogo receptor polypeptide does not cross the blood-brain barrier.

55. The method of claim 1, wherein said soluble Nogo receptor polypeptide is coadministered with one or more anti-Aβ antibodies.

56. The method of claim 55, wherein said soluble Nogo receptor polypeptide is coadministered with one or more additional therapeutic agents, selected from the group consisting of an adrenergic agent, anti-adrenergic agent, anti-androgen agent, anti-anginal agent, anti-anxiety agent, anticonvulsant agent, antidepressant agent, anti-epileptic agent, antihyperlipidemic agent, antihyperlipoproteinemic agent, antihypertensive agent, anti-inflammatory agent, antiobessional agent, antiparkinsonian agent, antipsychotic agent, adrenocortical steroid; adrenocortical suppressant; aldosterone antagonist; amino acid; anabolic steroid; analeptic agent; androgen; blood glucose regulator; cardioprotectant agent; cardiovascular agent; cholinergic agonist or antagonist; cholinesterase deactivator or inhibitor, cognition adjuvant or enhancer; dopaminergic agent; enzyme inhibitor, estrogen, free oxygen radical scavenger; GABA agonist; glutamate antagonist; hormone; hypocholesterolemic agent; hypolipidemic agent; hypotensive agent; immunizing agent; immunostimulant agent; monoamine oxidase inhibitor, neuroprotective agent; NMDA antagonist; AMPA antagonist, competitive or-non-competitive NMDA antagonist; opioid antagonist; potassium channel opener; non-hormonal sterol derivative; post-stroke and post-head trauma treatment; prostaglandin agent; psychotropic agent; relaxant agent; sedative agent; sedative-hypnotic agent; selective adenosine antagonist; serotonin antagonist; serotonin inhibitor; selective serotonin uptake inhibitor; serotonin receptor antagonist; sodium and calcium channel blocker; steroid; stimulant; and thyroid hormone and inhibitor agents.


SEQUENCE LISTING (33 sequences)

SEQ ID NO: 1 - DNA, 1719 bp, Homo sapiens
agcccagccagagccgggcggagcggagcgcgccgagcctcgtcccgcggccgggccggggccgggccgtagcggcggcgcctggatgcggacccggccgcggggagacgggcgcccgccccgaaacgactttcagtccccgacgcgccccgcccaacccctacgatgaagagggcgtccgctggagggagccggctgctggcatgggtgctgtggctgcaggcctggcaggtggcagccccatgcccaggtgcctgcgtatgctacaatgagcccaaggtgacgacaagctgcccccagcagggcctgcaggctgtgcccgtgggcatccctgctgccagccagcgcatcttcctgcacggcaaccgcatctcgcatgtgccagctgccagcttccgtgcctgccgcaacctcaccatcctgtggctgcactcgaatgtgctggcccgaattgatgcggctgccttcactggcctggccctcctggagcagctggacctcagcgataatgcacagctccggtctgtggaccctgccacattccacggcctgggccgcctacacacgctgcacctggaccgctgcggcctgcaggagctgggcccggggctgttccgcggcctggctgccctgcagtacctctacctgcaggacaacgcgctgcaggcactgcctgatgacaccttccgcgacctgggcaacctcacacacctcttcctgcacggcaaccgcatctccagcgtgcccgagcgcgccttccgtgggctgcacagcctcgaccgtctcctactgcaccagaaccgcgtggcccatgtgcacccgcatgccttccgtgaccttggccgcctcatgacactctatctgtttgccaacaatctatcagcgctgcccactgaggccctggcccccctgcgtgccctgcagtacctgaggctcaacgacaacccctgggtgtgtgactgccgggcacgcccactctgggcctggctgcagaagttccgcggctcctcctccgaggtgccctgcagcctcccgcaacgcctggctggccgtgacctcaaacgcctagctgccaatgacctgcagggctgcgctgtggccaccggcccttaccatcccatctggaccggcagggccaccgatgaggagccgctggggcttcccaagtgctgccagccagatgccgctgacaaggcctcagtactggagcctggaagaccagcttcggcaggcaatgcgctgaagggacgcgtgccgcccggtgacagcccgccgggcaacggctctggcccacggcacatcaatgactcaccctttgggactctgcctggctctgctgagcccccgctcactgcagtgcggcccgagggctccgagccaccagggttccccacctcgggccctcgccggaggccaggctgttcacgcaagaaccgcacccgcagccactgccgtctgggccaggcaggcagcgggggtggcgggactggtgactcagaaggctcaggtgccctacccagcctcacctgcagcctcacccccctgggcctggcgctggtgctgtggacagtgcttgggccctgctgacccccagcggacacaagagcgtgctcagcagccaggtgtgtgtacatacggggtctctctccacgccgccaagccagccgggcggccgacccgtggggcaggccaggccaggtcctccctgatggacgcctg

SEQ ID NO: 2 - protein, 473 aa, Homo sapiens
MKRASAGGSRLLAWVLWLQAWQVAAPCPGACVCYNEPKVTTSCPQQGLQAVPVGIPAASQRIFLHGNRISHVPAASFRACRNLTILWLHSNVLARIDAAAFTGLALLEQLDLSDNAQLRSVDPATFHGLGRLHTLHLDRCGLQELGPGLFRGLAALQYLYLQDNALQALPDDTFRDLGNLTHLFLHGNRISSVPERAFRGLHSLDRLLLHQNRVAHVHPHAFRDLGRLMTLYLFANNLSALPTEALAPLRALQYLRLNDNPWVCDCRARPLWAWLQKFRGSSSEVPCSLPQRLAGRDLKRLAANDLQGCAVATGPYHPIWTGRATDEEPLGLPKCCQPDAADKASVLEPGRPASAGNALKGRVPPGDSPPGNGSGPRHINDSPFGTLPGSAEPPLTAVRPEGSEPPGFPTSGPRRRPGCSRKNRTRSHCRLGQAGSGGGGTGDSEGSGALPSLTCSLTPLGLALVLWTVLGPC

SEQ ID NO: 3 - DNA, 1422 bp, Rattus
atgaagagggcgtcctccggaggaagccggctgccgacatgggtgttatggctacaggcctggagggtagcaacgccctgccctggtgcctgtgtgtgctacaatgagcccaaggtcacaacaagccgcccccagcagggcctgcaggctgtacccgctggcatcccagcctccagccagagaatcttcctgcacggcaaccgaatctcttacgtgccagccgccagcttccagtcatgccggaatctcaccatcctgtggctgcactcaaatgcgctggccgggattgatgccgcggccttcactggtctgaccctcctggagcaactagatcttagtgacaatgcacagctccgtgtcgtggaccccaccacgttccgtggcctgggccacctgcacacgctgcacctagaccgatgcggcctgcaggagctggggcctggcctattccgtgggctggcagctctgcagtacctctacctacaagacaacaacctgcaggcacttcccgacaacaccttccgagacctgggcaacctcacgcatctctttctgcatggcaaccgtatccccagtgttcctgagcacgctttccgtggcttgcacagtcttgaccgtctcctcttgcaccagaaccatgtggctcgtgtgcacccacatgccttccgggaccttggccgactcatgaccctctacctgtttgccaacaacctctccatgctccccgcagaggtcctagtgcccctgaggtctctgcagtacctgcgactcaatgacaacccctgggtgtgtgactgcagggcacgtccgctctgggcctggctgcagaagttccgaggttcctcatccggggtgcccagcaacctaccccaacgcctggcaggccgtgatctgaagcgcctggctaccagtgacttagagggttgtgctgtggcttcggggcccttccgtcccttccagaccaatcagctcactgatgaggagctgctgggcctccccaagtgctgccagccggatgctgcagacaaggcctcagtactggaacccgggaggccggcgtctgttggaaatgcactcaagggacgtgtgcctcccggtgacactccaccaggcaatggctcaggcccacggcacatcaatgactctccatttgggactttgcccggctctgcagagcccccactgactgccctgcggcctgggggttccgagcccccgggactgcccaccacgggcccccgcaggaggccaggttgttccagaaagaaccgcacccgtagccactgccgtctgggccaggcaggaagtgggagcagtggaactggggatgcagaaggttcgggggccctgcctgccctggcctgcagccttgctcctctgggccttgcactggtactttggacagtgcttgggccctgctga

SEQ ID NO: 4 - protein, 473 aa, Rattus
MKRASSGGSRLLAWVLWLQAWRVATPCPGACVCYNEPKVTTSCPQQGLQAVPTGIPASSQRIFLHGNRISHVPAASFQSCRNLTILWLHSNALARIDAAAFTGLTLLEQLDLSDNAQLHVVDPTTFHGLGHLHTLHLDRCGLRELGPGLFRGLAALQYLYLQDNNLQALPDNTFRDLGNLTHLFLHGNRIPSVPEHAFRGLHSLDRLLLHQNHVARVHPHAFRDLGRLMTLYLFANNLSMLPAEVLMPLRSLQYLRLNDNPWVCDCRARPLWAWLQKFRGSSSEVPCNLPQRLADRDLKRLAASDLEGCAVASGPFRPIQTSQLTDEELLSLPKCCQPDAADKASVLEPGRPASAGNALKGRVPPGDTPPGNGSGPRHINDSPFGTLPSSAEPPLTALRPGGSEPPGLPTTGPRRRPGCSRKNRTRSHCRLGQAGSGASGTGDAEGSGALPALACSLAPLGLALVLWTVLGPC

SEQ ID NO: 5 - DNA, 1892 bp, Mus musculus
agccgcagcccgcgagcccagcccggcccggtagagcggagcgccggagcctcgtcccgcggccgggccgggaccgggccggagcagcggcgcctggatgcggacccggccgcgcgcagacgggcgcccgccccgaagccgcttccagtgcccgacgcgccccgctcgaccccgaagatgaagagggcgtcctccggaggaagcaggctgctggcatgggtgttatggctacaggcctggagggtagcaacaccatgccctggtgcttgtgtgtgctacaatgagcccaaggtaacaacaagctgcccccagcagggtctgcaggctgtgcccactggcatcccagcctctagccagcgaatcttcctgcatggcaaccgaatctctcacgtgccagctgcgagcttccagtcatgccgaaatctcactatcctgtggctgcactctaatgcgctggctcggatcgatgctgctgccttcactggtctgaccctcctggagcaactagatcttagtgataatgcacagcttcatgtcgtggaccctaccacgttccacggcctgggccacctgcacacactgcacctagaccgatgtggcctgcgggagctgggtcccggcctattccgtggactagcagctctgcagtacctctacctacaagacaacaatctgcaggcactccctgacaacacctttcgagacctgggcaacctcacgcatctctttctgcatggcaaccgtatccccagtgtgcctgagcacgctttccgtggcctgcacagtcttgaccgcctcctcttgcaccagaaccatgtggctcgtgtgcacccacatgccttccgggaccttggccgcctcatgaccctctacctgtttgccaacaacctctccatgctgcctgcagaggtcctaatgcccctgaggtctctgcagtacctgcgactcaatgacaacccctgggtgtgtgactgccgggcacgtccactctgggcctggctgcagaagttccgaggttcctcatcagaggtgccctgcaacctgccccaacgcctggcagaccgtgatcttaagcgcctcgctgccagtgacctagagggctgtgctgtggcttcaggacccttccgtcccatccagaccagtcagctcactgatgaggagctgctgagcctccccaagtgctgccagccagatgctgcagacaaagcctcagtactggaacccgggaggccagcttctgccggaaacgccctcaagggacgtgtgcctcccggtgacactccaccaggcaatggctcaggccctcggcacatcaatgactctccatttggaactttgcccagctctgcagagcccccactgactgccctgcggcctgggggttccgagccaccaggacttcccaccactggtccccgcaggaggccaggttgttcccggaagaatcgcacccgcagccactgccgtctgggccaggcgggaagtggggccagtggaacaggggacgcagagggttcaggggctctgcctgctctggcctgcagccttgctcctctgggccttgcactggtactttggacagtgcttgggccctgctgaccagccaccagccaccaggtgtgtgtacatatggggtctccctccacgccgccagccagagccagggacaggctctgaggggcaggccaggccctccctgacagatgcctccccaccagcccacccccatctccaccccatcatgtttacagggttccgggggtggcgtttgttccagaacgccacctcccacccggatcgcggtatatagagatatgaattttattttacttgtgtaaaatatcggatgacgtggaataaagagctcttttcttaaaaaaaaaaaaaaaaaa

SEQ ID NO: 6 - protein, 473 aa, Mus musculus; identical to SEQ ID NO: 4

SEQ ID NO: 7 - protein, 344 aa, Homo sapiens
MKRASAGGSRLLAWVLWLQAWQVAAPCPGACVCYNEPKVTTSCPQQGLQAVPVGIPAASQRIFLHGNRISHVPAASFRACRNLTILWLHSNVLARIDAAAFTGLALLEQLDLSDNAQLRSVDPATFHGLGRLHTLHLDRCGLQELGPGLFRGLAALQYLYLQDNALQALPDDTFRDLGNLTHLFLHGNRISSVPERAFRGLHSLDRLLLHQNRVAHVHPHAFRDLGRLMTLYLFANNLSALPTEALAPLRALQYLRLNDNPWVCDCRARPLWAWLQKFRGSSSEVPCSLPQRLAGRDLKRLAANDLQGCAVATGPYHPIWTGRATDEEPLGLPKCCQPDAADKA

SEQ ID NO: 8 - protein, 310 aa, Homo sapiens
MKRASAGGSRLLAWVLWLQAWQVAAPCPGACVCYNEPKVTTSCPQQGLQAVPVGIPAASQRIFLHGNRISHVPAASFRACRNLTILWLHSNVLARIDAAAFTGLALLEQLDLSDNAQLRSVDPATFHGLGRLHTLHLDRCGLQELGPGLFRGLAALQYLYLQDNALQALPDDTFRDLGNLTHLFLHGNRISSVPERAFRGLHSLDRLLLHQNRVAHVHPHAFRDLGRLMTLYLFANNLSALPTEALAPLRALQYLRLNDNPWVCDCRARPLWAWLQKFRGSSSEVPCSLPQRLAGRDLKRLAANDLQGCA

SEQ ID NO: 9 - protein, 344 aa, Rattus
MKRASSGGSRLPTWVLWLQAWRVATPCPGACVCYNEPKVTTSRPQQGLQAVPAGIPASSQRIFLHGNRISYVPAASFQSCRNLTILWLHSNALAGIDAAAFTGLTLLEQLDLSDNAQLRVVDPTTFRGLGHLHTLHLDRCGLQELGPGLFRGLAALQYLYLQDNNLQALPDNTFRDLGNLTHLFLHGNRIPSVPEHAFRGLHSLDRLLLHQNHVARVHPHAFRDLGRLMTLYLFANNLSMLPAEVLVPLRSLQYLRLNDNPWVCDCRARPLWAWLQKFRGSSSGVPSNLPQRLAGRDLKRLATSDLEGCAVASGPFRPFQTNQLTDEELLGLPKCCQPDAADKA

SEQ ID NO: 10 - protein, 310 aa, Rattus
MKRASSGGSRLPTWVLWLQAWRVATPCPGACVCYNEPKVTTSRPQQGLQAVPAGIPASSQRIFLHGNRISYVPAASFQSCRNLTILWLHSNALAGIDAAAFTGLTLLEQLDLSDNAQLRVVDPTTFRGLGHLHTLHLDRCGLQELGPGLFRGLAALQYLYLQDNNLQALPDNTFRDLGNLTHLFLHGNRIPSVPEHAFRGLHSLDRLLLHQNHVARVHPHAFRDLGRLMTLYLFANNLSMLPAEVLVPLRSLQYLRLNDNPWVCDCRARPLWAWLQKFRGSSSGVPSNLPQRLAGRDLKRLATSDLEGCA

SEQ ID NO: 11 - protein, 310 aa, Artificial sequence
  Human NgR1 with Ala substitutions at positions 266 and 309
MKRASAGGSRLLAWVLWLQAWQVAAPCPGACVCYNEPKVTTSCPQQGLQAVPVGIPAASQRIFLHGNRISHVPAASFRACRNLTILWLHSNVLARIDAAAFTGLALLEQLDLSDNAQLRSVDPATFHGLGRLHTLHLDRCGLQELGPGLFRGLAALQYLYLQDNALQALPDDTFRDLGNLTHLFLHGNRISSVPERAFRGLHSLDRLLLHQNRVAHVHPHAFRDLGRLMTLYLFANNLSALPTEALAPLRALQYLRLNDNPWVCDARARPLWAWLQKFRGSSSEVPCSLPQRLAGRDLKRLAANDLQGAA

SEQ ID NO: 12 - protein, 310 aa, Artificial sequence
  Rat NgR1 with Ala substitutions at positions 266 and 309
MKRASSGGSRLPTWVLWLQAWRVATPCPGACVCYNEPKVTTSRPQQGLQAVPAGIPASSQRIFLHGNRISYVPAASFQSCRNLTILWLHSNALAGIDAAAFTGLTLLEQLDLSDNAQLRVVDPTTFRGLGHLHTLHLDRCGLQELGPGLFRGLAALQYLYLQDNNLQALPDNTFRDLGNLTHLFLHGNRIPSVPEHAFRGLHSLDRLLLHQNHVARVHPHAFRDLGRLMTLYLFANNLSMLPAEVLVPLRSLQYLRLNDNPWVCDARARPLWAWLQKFRGSSSGVPSNLPQRLAGRDLKRLATSDLEGAA

SEQ ID NO: 13 - protein, 344 aa, Artificial sequence
  Human NgR1 with Ala substitutions at positions 266 and 309
MKRASAGGSRLLAWVLWLQAWQVAAPCPGACVCYNEPKVTTSCPQQGLQAVPVGIPAASQRIFLHGNRISHVPAASFRACRNLTILWLHSNVLARIDAAAFTGLALLEQLDLSDNAQLRSVDPATFHGLGRLHTLHLDRCGLQELGPGLFRGLAALQYLYLQDNALQALPDDTFRDLGNLTHLFLHGNRISSVPERAFRGLHSLDRLLLHQNRVAHVHPHAFRDLGRLMTLYLFANNLSALPTEALAPLRALQYLRLNDNPWVCDARARPLWAWLQKFRGSSSEVPCSLPQRLAGRDLKRLAANDLQGAAVATGPYHPIWTGRATDEEPLGLPKCCQPDAADKA

SEQ ID NO: 14 - protein, 420 aa, Homo sapiens
MLPGLRRLLQGPASACLLLTLLALPSVTPSCPMLCTCYSSPPTVSCQANNFSSVPLSLPPSTQRLFLQNNLIRSLRPGTFGPNLLTLWLFSNNLSTIHPGTFRHLQALEELDLGDNRHLRSLEPDTFQGLERLQSLHLYRCQLSSLPGNIFRGLVSLQYLYLQENSLLHLQDDLFADLANLSHLFLHGNRLRLLTEHVFRGLGSLDRLLLHGNRLQGVHRAAFHGLSRLTILYLFNNSLASLPGEALADLPALEFLRLNANPWACDCRARPLWAWFQRARVSSSDVTCATPPERQGRDLRALRDSDFQACPPPTPTRPGSRARGNSSSNHLYGVAEAGAPPADPSTLYRDLPAEDSRGRQGGDAPTEDDYWGGYGGEDQRGEQTCPGAACQAPADSRGPALSAGLRTPLLCLLPLALHHL

SEQ ID NO: 15 - protein, 420 aa, Mus musculus; identical to SEQ ID NO: 14

SEQ ID NO: 16 - protein, 441 aa, Homo sapiens
MLRKGCCVELLLLLVAAELPLGGGCPRDCVCYPAPMTVSCQAHNFAAIPEGIPVDSERVFLQNNRIGLLQPGHFSPAMVTLWIYSNNITYIHPSTFEGFVHLEELDLGDNRQLRTLAPETFQGLVKLHALYLYKCGLSALPAGVFGGLHSLQYLYLQDNHIEYLQDDIFVDLVNLSHLFLHGNKLWSLGPGTFRGLVNLDRLLLHENQLQWVHHKAFHDLRRLTTLFLFNNSLSELQGECLAPLGALEFLRLNGNPWDCGCRARSLWEWLQRFRGSSSAVPCVSPGLRHGQDLKLLRAEDFRNCTGPASPHQIKSHTLTTTDRAARKEHHSPHGPTRSKGHPHGPRPGHRKPGKNCTNPRNRNQISKAGAGKQAPELPDYAPDYQHKFSFDIMPTARPKRKGKCARRTPIRAPSGVQQASSASSLGASLLAWTLGLAVTLR

SEQ ID NO: 17 - protein, 445 aa, Mus musculus
MLRKGCCVELLLLLLAGELPLGGGCPRDCVCYPAPMTVSCQAHNFAAIPEGIPEDSERIFLQNNRITFLQQGHFSPAMVTLWIYSNNITFIAPNTFEGFVHLEELDLGDNRQLRTLAPETFQGLVKLHALYLYKCGLSALPAGIFGGLHSLQYLYLQDNHIEYLQDDIFVDLVNLSHLFLHGNKLWSLGQGIFRGLVNLDRLLLHENQLQWVHHKAFHDLHRLTTLFLFNNSLTELQGDCLAPLVALEFLRLNGNAWDCGCRARSLWEWLRRFRGSSSAVPCATPELRQGQDLKLLRVEDFRNCTGPVSPHQIKSHTLTTSDRAARKEHHPSHGASRDKGHPHGHPPGSRSGYKKAGKNCTSHRNRNQISKVSSGKELTELQDYAPDYQHKFSFDIMPTARPKRKGKCARRTPIRAPSGVQQASSGTALGAPLLAWILGLAVTLR

SEQ ID NO: 18 - protein, 15 aa, Artificial sequence
  Linker sequence
GGGGSGGGGSGGGGS

SEQ ID NO: 19 - protein, 10 aa, Artificial sequence
  Linker sequence
GGGGSGGGGS

SEQ ID NO: 20 - protein, 15 aa, Artificial sequence
  Linker sequence
ESGRSGGGGSGGGGS

SEQ ID NO: 21 - protein, 14 aa, Artificial sequence
  Linker sequence
EGKSSGSGSESKST

SEQ ID NO: 22 - protein, 15 aa, Artificial sequence
  Linker sequence
EGKSSGSGSESKSTQ

SEQ ID NO: 23 - protein, 14 aa, Artificial sequence
  Linker sequence
EGKSSGSGSESKVD

SEQ ID NO: 24 - protein, 14 aa, Artificial sequence
  Linker sequence
GSTSGSGKSSEGKG

SEQ ID NO: 25 - protein, 18 aa, Artificial sequence
  Linker sequence
KESGSVSSEQLAQFRSLD

SEQ ID NO: 26 - protein, 16 aa, Artificial sequence
  Linker sequence
ESGSVSSEELAFRSLD

SEQ ID NO: 27 - protein, 8 aa, Artificial sequence
  Artificial Epitope
DYKDDDDK

SEQ ID NO: 28 - protein, 8 aa, Artificial sequence
  Artificial Epitope
DYKDEDDK

SEQ ID NO: 29 - protein, 9 aa, Artificial sequence
  Artificial Epitope
AWRHPQFGG

SEQ ID NO: 30 - protein, 11 aa, Artificial sequence
  Artificial Epitope
YTDIEMNRLGK

SEQ ID NO: 31 - protein, 6 aa, Artificial sequence
  Artificial Epitope
HHHHHH

SEQ ID NO: 32 - protein, 13 aa, Influenza virus
YPYDVPDYAIEGR

SEQ ID NO: 33 - protein, 11 aa, Homo sapiens
EQKLLSEEDLN